SEQUENCE LISTING (158 sequences)

SEQ ID NO: 1 - protein, 203 aa, synthetic construct
MEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 2 - protein, 73 aa, synthetic construct
MKMEELFKKHKIVAVLRANSVEEAIEKAVAVFAGGVHLIEITFTVPDADTVIKALSVLKEKGAIIGAGTVTSV

SEQ ID NO: 3 - protein, 106 aa, synthetic construct
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVF

SEQ ID NO: 4 - protein, 127 aa, synthetic construct
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDI

SEQ ID NO: 5 - protein, 128 aa, synthetic construct
EQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGKALVKGKPDEVREKAKKFVKKIR

SEQ ID NO: 6 - protein, 95 aa, synthetic construct
YMPGVMTPTELVKAMKLGHDILKLFPGEVVGPQFVKAMKGPFPNVKFVPTGGVNLDNVCKWFKAGVLAVGVGKALVKGKPDEVREKAKKFVKKIR

SEQ ID NO: 7 - protein, 74 aa, synthetic construct
LKLFPGEVVGPQFVKAMKGPFPNVKFVPTGGVNLDNVCKWFKAGVLAVGVGKALVKGKPDEVREKAKKFVKKIR

SEQ ID NO: 8 - protein, 17 aa, synthetic construct
KKVGEELIKLVTRPEDR

SEQ ID NO: 9 - protein, 18 aa, synthetic construct
KEVGKKLLLLVTDPADKK

SEQ ID NO: 10 - protein, 19 aa, synthetic construct
DKESEKLIKLETDPAMLRM

SEQ ID NO: 11 - protein, 20 aa, synthetic construct
EKEGKKALKLETDPAMKKMV

SEQ ID NO: 12 - protein, 22 aa, synthetic construct
VQAVHKKALAVAPKLTEAQALM

SEQ ID NO: 13 - protein, 18 aa, synthetic construct
ENKKVGEELIKLVTRPED

SEQ ID NO: 14 - protein, 18 aa, synthetic construct
VNNEVGKKLLLLVTDPAD

SEQ ID NO: 15 - protein, 18 aa, synthetic construct
ENEKEGKKALKLETDPAM

SEQ ID NO: 16 - protein, 18 aa, synthetic construct
EAKKESEKLIKLETDPAM

SEQ ID NO: 17 - protein, 21 aa, synthetic construct
PEVEAVHEKALAVAPKLTEAQ

SEQ ID NO: 18 - protein, 18 aa, synthetic construct
ENKKVGEELIKLVTRPED

SEQ ID NO: 19 - protein, 18 aa, synthetic construct
ENEKEGKKALKLETDPAM

SEQ ID NO: 20 - protein, 18 aa, synthetic construct
EAKKESEKLIKLETDPAM

SEQ ID NO: 21 - protein, 21 aa, synthetic construct
PEVEAVHEKALAVAPKLTEAQ

SEQ ID NO: 22 - protein, 228 aa, synthetic construct
XXXXXVEQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGKALVKGKPDEVREKAKKFVKKIRGCTEGTXXXXXXXXXXXXXXXXMKMEELFKKHKIVAVLRANSVEEAIEKAVAVFAGGVHLIEITFTVPDADTVIKALSVLKEKGAIIGAGTVTS

SEQ ID NO: 23 - protein, 229 aa, synthetic construct
XXXXXXYMPGVMTPTELVKAMKLGHDILKLFPGEVVGPQFVKAMKGPFPNVKFVPTGGVNLDNVCKWFKAGVLAVGVGKALVKGKPDEVREKAKKFVKKIRGCTEXXXXXXXXXXXXXXXXXXMKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVF

SEQ ID NO: 24 - protein, 231 aa, synthetic construct
XXXXXXXXLKLFPGEVVGPQFVKAMKGPFPNVKFVPTGGVNLDNVCKWFKAGVLAVGVGKALVKGKPDEVREKAKKFVKKIRGCTEXXXXXXXXXXXXXXXXXXMKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDI

SEQ ID NO: 25 - protein, 228 aa, synthetic construct
XXXXXXXMKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGKALVKGKPDEVREKAKKFVKKIRGCTEXXXXXXXXXXXXXXXX

SEQ ID NO: 26 - protein, 156 aa, synthetic construct
NQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAA

SEQ ID NO: 27 - protein, 156 aa, synthetic construct
NQHSHKDYETVRIAVVRARWHAEIVDACVSAFEAAMADIGGDRFAVDVFDVPGAYEIPLHARTLAETGRYGAVLGTAFVVNGGIYRHEFVASAVIDGMMNVQLSTGVPVLSAVLTPHRYRDSDAHTLLFLALFAVKGMEAARACVEILAAREKIAA

SEQ ID NO: 28 - protein, 272 aa, synthetic construct
MKKYLLGIGLILALIACKQNVSSLDEKNSVSVDVPGGMKVLVSKEKNKDGKYDLMATVDNVDLKGTSDKNNGSGILEGVKADKSKVKLTVADDLSKTTLEVLKEDGTVVSRKVTSKDKSTTEAKFNEKGELSEKTMTRANGTTLEYSQMTNEDNAAKAVETLKNGIKFEGNLASGKTAVEIKEGTVTLKREIDKNGKVTVSLNDTASGSKKTASWQESTSTLTISANSKKTKDLVFLTNGTITVQNYDSAGTKLEGSAAEIKKLDELKNALR

SEQ ID NO: 29 - protein, 295 aa, synthetic construct
MKQYLLGFTLVFALIACAQKGANPEQKGGDADVLNTSKTEKYLNKNLPSEAEDLVSLFNDSEIFVSKEKNKDGKYVLRAIVDTVELKGVADKNDGSEGKLEGLKPDNSKVTMSISKDQNTITIETRDSSNTKVASKVFKKDGSLTEESYKAGQLDSKKLTRSNKTTLEYSDMTNAENATTAIETLKNGIEFKGSLVGGKATLQIVESTVTLTREIDKDGKLKIYLKDTASSSKKTVSWNDTDTLTISAEGKKTKDLVFLTDGTITVQNYDSASGTTLEGTATEIKNLEALKTALK

SEQ ID NO: 30 - protein, 274 aa, synthetic construct
MNRTAFCCLSLTTALILTACSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 31 - protein, 298 aa, synthetic construct
MSKNKDQRTAKTLERTWDTLNHLLFISSCLYKLNLKSVAQITLSILAMIISTSLIIAAIIFIASANHKVTPTTAIIQDATSQIKNTTPTYLTQNPQLGISPSNPSEITSQITTILASTTPGVKSTLQSTTVKTKNTTTTQTQPSKPTTKQRQNKPPSKPNNDFHFEVFNFVPCSICSNNPTCWAICKRIPNKKPGKKTTTKPTKKPTLKTTKKDPKPQTTKSKEVPTTKPTEEPTINTTKTNIITTLLTSNTTGNPELTSQMETFHSTSSEGNPSPSQVSTTSEYPSQPSSPPNTPRQ

SEQ ID NO: 32 - protein, 219 aa, synthetic construct
MEVKVENIRAIDMLKARVKNRVARSKCFKNASLILIGITTLSIALNIYLIINYTIQKTSSESEHHTSSPPTESNKEASTISTDNPDINPNSQHPTQQSTENPTLNPAASVSPSETEPASTPDTTNRLSSVDRSTAQPSESRTKTKPTVHTRNNPSTASSTQSPPRATTKAIRRATTFRMSSTGKRPTTTSVQSDSSTTTQNHEETGSANPQASVSTMQN

SEQ ID NO: 33 - protein, 16 aa, synthetic construct
ENHARFAALRAELAGT

SEQ ID NO: 34 - protein, 16 aa, synthetic construct
ENPELTKEVAAFLAGT

SEQ ID NO: 35 - protein, 16 aa, synthetic construct
EYSEQFEARKKKLEGT

SEQ ID NO: 36 - protein, 16 aa, synthetic construct
RIDEEYQKRLEKLRGT

SEQ ID NO: 37 - protein, 16 aa, synthetic construct
KYKEQLDKQLKLQLGT

SEQ ID NO: 38 - protein, 16 aa, synthetic construct
KEKEYFEEQLEKLKGT

SEQ ID NO: 39 - protein, 16 aa, synthetic construct
YSKARLAEIKKALAGT

SEQ ID NO: 40 - protein, 16 aa, synthetic construct
AADEHMAAIMAALKGT

SEQ ID NO: 41 - protein, 16 aa, synthetic construct
VGDKLLAELKAQLAGT

SEQ ID NO: 42 - protein, 16 aa, synthetic construct
YLKANAEKLHKLLAGT

SEQ ID NO: 43 - protein, 16 aa, synthetic construct
ILAKLKAKILAKLKGT

SEQ ID NO: 44 - protein, 16 aa, synthetic construct
YSQATLKEILKALAGT

SEQ ID NO: 45 - protein, 21 aa, synthetic construct
FSKEACKKAILETKDGSGSGT

SEQ ID NO: 46 - protein, 18 aa, synthetic construct
PEVQAVHKKALAVAPKGT

SEQ ID NO: 47 - protein, 24 aa, synthetic construct
EEVKAVQQKALALAPKLTGSGSGT

SEQ ID NO: 48 - protein, 21 aa, synthetic construct
AEVAANQAKALSLAPPEAGGT

SEQ ID NO: 49 - protein, 24 aa, synthetic construct
AEVEANQAKALSLAPAPAGSGSGT

SEQ ID NO: 50 - protein, 24 aa, synthetic construct
EEVEAVQKKALSIAEELEGSGSGT

SEQ ID NO: 51 - protein, 21 aa, synthetic construct
EEVAAVQKKALSLAPKEPSGT

SEQ ID NO: 52 - protein, 12 aa, synthetic construct
DTLALLKERKGT

SEQ ID NO: 53 - protein, 12 aa, synthetic construct
DSMAILEKVKGT

SEQ ID NO: 54 - protein, 12 aa, synthetic construct
NQMELVKKVFGT

SEQ ID NO: 55 - protein, 12 aa, synthetic construct
NAMELVKKALGT

SEQ ID NO: 56 - protein, 12 aa, synthetic construct
NQMELVKKAEGT

SEQ ID NO: 57 - protein, 12 aa, synthetic construct
DSMELFKKAEGT

SEQ ID NO: 58 - protein, 15 aa, synthetic construct
DAMELLKEAEAIMGT

SEQ ID NO: 59 - protein, 15 aa, synthetic construct
DPMKLVEEVEKLLGT

SEQ ID NO: 60 - protein, 15 aa, synthetic construct
DPMAEVEKAKALEGT

SEQ ID NO: 61 - protein, 15 aa, synthetic construct
DPMALVDKVLALFGT

SEQ ID NO: 62 - protein, 207 aa, synthetic construct
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGKALVKGKPDEVREKAKKFVKKIRGCTEGT

SEQ ID NO: 63 - protein, 231 aa, synthetic construct
MGSGSVEQCRKAVEAGAEYIVSPHLDEEISQFCKEKGIPYMPGVMTPTELVKAMKLGHLLLKLFPGEVVGPQFVKAMKKTFPKARFVPTGGVNLDNVCEWFKAGVLAVGVGSALVKGTPDEVREKAKAFVEEAEKCSEENKKVGEELIKLVTRPEDREMVEIFYKEKIVAVLRANSVEEAIEKAVAVFAGGVTIIEITFTVPDADTVIKALSVLKEKGAVIGAGTVTGSGT

SEQ ID NO: 64 - protein, 231 aa, synthetic construct
MGSIPYMPGVMTPTELVKAMKLGHLLLKLFPGEVVGPQFVKAMKKTFPKARFVPTGGVNLDNVCEWFKAGVLAVGVGSALVKGTPDEVREKAKAFVEEAAKCSEENKKVGEELIKLVTRPEDREMVEIFYEKKIVAVLRANSVEEAIEKAVAVFAGGVTIIEITFTVPDADTVIKALSVLKEKGAVIGAGTVTSVEQCRKAVEAGAEYIVSPHLDEEISQFCKEKGSGSGT

SEQ ID NO: 65 - protein, 232 aa, synthetic construct
MGSGHLLLKLFPGEVVGPQFVKAMKKTFPKARFVPTGGVNLDNVCEWFKAGVLAVGVGSALVKGTPDEVREKAKAFVEEAAKCSEENKKVGEELIKLVTRPEDREMVEIFYKEKIVAVLRANSVEEAIEKAVAVFAGGVTIIEITFTVPDADTVIKALSVLKEKGAVIGAGTVTSVEQCRKAVEAGAEYIVSPHLDEEISQFCKEKGIPYMPGVMTPTELVKAMKLGSGSGT

SEQ ID NO: 66 - protein, 227 aa, synthetic construct
MGAPADRELLRKLLENRIVAVLRANSVEEAIEKAVAVFAGGVTIIEITFTVPDADTVIKALSVLKEKGAVIGAGTVTSVEQCRKAVEAGAEYIVSPHLDEEISQFCKEKGIPYMPGVMTPTELVKAMKLGHLLLKLFPGEVVGPQFVKAMKKTYPEARFVPTGGVNLDNVCEWIKAGAIAVGVGSALVKGTPDEVREKAKAFVEEAAKCAAENHARFAALRAELAGT

SEQ ID NO: 67 - protein, 227 aa, synthetic construct
MGAPEEKKMIALLAENPIVAVLRANSVEEAIEKAVAVFAGGVTIIEITFTVPDADTVIKALSVLKEKGAVIGAGTVTSVEQCRKAVEAGAEYIVSPHLDEEISQFCKEKGIPYMPGVMTPTELVKAMKLGHLLLKLFPGEVVGPQFVKAMKKTYPEARFVPTGGVNLDNVCEWIKAGALAVGVGSALVKGTPDEVREKAKAFVEEALKCRGENPELTKEVAAFLAGT

SEQ ID NO: 68 - protein, 231 aa, synthetic construct
MGSGSVEQCRKAVEAGAEYIVSPHLDEEISQFCKEKGVPYMPGVMTPTELVKAMKLGHLLLKLFPGEVVGPQFVKAMKKTFPDAAFVPTGGVNLDNVCEWFKAGVLAVGVGSALVKGTPDEVREKAKAFVEKAKKCDEVNKEVGKKLLLLVTDPADKKMVERFYKEKIVAVLRANSVEEAIEKAVAVFAGGVTIIEITFTVPDADTVIKALSVLKEKGAIIGAGTVTGSGT

SEQ ID NO: 69 - protein, 232 aa, synthetic construct
MGSGVPYMPGVMTPTELVKAMKLGHLLLKLFPGEVVGPQFVKAMKKTFPDAAFVPTGGVNLDNVCEWFKAGVLAVGVGSALVKGTPDEVREKAKAFVEKAKKCDQVNNEVGKKLLLLVTDPADKKMVERFYEEKIVAVLRANSVEEAIEKAVAVFAGGVTIIEITFTVPDADTVIKALSVLKEKGAIIGAGTVTSVEQCRKAVEAGAEYIVSPHLDEEISQFCKEKGSGSGT

SEQ ID NO: 70 - protein, 227 aa, synthetic construct
MGDPKELAMLKAFLEEKIVAVLRANSVEEAIEKAVAVFAGGVKIIEITFTVPDADTVIKALSVLKEKGAIIGAGTVTSVEQCRKAVEAGAEYIVSPHLDEEISQFCKEKGVPYMPGVMTPTELVKAMKLGHQLLKLFPGEVVGPQFVKAMKKTYPDAAFVPTGGVNLDNVCEWFKAGATAVGVGSALVKGTPDEVREKAKAFVEKAAKCEGEYSEQFEARKKKLEGT

SEQ ID NO: 71 - protein, 226 aa, synthetic construct
MPEKEREIMIAFLKNRIVAVLRANSVEEAIEKAVAVFAGGVKIIEITFTVPDADTVIKALSVLKEKGAIIGAGTVTSVEQCRKAVEAGAEYIVSPHLDEEISQFCKEKGVPYMPGVMTPTELVKAMKLGHRLLKLFPGEVVGPQFVKAMKKTYPDAAFVPTGGVNLDNVCEWFDAGAVAVGVGSALVKGTPDEVREKAKAFVEKAAKCRARIDEEYQKRLEKLRGT

SEQ ID NO: 72 - protein, 227 aa, synthetic construct
MGSEADLKMLKKLYEEKIVAVLRANSVEEAIEKAVAVFAGGVKIIEITFTVPDADTVIKALSVLKEKGAIIGAGTVTSVEQCRKAVEAGAEYIVSPHLDEEISQFCKEKGVPYMPGVMTPTELVKAMKLGHKLLKLFPGEVVGPQFVKAMKKTYPEAAFVPTGGVNLDNVCEWIEAGAVAVGVGSALVKGTPDEVREKAKAFVEKANECAGKYKEQLDKQLKLQLGT

SEQ ID NO: 73 - protein, 228 aa, synthetic construct
MGLPEVELKMIEKIMEEGIVAVLRANSVEEAIEKAVAVFAGGVKIIEITFTVPDADTVIKALSVLKEKGAIIGAGTVTSVEQCRKAVEAGAEYIVSPHLDEEISQFCKEKGVPYMPGVMTPTELVKAMKLGHVLLKLFPGEVVGPQFVKAMKKTYPNVAFVPTGGVNLDNVCEWIEAGAAAVGVGSALVKGTPDEVREKAKAFVEKANECRAKEKEYFEEQLEKLKGT

SEQ ID NO: 74 - protein, 228 aa, synthetic construct
MGVDEKDLKLLEALAANRIVAVLRANSVEEAIEKAVAVFAGGVTIIEITFTVPDADTVIKALSVLKEKGAIIGAGTVTSVEQCRKAVEAGAEYIVSPHLDEEISQFCKEKGVPYMPGVMTPTELVKAMKLGHLLLKLFPGEVVGPQFVKAMKKTYPTAAFVPTGGVNLDNVCEWLKAGAVAVGVGSALVKGTPDEVREKAKAFVAKADEYAKYSKARLAEIKKALAGT

SEQ ID NO: 75 - protein, 228 aa, synthetic construct
MGVSEKEIEMLKKFNEARIVAVLRANSVEEAIEKAVAVFAGGVTIIEITFTVPDADTVIKALSVLKEKGAIIGAGTVTSVEQCRKAVEAGAEYIVSPHLDEEISQFCKEKGVPYMPGVMTPTELVKAMKLGHLLLKLFPGEVVGPQFVKAMKKTYPLAAFVPTGGVNLDNVCEWLEAGCIAVGVGSALVKGTPDEVREKAKAFVAKARECAAAADEHMAAIMAALKGT

SEQ ID NO: 76 - protein, 228 aa, synthetic construct
MGLSPAEQAMLLAVVENRIVAVLRANSVEEAIEKAVAVFAGGVTIIEITFTVPDADTVIKALSVLKEKGAIIGAGTVTSVEQCRKAVEAGAEYIVSPHLDEEISQFCKEKGVPYMPGVMTPTELVKAMKLGHLLLKLFPGEVVGPQFVKAMKKTYPGVAFVPTGGVNLDNVCEWLEAGAAAVGVGSALVKGTPDEVREKAKAFVAKADEMGAVGDKLLAELKAQLAGT

SEQ ID NO: 77 - protein, 228 aa, synthetic construct
MGYPEAQIELLDKVIKEGIVAVLRANSVEEAIEKAVAVFAGGVTIIEITFTVPDADTVIKALSVLKEKGAIIGAGTVTSVEQCRKAVEAGAEYIVSPHLDEEISQFCKEKGVPYMPGVMTPTELVKAMKLGHLLLKLFPGEVVGPQFVKAMKKTYPGAAFVPTGGVNLDNVCEWLKAGAAAVGVGSALVKGTPDEVREKAKAFVAKAKECSKYLKANAEKLHKLLAGT

SEQ ID NO: 78 - protein, 228 aa, synthetic construct
MGLSEKEIAIIEAFLENPIVAVLRANSVEEAIEKAVAVFAGGVTIIEITFTVPDADTVIKALSVLKEKGAIIGAGTVTSVEQCRKAVEAGAEYIVSPHLDEEISQFCKEKGVPYMPGVMTPTELVKAMKLGHLLLKLFPGEVVGPQFVKAMKKTYPDVAFVPTGGVNLDNVCEWLEAGAVAVGVGSALVKGTPDEVREKAKAFVAKAGEKAAILAKLKAKILAKLKGT

SEQ ID NO: 79 - protein, 228 aa, synthetic construct
MGVSEADLALLKALAENQIVAVLRANSVEEAIEKAVAVFAGGVTIIEITFTVPDADTVIKALSVLKEKGAIIGAGTVTSVEQCRKAVEAGAEYIVSPHLDEEISQFCKEKGVPYMPGVMTPTELVKAMKLGHLLLKLFPGEVVGPQFVKAMKKTYPSAAFVPTGGVNLDNVCEWLKAGCIAVGVGSALVKGTPDEVREKAKAFVAKADECAKYSQATLKEILKALAGT

SEQ ID NO: 80 - protein, 231 aa, synthetic construct
MGSGSVEQCRKAVEAGAEYIVSPHLDEEISQFCKEKGIPYMPGVMTPTELVKAMKLGHRVLKLFPGEVVGPQFVKAMKKTFPDARFVPTGGVNLDNVCEWFKAGVLAVGVGSALVKGTPDEVREKAKAFVEKAAECSEENEKEGKKALKLETDPAMKKMVKIFYKEKIVAVLRANSVEEAIEKAVAVFAGGVTVIEITFTVPDADTVIKALSVLKEKGAVIGAGTVTGSGT

SEQ ID NO: 81 - protein, 232 aa, synthetic construct
MGSGIPYMPGVMTPTELVKAMKLGHRVLKLFPGEVVGPQFVKAMKKTFPDARFVPTGGVNLDNVCEWFKAGVLAVGVGSALVKGTPDEVREKAKAFVEKAAECSEENEKEGKKALKLETDPAMKKMVKIFYKEKIVAVLRANSVEEAIEKAVAVFAGGVTVIEITFTVPDADTVIKALSVLKEKGAVIGAGTVTSVEQCRKAVEAGAEYIVSPHLDEEISQFCKEKGSGSGT

SEQ ID NO: 82 - protein, 233 aa, synthetic construct
MGSGGHRVLKLFPGEVVGPQFVKAMKKTFPDARFVPTGGVNLDNVCEWFKAGVLAVGVGSALVKGTPDEVREKAKAFVEKAAECSEENEKEGKKALKLETDPAMKKMVKIFYKEKIVAVLRANSVEEAIEKAVAVFAGGVTVIEITFTVPDADTVIKALSVLKEKGAVIGAGTVTSVEQCRKAVEAGAEYIVSPHLDEEISQFCKEKGIPYMPGVMTPTELVKAMKLGSGSGT

SEQ ID NO: 83 - protein, 231 aa, synthetic construct
MGSGSVEQCRKAVEAGAEYIVSPHLDEEISQFCKEKGIPYMPGVMTPTELVKAMKLGHKLLKLFPGEVVGPQFVKAMKKTFPDAAFVPTGGVNLDNVCEWFKAGVLAVGVGSALVKGTPDEVREKAKAFVEKAKECSEEADKESEKLIKLETDPAMLRMAKIFGKEKIVAVLRANSVEEAIEKAVAVFAGGVTVIEITFTVPDADTVIKALSVLKEKGAVIGAGTVTGSGT

SEQ ID NO: 84 - protein, 232 aa, synthetic construct
MGSGIPYMPGVMTPTELVKAMKLGHKLLKLFPGEVVGPQFVKAMKKTFPDAAFVPTGGVNLDNVCEWFKAGVLAVGVGSALVKGTPDEVREKAKAFVEKAEECSSEAKKESEKLIKLETDPAMLRMAKIFGKEKIVAVLRANSVEEAIEKAVAVFAGGVTVIEITFTVPDADTVIKALSVLKEKGAVIGAGTVTSVEQCRKAVEAGAEYIVSPHLDEEISQFCKEKGSGSGT

SEQ ID NO: 85 - protein, 233 aa, synthetic construct
MGSGGHKLLKLFPGEVVGPQFVKAMKKTFPDAAFVPTGGVNLDNVCEWFKAGVLAVGVGSALVKGTPDEVREKAKAFVEKAEECSEEAKKESEKLIKLETDPAMLRMAKIFGKEKIVAVLRANSVEEAIEKAVAVFAGGVTVIEITFTVPDADTVIKALSVLKEKGAVIGAGTVTSVEQCRKAVEAGAEYIVSPHLDEEISQFCKEKGIPYMPGVMTPTELVKAMKLGSGSGT

SEQ ID NO: 86 - protein, 232 aa, synthetic construct
MGDKAMASMAKQFCKNKIVAVLRANSVEEAIEKAVAVFAGGVAIIEITFTVPDADTVIKALSVLKEKGASIGAGTVTSVEQCRKAVEAGADYIVSPHLDEEISQFCKEKGVPYMPGVMTPTELVKAMKLGHVILKLFPGEVVGPQFVKAMKKTFPNAQFVPTGGVNLDNVCEWFKAGVLAVGVGSALVKGTPDEVREKAKAFVEKVKECAHFSKEACKKAILETKDGSGSGT

SEQ ID NO: 87 - protein, 234 aa, synthetic construct
MGSGSVEQCRKAVEAGADYIVSPHLDEEISQFCKEKGVAYMPGVMTPTELVKAMKLGHLVLKLFPGEVVGPQFVKAMKKTFPDVFFVPTGGVNLDNVCEWFKAGVLAVGVGSALVKGTPDEVREKAKAFVDKVIACYGPEVQAVHKKALAVAPKLTEAQALMLKAFVEEKIVAVLRANSVEEAIEKAVAVFAGGVNIIEITFTVPDADTVIKALSVLKEKGAIIGAGTVTGSGT

SEQ ID NO: 88 - protein, 235 aa, synthetic construct
MGSGVAYMPGVMTPTELVKAMKLGHLVLKLFPGEVVGPQFVKAMKKTFPDVFFVPTGGVNLDNVCEWFKAGVLAVGVGSALVKGTPDEVREKAKAFVDKVTACYGPEVEAVHEKALAVAPKLTEAQALMLKAFVEEKIVAVLRANSVEEAIEKAVAVFAGGVNIIEITFTVPDADTVIKALSVLKEKGAIIGAGTVTSVEQCRKAVEAGADYIVSPHLDEEISQFCKEKGSGSGT

SEQ ID NO: 89 - protein, 236 aa, synthetic construct
MGSSGHLVLKLFPGEVVGPQFVKAMKKTFPDVFFVPTGGVNLDNVCEWFKAGVLAVGVGSALVKGTPDEVREKAKAFVDKVTACYGPEVEAVHEKALAVAPKLTEAQALMLKAFVEEKIVAVLRANSVEEAIEKAVAVFAGGVNIIEITFTVPDADTVIKALSVLKEKGAIIGAGTVTSVEQCRKAVEAGADYIVSPHLDEEISQFCKEVGVAYMPGVMTPTELVKAMKLGSGSGT

SEQ ID NO: 90 - protein, 228 aa, synthetic construct
MTEAQALMLKAFVEEKIVAVLRANSVEEAIEKAVAVFAGGVNIIEITFTVPDADTVIKALSVLKEKGAIIGAGTVTSVEQCRKAVEAGADYIVSPHLDEEISQFCKEVGVAYMPGVMTPTELVKAMKLGHLVLKLFPGEVVGPQFVKAMKKTFPDVFFVPTGGVNLDNVCEWFKAGVLAVGVGSALVKGTPDEVREKAKAFVEKVLACIGPEVQAVHKKALAVAPKGT

SEQ ID NO: 91 - protein, 235 aa, synthetic construct
MGTEANALMLKRFVEEKIVAVLRANSVEEAIEKAVAVFAGGVNIIEITFTVPDADTVIKALSVLKEKGAVIGAGTVTSVEQCRKAVEAGAAYIVSPHLDEEISQFCKEVGVAYMPGVMTPTELVKAMKLGHLVLKLFPGEVVGPQFVKAMKKTFPDAVFVPTGGVNLDNVCEWFKAGVLAVGVGSALVKGTPDEVREKAKAFREKVAACDGEEVKAVQQKALALAPKLTGSGSGT

SEQ ID NO: 92 - protein, 231 aa, synthetic construct
MTPAEALMLKRFVKEKIVAVLRANSVEEAIEKAVAVFAGGVNIIEITFTVPDADTVIKALSVLKEKGAIIGAGTVTSVEQCRKAVEAGADYIVSPHLDEEISQFCKEVGVAYMPGVMTPTELVKAMKLGHIILKLFPGEVVGPQFVKAMKKTFPDAVFVPTGGVNLDNVCEWFKAGVVAVGVGSALVKGTPDEVREKAKAFREKVATCVGAEVAANQAKALSLAPPEAGGT

SEQ ID NO: 93 - protein, 234 aa, synthetic construct
MTPAEALMLKRFVKEKIVAVLRANSVEEAIEKAVAVFAGGVNIIEITFTVPDADTVIKALSVLKEKGAIIGAGTVTSVEQCRKAVEAGADYIVSPHLDEEISQFCKEVGVAYMPGVMTPTELVKAMKLGHIILKLFPGEVVGPQFVKAMKKTFPDAVFVPTGGVNLDNVCEWFKAGVVAVGVGSALVKGTPDEVREKAKAFREKVATCVGAEVEANQAKALSLAPAPAGSGSGT

SEQ ID NO: 94 - protein, 234 aa, synthetic construct
MTPAEALMLKRFVEEKIVAVLRANSVEEAIEKAVAVFAGGVNIIEITFTVPDADTVIKALSVLKEKGAVIGAGTVTSVEQCRKAVEAGADYIVSPHLDEEISQFCKEVGVAYMPGVMTPTELVKAMKLGHILLKLFPGEVVGPQFVKAMKETFPDAFFVPTGGVNLDNVCEWFKAGVVAVGVGSALVKGTPDEVREKAKAFVEKVNSCTGEEVEAVQKKALSIAEELEGSGSGT

SEQ ID NO: 95 - protein, 231 aa, synthetic construct
MTPAEALMLKRFVEEKIVAVLRANSVEEAIEKAVAVFAGGVNIIEITFTVPDADTVIKALSVLKEKGAVIGAGTVTSVEQCRKAVEAGADYIVSPHLDEEISQFCKEVGVAYMPGVMTPTELVKAMKLGHILLKLFPGEVVGPQFVKAMKETFPDAFFVPTGGVNLDNVCEWFKAGVVAVGVGSALVKGTPDEVREKAKAFKAKVASCTGEEVAAVQKKALSLAPKEPSGT

SEQ ID NO: 96 - protein, 221 aa, synthetic construct
MGNKEEIEEKFAREKIVAVLRANSVEEAIEKAVAVFAGGVGIIEITFTVPDADTVIKALSVLKEKGASIGAGTVTSVEQCRKAVEAGAEYIVSPHLDEEISQFCKELGIPYMPGVMTPTELVKAMKLGHKVLKLFPGEVVGPQFVKAMKKTYPDVLFVPTGGVNLDNVCEWLKAGALAVGVGSALVKGTPDEVREKAKAFVEKVKACGVDTLALLKERKGT

SEQ ID NO: 97 - protein, 220 aa, synthetic construct
MGNEEIEEKFAKEKIVAVLRANSVEEAIEKAVAVFAGGVGIIEITFTVPDADTVIKALSVLKEKGASIGAGTVTSVEQCRKAVEAGAEYIVSPHLDEEISQFCKELGIPYMPGVMTPTELVKAMKLGHKVLKLFPGEVVGPQFVKAMKKTYPEALFVPTGGVNLDNVCEWLKAGAIAVGVGSALVKGTPDEVREKAKAFVEKVKACGVDSMAILEKVKGT

SEQ ID NO: 98 - protein, 220 aa, synthetic construct
MGNKEIIEKFAKEKIVAVLRANSVEEAIEKAVAVFAGGVGIIEITFTVPDADTVIKALSVLKEKGASIGAGTVTSVEQCRKAVEAGAEYIVSPHLDEEISQFCKELGIPYMPGVMTPTELVKAMKLGHKVLKLFPGEVVGPQFVKAMKKTYPDALFVPTGGVNLDNVCEWLKAGALAVGVGSALVKGTPDEVREKAKAFVEKVKACGVNQMELVKKVFGT

SEQ ID NO: 99 - protein, 220 aa, synthetic construct
MGNVEIIEKFAKEKIVAVLRANSVEEAIEKAVAVFAGGVGIIEITFTVPDADTVIKALSVLKEKGASIGAGTVTSVEQCRKAVEAGAEYIVSPHLDEEISQFCKELGIPYMPGVMTPTELVKAMKLGHKVLKLFPGEVVGPQFVKAMKKTYPDALFVPTGGVNLDNVCEWLKAGALAVGVGSALVKGTPDEVREKAKAFVEKVRASGVNAMELVKKALGT

SEQ ID NO: 100 - protein, 221 aa, synthetic construct
MGNPKEIIEKFAKEKIVAVLRANSVEEAIEKAVAVFAGGVGIIEITFTVPDADTVIKALSVLKEKGASIGAGTVTSVEQCRKAVEAGAEYIVSPHLDEEISQFCKELGIPYMPGVMTPTELVKAMKLGHKVLKLFPGEVVGPQFVKAMKKTYPEALFVPTGGVNLDNVCEWFKAGALAVGVGSALVKGTPDEVREKAKAFVEKVKACGVNQMELVKKAEGT

SEQ ID NO: 101 - protein, 220 aa, synthetic construct
MGNKEIGEKFAREKIVAVLRANSVEEAIEKAVAVFAGGVGIIEITFTVPDADTVIKALSVLKEKGASIGAGTVTSVEQCRKAVEAGAEYIVSPHLDEEISQFCKELGIPYMPGVMTPTELVKAMKLGHKVLKLFPGEVVGPQFVKAMKKTYPEALFVPTGGVNLDNVCEWLEAGALAVGVGSALVKGTPDEVREKAKAFVEKVKACPFDSMELFKKAEGT

SEQ ID NO: 102 - protein, 223 aa, synthetic construct
MGDLKMAKAFAREKIVAVLRANSVEEAIEKAVAVFAGGVGIIEITFTVPDADTVIKALSVLKEKGAKIGAGTVTSVEQCRKAVEAGADYIVSPHLDEEISQFCKELGIPYMPGVMTPTELVKAMKLGHTILKLFPGEVVGPQFVKAMKKTYPEALFVPTGGVNLDNVCEWLEAGALAVGVGSALVKGTPDEVREKAKAFVAKVAACPVDAMELLKEAEAIMGT

SEQ ID NO: 103 - protein, 223 aa, synthetic construct
MGDKKMAKAFAKEKIVAVLRANSVEEAIEKAVAVFAGGVGIIEITFTVPDADTVIKALSVLKEKGAKIGAGTVTSVEQCRKAVEAGADYIVSPHLDEEISQFCKELGIPYMPGVMTPTELVKAMKLGHTILKLFPGEVVGPQFVKAMKKTYPQALFVPTGGVNLDNVCEWLEAGALAVGVGSALVKGTPDEVREKAKAFVAKVAACPYDPMKLVEEVEKLLGT

SEQ ID NO: 104 - protein, 224 aa, synthetic construct
MGTDEKMAKAFAREKIVAVLRANSVEEAIEKAVAVFAGGVGIIEITFTVPDADTVIKALSVLKEKGAKIGAGTVTSVEQCRKAVEAGADYIVSPHLDEEISQFCKELGIPYMPGVMTPTELVKAMKLGHTILKLFPGEVVGPQFVKAMKKTYPQALFVPTGGVNLDNVCEWLKAGAIAVGVGSALVKGTPDEVREKAKAFVAKVAACGVDPMAEVEKAKALEGT

SEQ ID NO: 105 - protein, 223 aa, synthetic construct
MGDEKMAKAFAREKIVAVLRANSVEEAIEKAVAVFAGGVGIIEITFTVPDADTVIKALSVLKEKGAKIGAGTVTSVEQCRKAVEAGADYIVSPHLDEEISQFCKELGIPYMPGVMTPTELVKAMKLGHTILKLFPGEVVGPQFVKAMKKTYPEALFVPTGGVNLDNVCEWLDAGALAVGVGSALVKGTPDEVREKAKAFVAKVAACPFDPMALVDKVLALFGT

SEQ ID NO: 106 - protein, 17 aa, synthetic construct
KEVGKKLLLLVTDPADK

SEQ ID NO: 107 - protein, 17 aa, synthetic construct
DKESEKLIKLETDPAML

SEQ ID NO: 108 - protein, 17 aa, synthetic construct
EKEGKKALKLETDPAMK

SEQ ID NO: 109 - protein, 20 aa, synthetic construct
VQAVHKKALAVAPKLTEAQA

SEQ ID NO: 110 - protein, 19 aa, synthetic construct
ENKKVGEELIKLVTRPEDR

SEQ ID NO: 111 - protein, 19 aa, synthetic construct
VNNEVGKKLLLLVTDPADK

SEQ ID NO: 112 - protein, 19 aa, synthetic construct
ENEKEGKKALKLETDPAMK

SEQ ID NO: 113 - protein, 19 aa, synthetic construct
EAKKESEKLIKLETDPAML

SEQ ID NO: 114 - protein, 22 aa, synthetic construct
PEVEAVHEKALAVAPKLTEAQA

SEQ ID NO: 115 - protein, 238 aa, synthetic construct
MGSSHHHHHHGSGSEADLKMLKKLYEERIVAVLRANSVEEAIEKAVAVFAGGVKIIEITFTVPDADTVIKALSVLKEKGAIIGAGTVTSVEQCRKAVEAGAEYIVSPHLDEEISQFCKEKGVPYMPGVMTPTELVKAMKLGHKLLKLFPGEVVGPQFVKAMKKTYPEAAFVPTGGVNLDNVCEWIEAGAVAVGVGSALVKGTPDEVREKAKAFVEKARLCAGKYKEQLDKQLALQLGT

SEQ ID NO: 116 - protein, 238 aa, synthetic construct
MGSSHHHHHHGSGSEADLKMLKKLYQERIVAVLRANSVEEAIEKAVAVFAGGVKIIEITFTVPDADTVIKALSVLKEKGAIIGAGTVTSVEQCRKAVEAGAEYIVSPHLDEEISQFCKEKGVPYMPGVMTPTELVKAMKLGHKLLKLFPGEVVGPQFVKAMKKTYPEAAFVPTGGVNLDNVCEWIEAGAVAVGVGSALVKGTPDEVREKAKAFVEKANLCAGKYKKQLDKQLALQLGT

SEQ ID NO: 117 - protein, 238 aa, synthetic construct
MGSSHHHHHHGSGSEADLKMLKKLYTEKIVAVLRANSVEEAIEKAVAVFAGGVKIIEITFTVPDADTVIKALSVLKEKGAIIGAGTVTSVEQCRKAVEAGAEYIVSPHLDEEISQFCKEKGVPYMPGVMTPTELVKAMKLGHKLLKLFPGEVVGPQFVKAMKKTYPEAAFVPTGGVNLDNVCEWIEAGAVAVGVGSALVKGTPDEVREKAKAFVQKANECAGKYSDQLDKQLKLQLGT

SEQ ID NO: 118 - protein, 238 aa, synthetic construct
MGSSHHHHHHGSGSEADLKMLKKLYTEKIVAVLRANSVEEAIEKAVAVFAGGVKIIEITFTVPDADTVIKALSVLKEKGAIIGAGTVTSVEQCRKAVEAGAEYIVSPHLDEEISQFCKEKGVPYMPGVMTPTELVKAMKLGHKLLKLFPGEVVGPQFVKAMKKTYPEAAFVPTGGVNLDNVCEWIEAGAVAVGVGSALVKGTPDEVREKAKAFVQKANECAGKYSSQLDKQLSLQLGT

SEQ ID NO: 119 - protein, 242 aa, synthetic construct
MGSSHHHHHHGSGGHKLLKLFPGEVVGPQFVKAMKKTFPDAAFVPTGGVNLDNVCEWFKAGVLAVGVGSALVKGTPDEVREKAKAFVEKANRCSEEAEKESEKLIKLETDPAMLRMAKIFGKEKIVAVLRANSVEEAIEKAVAVFAGGVTVIEITFTVPDADTVIKALSVLKEKGAVIGAGTVTSVEQCRKAVEAGAEYIVSPHLDEEISQFCKEKGIPYMPGVMTPTELVKAMKLGSGSGT

SEQ ID NO: 120 - protein, 239 aa, synthetic construct
MGSSHHHHHHGSTEAQALMLKAFVEEKIVAVLRANSVEEAIEKAVAVFAGGVNIIEITFTVPDADTVIKALSVLKEKGAIIGAGTVTSVEQCRKAVEAGADYIVSPHLDEEISQFCKEVGVAYMPGVMTPTELVKAMKLGHLVLKLFPGEVVGPQFVKAMKKTFPDVFFVPTGGVNLDNVCEWFKAGVLAVGVGSALVKGTPDEVREKAKAFVDKVTACIDAEVNAVHKKALAVAPKGT

SEQ ID NO: 121 - protein, 233 aa, synthetic construct
MGSSHHHHHHGSGSGNEEIEEKFASEKIVAVLRANSVEEAIEKAVAVFAGGVGIIEITFTVPDADTVIKALSVLKEKGASIGAGTVTSVEQCRKAVEAGAEYIVSPHLDEEISQFCKELGIPYMPGVMTPTELVKAMKLGHKVLKLFPGEVVGPQFVKAMKKTYPEALFVPTGGVNLDNVCEWLKAGAIAVGVGSALVKGTPDEVREKAKAFVEKVSACGVDSMAILSKVKGT

SEQ ID NO: 122 - protein, 233 aa, synthetic construct
MGSSHHHHHHGSGSGNEEIEEKFAKEKIVAVLRANSVEEAIEKAVAVFAGGVGIIEITFTVPDADTVIKALSVLKEKGASIGAGTVTSVEQCRKAVEAGAEYIVSPHLDEEISQFCKELGIPYMPGVMTPTELVKAMKLGHKVLKLFPGEVVGPQFVKAMKKTYPEALFVPTGGVNLDNVCEWLKAGAIAVGVGSALVKGTPDEVREKAKAFVEKVSACGVDSMAILEKVKGT

SEQ ID NO: 123 - protein, 231 aa, synthetic construct
MGSSHHHHHHGSGNVEIIEKFAKEKIVAVLRANSVEEAIEKAVAVFAGGVGIIEITFTVPDADTVIKALSVLKEKGASIGAGTVTSVEQCRKAVEAGAEYIVSPHLDEEISQFCKELGIPYMPGVMTPTELVKAMKLGHKVLKLFPGEVVGPQFVKAMKKTYPDALFVPTGGVNLDNVAEWLKAGALAVGVGSALVKGTPDEVREKAKAFVEKVNASGVNAMKLVEKALGT

SEQ ID NO: 124 - protein, 231 aa, synthetic construct
MGSSHHHHHHGSGNVEIIEKFASEKIVAVLRANSVEEAIEKAVAVFAGGVGIIEITFTVPDADTVIKALSVLKEKGASIGAGTVTSVEQCRKAVEAGAEYIVSPHLDEEISQFCKELGIPYMPGVMTPTELVKAMKLGHKVLKLFPGEVVGPQFVKAMKKTYPDALFVPTGGVNLDNVAEWLKAGALAVGVGSALVKGTPDEVREKAKAFVEKVSASGVNSMELVKKALGT

SEQ ID NO: 125 - protein, 231 aa, synthetic construct
MGSSHHHHHHGSGNVEIIEKFASEKIVAVLRANSVEEAIEKAVAVFAGGVGIIEITFTVPDADTVIKALSVLKEKGASIGAGTVTSVEQCRKAVEAGAEYIVSPHLDEEISQFCKELGIPYMPGVMTPTELVKAMKLGHKVLKLFPGEVVGPQFVKAMKKTYPDALFVPTGGVNLDNVAEWLKAGALAVGVGSALVKGTPDEVREKAKAFVEKVSASGVNAMELVKKALGT

SEQ ID NO: 126 - protein, 232 aa, synthetic construct
MGSSHHHHHHGSGNPKEIIEKFASEKIVAVLRANSVEEAIEKAVAVFAGGVGIIEITFTVPDADTVIKALSVLKEKGASIGAGTVTSVEQCRKAVEAGAEYIVSPHLDEEISQFCKELGIPYMPGVMTPTELVKAMKLGHKVLKLFPGEVVGPQFVKAMKKTYPEALFVPTGGVNLDNVCEWFKAGALAVGVGSALVKGTPDEVREKAKAFVEKVSACGVNQMELVKKAEGT

SEQ ID NO: 127 - protein, 232 aa, synthetic construct
MGSSHHHHHHGSGNPKEIIEKFASEKIVAVLRANSVEEAIEKAVAVFAGGVGIIEITFTVPDADTVIKALSVLKEKGASIGAGTVTSVEQCRKAVEAGAEYIVSPHLDEEISQFCKELGIPYMPGVMTPTELVKAMKLGHKVLKLFPGEVVGPQFVKAMKKTYPEALFVPTGGVNLDNVCEWFKAGALAVGVGSALVKGTPDEVREKAKAFVEKVSACGVNSMELVKKAEGT

SEQ ID NO: 128 - protein, 233 aa, synthetic construct
MGSSHHHHHHGSGSGNKEIGEKFAEEKIVAVLRANSVEEAIEKAVAVFAGGVGIIEITFTVPDADTVIKALSVLKEKGASIGAGTVTSVEQCRKAVEAGAEYIVSPHLDEEISQFCKELGIPYMPGVMTPTELVKAMKLGHKVLKLFPGEVVGPQFVKAMKKTYPEALFVPTGGVNLDNVCEWLEAGALAVGVGSALVKGTPDEVREKAKAFVEKVNACPFNSMELFRKAEGT

SEQ ID NO: 129 - protein, 233 aa, synthetic construct
MGSSHHHHHHGSGSGNKEIGEKFATEKIVAVLRANSVEEAIEKAVAVFAGGVGIIEITFTVPDADTVIKALSVLKEKGASIGAGTVTSVEQCRKAVEAGAEYIVSPHLDEEISQFCKELGIPYMPGVMTPTELVKAMKLGHKVLKLFPGEVVGPQFVKAMKKTYPEALFVPTGGVNLDNVCEWLEAGALAVGVGSALVKGTPDEVREKAKAFVEKVRRCPFDSQELFKKAEGT

SEQ ID NO: 130 - protein, 233 aa, synthetic construct
MGSSHHHHHHGSGSGNKEIGEKFATEKIVAVLRANSVEEAIEKAVAVFAGGVGIIEITFTVPDADTVIKALSVLKEKGASIGAGTVTSVEQCRKAVEAGAEYIVSPHLDEEISQFCKELGIPYMPGVMTPTELVKAMKLGHKVLKLFPGEVVGPQFVKAMKKTYPEALFVPTGGVNLDNVCEWLEAGALAVGVGSALVKGTPDEVREKAKAFVEKVKACPFDSMELFKKAEGT

SEQ ID NO: 131 - protein, 233 aa, synthetic construct
MGSSHHHHHHGSGSGNKEIGEKFASEKIVAVLRANSVEEAIEKAVAVFAGGVGIIEITFTVPDADTVIKALSVLKEKGASIGAGTVTSVEQCRKAVEAGAEYIVSPHLDEEISQFCKELGIPYMPGVMTPTELVKAMKLGHKVLKLFPGEVVGPQFVKAMKKTYPEALFVPTGGVNLDNVCEWLEAGALAVGVGSALVKGTPDEVREKAKAFVEKVSACPFDSMELFKKAEGT

SEQ ID NO: 132 - protein, 233 aa, synthetic construct
MGSSHHHHHHGSGSGNEEMEELFASHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHKVLKLFPGEVVGPQFVKAMKGPFPEVLFVPTGGVNLDNVCEWLKAGVIAVGVGSALVKGTPDEVREKAKAFVEKVSACGVDSMAILSKVKGT

SEQ ID NO: 133 - protein, 233 aa, synthetic construct
MGSSHHHHHHGSGSGNEEMEELFAKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHKVLKLFPGEVVGPQFVKAMKGPFPEVLFVPTGGVNLDNVCEWLKAGVIAVGVGSALVKGTPDEVREKAKAFVEKVSACGVDSMAILEKVKGT

SEQ ID NO: 134 - protein, 233 aa, synthetic construct
MGSSHHHHHHGSGSGNEEMEELFASHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHKVLKLFPGEVVGPQFVKAMKGPFPEVLFVPTGGVNLDNVCEWFKAGVIAVGVGSALVKGTPDEVREKAKAFVEKISACGVDSMAILSKVKGT

SEQ ID NO: 135 - protein, 233 aa, synthetic construct
MGSSHHHHHHGSGSGNEEMEELFAKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHKVLKLFPGEVVGPQFVKAMKGPFPEVLFVPTGGVNLDNVCEWFKAGVIAVGVGSALVKGTPDEVREKAKAFVEKISACGVDSMAILEKVKGT

SEQ ID NO: 136 - protein, 238 aa, synthetic construct
MGSSHHHHHHGSGSEADLKMLKLFYQHRIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHKILKLFPGEVVGPQFVKAMKGPFPEVAFVPTGGVNLDNVCEWIEAGAVAVGVGSALVKGTPDEVREKAKAFVEKANLCAGKYKKQLDKQLALQLGT

SEQ ID NO: 137 - protein, 238 aa, synthetic construct
MGSSHHHHHHGSGSEADLKMLKLFYQHRIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHKILKLFPGEVVGPQFVKAMKGPFPEVAFVPTGGVNLDNVCEWFEAGVVAVGVGSALVKGTPDEVREKAKAFVEKINLCAGKYKKQLDKQLALQLGT

SEQ ID NO: 138 - protein, 242 aa, synthetic construct
MGSSHHHHHHGSGGHKILKLFPGEVVGPQFVKAMKGPFPDVAFVPTGGVNLDNVCEWFKAGVLAVGVGSALVKGTPDEVREKAKAFVEKINRCSEEAEKESEKIIKLETDPAALRMAKLFGKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGSGSGT

SEQ ID NO: 139 - protein, 238 aa, synthetic construct
MGSSHHHHHHGSGSEADLKMLKKFYTHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVPYMPGVMTPTELVKAMKLGHKLLKLFPGEVVGPQFVKAMKGPFPEVAFVPTGGVNLDNVCEWIEAGVVAVGVGSALVKGTPDEVREKAKAFVQKANECAGKYSDQLDKQLKLQLGT

SEQ ID NO: 140 - protein, 238 aa, synthetic construct
MGSSHHHHHHGSGSEADLKMLKKFYTHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVPYMPGVMTPTELVKAMKLGHKLLKLFPGEVVGPQFVKAMKGPFPEVAFVPTGGVNLDNVCEWIEAGVVAVGVGSALVKGTPDEVREKAKAFVQKANECAGKYSSQLDKQLSLQLGT

SEQ ID NO: 141 - protein, 239 aa, synthetic construct
MGSSHHHHHHGSTEAQALMLKLFVEHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHLVLKLFPGEVVGPQFVKAMKGPFPDVFFVPTGGVNLDNVCEWFKAGVLAVGVGSALVKGTPDEVREKAKAFVDKVTACIDAEVNAVHKKALAVAPKGT

SEQ ID NO: 142 - protein, 232 aa, synthetic construct
MGSSHHHHHHGSGNPKEMIELFASHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHKVLKLFPGEVVGPQFVKAMKGPFPEVLFVPTGGVNLDNVCEWFKAGVLAVGVGSALVKGTPDEVREKAKAFVEKISACGVNSMELVKKAEGT

SEQ ID NO: 143 - protein, 233 aa, synthetic construct
MGSSHHHHHHGSGSGNKEMGELFATHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHKILKLFPGEVVGPQFVKAMKGPFPEVLFVPTGGVNLDNVCEWLEAGVLAVGVGSALVKGTPDEVREKAKAFVEKVKACPFDSMELFKKAEGT

SEQ ID NO: 144 - protein, 233 aa, synthetic construct
MGSSHHHHHHGSGSGNKEMGELFASHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHKILKLFPGEVVGPQFVKAMKGPFPEVLFVPTGGVNLDNVCEWLEAGVLAVGVGSALVKGTPDEVREKAKAFVEKVSACPFDSMELFKKAEGT

SEQ ID NO: 145 - protein, 233 aa, synthetic construct
MGSSHHHHHHGSGSGNKEMGELFATHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHKILKLFPGEVVGPQFVKAMKGPFPEVLFVPTGGVNLDNVCEWFEAGVLAVGVGSALVKGTPDEVREKAKAFVEKIKACPFDSMELFKKAEGT

SEQ ID NO: 146 - protein, 233 aa, synthetic construct
MGSSHHHHHHGSGSGNKEMGELFASHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHKILKLFPGEVVGPQFVKAMKGPFPEVLFVPTGGVNLDNVCEWFEAGVLAVGVGSALVKGTPDEVREKAKAFVEKISACPFDSMELFKKAEGT

SEQ ID NO: 147 - protein, 238 aa, synthetic construct
MGSSHHHHHHGSGSEADLKMLKKLYQERIVAVLRANSVEEAIEKAVAVFAGGVKIIEITFTVPDADTVIKALSVLKEKGAIIGAGTVTSVEQCRKAVESGAEFIVSPHLDEEISQFCKEKGVPYMPGVMTPTELVKAMKLGHKLLKLFPGEVVGPQFVKAMKGPFPEVAFVPTGGVNLDNVCEWIEAGVVAVGVGSALVKGTPDEVREKAKAFVEKANLCAGKYKKQLDKQLALQLGT

SEQ ID NO: 148 - protein, 238 aa, synthetic construct
MGSSHHHHHHGSGSEADLKMLKKLYTEKIVAVLRANSVEEAIEKAVAVFAGGVKIIEITFTVPDADTVIKALSVLKEKGAIIGAGTVTSVEQCRKAVESGAEFIVSPHLDEEISQFCKEKGVPYMPGVMTPTELVKAMKLGHKLLKLFPGEVVGPQFVKAMKGPFPEVAFVPTGGVNLDNVCEWIEAGVVAVGVGSALVKGTPDEVREKAKAFVQKANECAGKYSDQLDKQLKLQLGT

SEQ ID NO: 149 - protein, 233 aa, synthetic construct
MGSSHHHHHHGSGSGNEEIEEKFASEKIVAVLRANSVEEAIEKAVAVFAGGVGIIEITFTVPDADTVIKALSVLKEKGASIGAGTVTSVEQCRKAVESGAEFIVSPHLDEEISQFCKEKGVPYMPGVMTPTELVKAMKLGHKVLKLFPGEVVGPQFVKAMKGPFPEVLFVPTGGVNLDNVCEWLKAGVIAVGVGSALVKGTPDEVREKAKAFVEKVSACGVDSMAILSKVKGT

SEQ ID NO: 150 - protein, 231 aa, synthetic construct
MGSSHHHHHHGSGNVEIIEKFAKEKIVAVLRANSVEEAIEKAVAVFAGGVGIIEITFTVPDADTVIKALSVLKEKGASIGAGTVTSVEQCRKAVESGAEFIVSPHLDEEISQFCKEKGVPYMPGVMTPTELVKAMKLGHKVLKLFPGEVVGPQFVKAMKGPFPDVLFVPTGGVNLDNVAEWLKAGVLAVGVGSALVKGTPDEVREKAKAFVEKVNASGVNAMKLVEKALGT

SEQ ID NO: 151 - protein, 231 aa, synthetic construct
MGSSHHHHHHGSGNVEIIEKFASEKIVAVLRANSVEEAIEKAVAVFAGGVGIIEITFTVPDADTVIKALSVLKEKGASIGAGTVTSVEQCRKAVESGAEFIVSPHLDEEISQFCKEKGVPYMPGVMTPTELVKAMKLGHKVLKLFPGEVVGPQFVKAMKGPFPDVLFVPTGGVNLDNVAEWLKAGVLAVGVGSALVKGTPDEVREKAKAFVEKVSASGVNSMELVKKALGT

SEQ ID NO: 152 - protein, 231 aa, synthetic construct
MGSSHHHHHHGSGNVEIIEKFASEKIVAVLRANSVEEAIEKAVAVFAGGVGIIEITFTVPDADTVIKALSVLKEKGASIGAGTVTSVEQCRKAVESGAEFIVSPHLDEEISQFCKEKGVPYMPGVMTPTELVKAMKLGHKVLKLFPGEVVGPQFVKAMKGPFPDVLFVPTGGVNLDNVAEWLKAGVLAVGVGSALVKGTPDEVREKAKAFVEKVSASGVNAMELVKKALGT

SEQ ID NO: 153 - protein, 232 aa, synthetic construct
MGSSHHHHHHGSGNPKEIIEKFASEKIVAVLRANSVEEAIEKAVAVFAGGVGIIEITFTVPDADTVIKALSVLKEKGASIGAGTVTSVEQCRKAVESGAEFIVSPHLDEEISQFCKEKGVPYMPGVMTPTELVKAMKLGHKVLKLFPGEVVGPQFVKAMKGPFPEVLFVPTGGVNLDNVCEWFKAGVLAVGVGSALVKGTPDEVREKAKAFVEKVSACGVNQMELVKKAEGT

SEQ ID NO: 154 - protein, 232 aa, synthetic construct
MGSSHHHHHHGSGNPKEIIEKFASEKIVAVLRANSVEEAIEKAVAVFAGGVGIIEITFTVPDADTVIKALSVLKEKGASIGAGTVTSVEQCRKAVESGAEFIVSPHLDEEISQFCKEKGVPYMPGVMTPTELVKAMKLGHKVLKLFPGEVVGPQFVKAMKGPFPEVLFVPTGGVNLDNVCEWFKAGVLAVGVGSALVKGTPDEVREKAKAFVEKVSACGVNSMELVKKAEGT

SEQ ID NO: 155 - protein, 233 aa, synthetic construct
MGSSHHHHHHGSGSGNKEIGEKFAKEKIVAVLRANSVEEAIEKAVAVFAGGVGIIEITFTVPDADTVIKALSVLKEKGASIGAGTVTSVEQCRKAVESGAEFIVSPHLDEEISQFCKEKGVPYMPGVMTPTELVKAMKLGHKVLKLFPGEVVGPQFVKAMKGPFPEVLFVPTGGVNLDNVCEWLEAGVLAVGVGSALVKGTPDEVREKAKAFVEKVNACPFNSMELFRKAEGT

SEQ ID NO: 156 - protein, 233 aa, synthetic construct
MGSSHHHHHHGSGSGNKEIGEKFATEKIVAVLRANSVEEAIEKAVAVFAGGVGIIEITFTVPDADTVIKALSVLKEKGASIGAGTVTSVEQCRKAVESGAEFIVSPHLDEEISQFCKEKGVPYMPGVMTPTELVKAMKLGHKVLKLFPGEVVGPQFVKAMKGPFPEVLFVPTGGVNLDNVCEWLEAGVLAVGVGSALVKGTPDEVREKAKAFVEKVRRCPFDSQELFKKAEGT

SEQ ID NO: 157 - protein, 233 aa, synthetic construct
MGSSHHHHHHGSGSGNKEIGEKFATEKIVAVLRANSVEEAIEKAVAVFAGGVGIIEITFTVPDADTVIKALSVLKEKGASIGAGTVTSVEQCRKAVESGAEFIVSPHLDEEISQFCKEKGVPYMPGVMTPTELVKAMKLGHKVLKLFPGEVVGPQFVKAMKGPFPEVLFVPTGGVNLDNVCEWLEAGVLAVGVGSALVKGTPDEVREKAKAFVEKVKACPFDSMELFKKAEGT

SEQ ID NO: 158 - protein, 233 aa, synthetic construct
MGSSHHHHHHGSGSGNKEIGEKFASEKIVAVLRANSVEEAIEKAVAVFAGGVGIIEITFTVPDADTVIKALSVLKEKGASIGAGTVTSVEQCRKAVESGAEFIVSPHLDEEISQFCKEKGVPYMPGVMTPTELVKAMKLGHKVLKLFPGEVVGPQFVKAMKGPFPEVLFVPTGGVNLDNVCEWLEAGVLAVGVGSALVKGTPDEVREKAKAFVEKVSACPFDSMELFKKAEGT

(19) United States
(12) Patent Application Publication
(10) Pub. No.: US 2025/0163400 A1
(43) Pub. Date: May 22, 2025

(54) NANOSTRUCTURE-FORMING POLYPEPTIDES AND USES THEREOF

(71) Applicant: Icosavax, Inc., Seattle, WA (US)

(72) Inventor: Quinton Dowling, Seattle, WA (US)

(21) Appl. No.: 18/954,071

(22) Filed: Nov. 20, 2024

Related U.S. Application Data
(60) Provisional application No. 63/601,517, filed on Nov. 21, 2023.
(60) Provisional application No. 63/552,288, filed on Feb. 12, 2024.

(51) Int. Cl. C12N 9/88; A61K 39/00; A61K 39/02; C07K 14/20

(52) U.S. Cl. CPC C12N 9/88; A61K 39/0225; C07K 14/20; C12Y 401/02014; A61K 2039/54; A61K 2039/544; A61K 2039/6031; A61K 2039/645; C07K 2319/735

(57) ABSTRACT

The present disclosure relates to polypeptides that are circular permutations of an I53-50A nanostructure, comprising, in N- to C-terminal order, a N-terminal polypeptide segment, a linking polypeptide segment, and a C-terminal polypeptide segment.

### CROSS REFERENCE TO RELATED APPLICATIONS

This application claims priority to U.S. provisional patent applications 63/601,517, filed on Nov. 21, 2023, and 63/552,288, filed on Feb. 12, 2024, the contents of each of which are incorporated herein by reference in their entireties.

### SEQUENCE LISTING

The instant application contains a Sequence Listing which has been submitted electronically in XML format and is hereby incorporated by reference in its entirety. Said XML copy created on Nov. 14, 2024, is named 061291-522001WO-new.xml and is 160 KB in size.

### TECHNICAL FIELD

The invention relates to protein nanostructures, computational methods used to design protein nanostructures, and uses thereof in, for example, vaccines.

### BACKGROUND

Protein nanostructures may be used to display vaccine antigens, as gene therapy vectors, or for other purposes.

The nanostructure I53-50, which is described in US 2016/0122392 A1, is one example of a computationally designed nanostructure. To design I53-50, the structures of the trimeric 2-keto-3-deoxy-6-phosphogluconate (KDPG) aldolase from Thermotoga maritima, (Protein Data Bank entry 1WA3) and the pentameric Lumazine synthase RibH2 from Mesorhizobium loti (20BX) were computationally docked to one another, such that the symmetry axes of the trimeric components and pentameric components align to the shared symmetry elements of an icosahedron; then the protein-protein interfaces between the trimeric components and the pentameric components were modified in silico to drive self-assembly of the two components into a nanostructure with icosahedral symmetry composed of trimers and pentamers having 3-fold and 5-fold symmetry axes-termed an 153 architecture. The resulting polypeptide sequences were expressed and purified, and it was shown experimentally that the polypeptides, as predicted, would self-assemble into the intended two-component nanostructure having 153 architecture. This two-component nanostructure (I53-50) comprised 60 copies of each polypeptide component, that is 20 copies of the designed trimeric component based on 1WA3 (termed “I53-50A”) and 12 copies of the designed pentameric component (termed “I53-50B”).

Another example of designed nanostructure, based on the same trimeric component and termed I3-01, is described in US 2018/0030429 A1. To design I3-01, the structure of KDPG (PDB entry 1WA3) was docked against itself alone; then the interfaces between the trimeric components were modified in silico to drive self-assembly of the designed nanostructure. The interface residues selected were different from the interface residues in the two-component nanostructure. The resulting single polypeptide sequence was expressed and purified, and it was shown experimentally that this polypeptide, as predicted would spontaneously self-assemble into a one-component icosahedral nanostructure having subunits aligned to the icosahedral 3-fold and new protein-protein interfaces on the icosahedral 2-fold symmetry axes-termed an 13 architecture. This one-component nanostructure (I3-01) comprised 60 copies of the polypeptide, which is 20 copies of the designed trimeric component.

Both of these designed nanostructures, I53-50 and I3-01, and variants thereof, have been employed to make vaccine candidates. For example, US 2020/0392187 A1 describes a two-component nanostructure composed of I53-50A fused the fusion (F) protein of a pneumovirus, and self-assembled with I53-50B to form an icosahedral nanostructure having a F protein trimer display on each three-fold axis. As another example, WO 2019/241483 A1 describes a one-component nanostructure composed of I3-01 C-terminally fused engineered envelope (Env) proteins of HIV-1. Nanostructures of this type have been shown to be effective vaccines and are currently in human clinical trials. Specifically, I53-50 nanostructures displaying F proteins from Respiratory Syncytial Virus (RSV) or human Metapneumovirus (hMPV), or the Spike (S) protein of SARS-CoV2, are in clinical trials as vaccines.

Nonetheless, there remains a need in the art for novel protein nanostructures capable of displaying other antigens. The present disclosure addresses that need.

### SUMMARY

The present disclosure provides a polypeptide that is a circular permutation of I53-50A, comprising an assembly domain, comprising, in N- to C-terminal order, a N-terminal polypeptide segment, a linking polypeptide segment, and a C-terminal polypeptide segment, wherein the N-terminal polypeptide segment comprises residues 74-201 of SEQ ID NO: 1 or a variant thereof, and the C-terminal polypeptide segment comprises residues 1-73 of SEQ ID NO: 1 or a variant thereof.

The present disclosure provides a polypeptide that is a circular permutation of I53-50A, comprising an assembly domain, comprising, in N- to C-terminal order, a N-terminal polypeptide segment, a linking polypeptide segment, and a C-terminal polypeptide segment, wherein the N-terminal polypeptide segment comprises residues 107-201 of SEQ ID NO: 1 or a variant thereof, and the C-terminal polypeptide segment comprises residues 1-106 of SEQ ID NO: 1 or a variant thereof.

The present disclosure provides a polypeptide that is a circular permutation of I53-50A, comprising an assembly domain, comprising, in N- to C-terminal order, a N-terminal polypeptide segment, a linking polypeptide segment, and a C-terminal polypeptide segment, wherein the N-terminal polypeptide segment comprises residues 128-201 of SEQ ID NO: 1 or a variant thereof, and the C-terminal polypeptide segment comprises residues 1-127 of SEQ ID NO: 1 or a variant thereof.

In some embodiments, variants of SEQ ID NO: 1 are at least 50%, at least 60%, at least 70%, at least 75%, at least 80%, at least 85%, at least 90%, at least 95%, at least 96%, at least 97%, at least 98%, or at least 99% identical to SEQ ID NO: 1.

In some embodiments, the N-terminal polypeptide segment and the C-terminal polypeptide segment comprises polypeptide sequences each selected from pairs A, B, or C provided in the Sequence Table, or from variants thereof having at least 50%, at least 60%, at least 70%, at least 75%, at least 80%, at least 85%, at least 90%, at least 95%, at least 96%, at least 97%, at least 98%, at least 99% identical thereto.

In some embodiments, the linking polypeptide segment comprises a polypeptide sequence at least 50%, at least 60%, at least 70%, at least 75%, at least 80%, at least 85%, at least 90%, at least 95%, at least 96%, at least 97%, at least 98%, at least 99%, or 100% identical to any of one of SEQ ID NOs: 8-21.

In some embodiments, the assembly domain comprises a polypeptide sequence at least 50%, at least 60%, at least 70%, at least 75%, at least 80%, at least 85%, at least 90%, at least 95%, at least 96%, at least 97%, at least 98%, at least 99%, or 100% identical to any one of SEQ ID NOs: 22-24.

In some embodiments, the assembly domain comprises a polypeptide sequence at least 50%, at least 60%, at least 70%, at least 75%, at least 80%, at least 85%, at least 90%, at least 95%, at least 96%, at least 97%, at least 98%, at least 99%, or 100% identical to a first polypeptide sequence in Table 2 or Table 4.

The present disclosure provides a polypeptide that is a variant of I53-50A having a C-terminal extension, comprising an assembly domain, the assembly domain comprising, in N- to C-terminal order, a base polypeptide segment and an extending polypeptide segment, wherein the base polypeptide segment comprises a polypeptide sequence at least 50%, at least 60%, at least 70%, at least 75%, at least 80%, at least 85%, at least 90%, at least 95%, at least 96%, at least 97%, at least 98%, at least 99% identical to residues 1-201 of SEQ ID NO: 1.

In some embodiments, the extending polypeptide segment comprises a polypeptide sequence at least 50%, at least 60%, at least 70%, at least 75%, at least 80%, at least 85%, at least 90%, at least 95%, at least 96%, at least 97%, at least 98%, at least 99%, or 100% identical to a polypeptide sequence in Table 1.

In some embodiments, the assembly domain comprises a polypeptide sequence at least 50%, at least 60%, at least 70%, at least 75%, at least 80%, at least 85%, at least 90%, at least 95%, at least 96%, at least 97%, at least 98%, at least 99%, or 100% identical to any one of SEQ ID NOs: 22-25.

In some embodiments, the assembly domain comprises a polypeptide sequence at least 50%, at least 60%, at least 70%, at least 75%, at least 80%, at least 85%, at least 90%, at least 95%, at least 96%, at least 97%, at least 98%, at least 99%, or 100% identical to a first polypeptide sequence in Table 2 or Table 4.

In some embodiments, the polypeptide comprises one or more amino acid residues at interface positions such that the polypeptide is capable of self-assemble to form a trimeric component of one-component nanostructure.

In some embodiments, the polypeptide comprises one or more amino acid residues at interface positions such that the polypeptide is capable of self-assemble to form a trimeric component of two-component nanostructure.

In some embodiments, the polypeptide self-assembles to form a trimeric component of a nanostructure, wherein the C terminus of the assembly domain is accessible on the surface of the nanostructure.

In some embodiments, the polypeptide self-assembles to form a trimeric component, optionally wherein the distance from the C terminus of the assembly domain to the three-fold axis is less than 30 Å, less than 25 Å, or less than 20 Å, or between 10 Å and 30 Å, between 15 Å and 30 Å, between 15 Å and 25 Å, or between 20 Å and 25 Å.

In some embodiments, the polypeptide self-assembles to form a soluble trimer, wherein the C terminus of the assembly domain is accessible on the surface of the soluble trimer.

In some embodiments, the polypeptide self-assembles to form a soluble trimer, wherein the C terminus of the assembly domain is proximal to the three-fold axis of the soluble trimer, optionally wherein the distance from the C terminus to the three-fold axis is less than 30 Å, less than 25 Å, or less than 20 Å, or between 10 Å and 30 Å, between 15 Å and 30 Å, between 15 Å and 25 Å, or between 20 Å and 25 Å.

In some embodiments, the polypeptide is a fusion protein comprising, in N- to C-terminal order, the assembly domain, optionally a polypeptide linker, and a heterologous polypeptide.

In some embodiments, the heterologous polypeptide is an antigen.

In some embodiments, the antigen is an ectodomain of a surface protein of a pathogenic organism, optionally a virus, or an antigenic fragment thereof.

In some embodiments, the antigen is an OspA or antigenic fragment thereof, preferably an OspA of Borrelia burgdorferi sensu lato.

In some embodiments, the antigen is an ectodomain of viral glycoprotein, or an antigenic fragment thereof.

In some embodiments, the antigen is an ectodomain of bacterial protein, or an antigenic fragment thereof.

The present disclosure provides a protein nanostructure, comprising a first component comprising a first polypeptide, and optionally a second component comprising a second polypeptide, wherein the first polypeptide is a polypeptide according to the present disclosure.

In some embodiments, the first component is a trimeric component comprising three copies of the first polypeptide.

In some embodiments, the nanostructure comprises the second component, and the second component comprises a second assembly domain comprising a polypeptide sequence at least 50%, at least 60%, at least 70%, at least 75%, at least 80%, at least 85%, at least 90%, at least 95%, at least 96%, at least 97%, at least 98%, at least 99%, or 100% identical to: SEQ ID NO: 26 or 27.

In some embodiments, the second component is a pentamer comprising five copies of the second polypeptide.

In some embodiments, the nanostructure comprises 20 copies of the first component.

In some embodiments, the nanostructure further comprises 12 copies of the second component.

In some embodiments, the C terminus of the first polypeptide is accessible on the surface of the nanostructure.

In some embodiments, the first polypeptide is a fusion protein comprising, in N- to C-terminal order, the first assembly domain, optionally a polypeptide linker, and a heterologous polypeptide.

In some embodiments, the heterologous polypeptide is an antigen.

In some embodiments, the antigen is an ectodomain of a surface protein of a pathogenic organism, optionally a virus, or an antigenic fragment thereof.

In some embodiments, the antigen is an OspA or antigenic fragment thereof, preferably an OspA of Borrelia burgdorferi sensu lato.

In some embodiments, the antigen is an ectodomain of viral glycoprotein, or an antigenic fragment thereof.

In some embodiments, the antigen is an ectodomain of bacterial protein, or an antigenic fragment thereof.

In some embodiments, the nanostructure has an 153 architecture and/or a quaternary structure substantially similar to I53-50.

The present disclosure provides a polynucleotide encoding a nanostructure disclosed herein or a polypeptide disclosed herein.

The present disclosure provides a delivery vehicle, comprising a polynucleotide disclosed herein, optionally a viral vector or a lipid nanoparticle.

The present disclosure provides a pharmaceutical composition, comprising a nanostructure disclosed herein, a polynucleotide disclosed herein, or a delivery vehicle disclosed herein, and a pharmaceutically acceptable carrier.

The present disclosure provides a vaccine, comprising a nanostructure disclosed herein, a polynucleotide disclosed herein, or a delivery vehicle disclosed herein, a pharmaceutically acceptable carrier, and optionally an adjuvant.

The present disclosure provides a host cell suitable for expression of a nanostructure disclosed herein or a polypeptide disclosed herein; and/or comprising a polynucleotide disclosed herein.

The present disclosure provides a method of making a polypeptide or nanostructure, comprising culturing a host cell disclosed herein under conditions suitable for expression of the polypeptide or nanostructure.

The present disclosure provides a method of generating an immune response to an antigen or to a pathogenic organism in a subject in need thereof, comprising administering to the subject a vaccine as disclosed herein, optionally via intramuscular injection or inhalation.

The present disclosure provides a method of immunizing a subject against infection by a pathogen, comprising administering to the subject a vaccine as disclosed herein, optionally via intramuscular injection or inhalation.

The present disclosure provides a composition or method as described herein.

Any aspect or embodiment described herein can be combined with any other aspect or embodiment as disclosed herein.

### BRIEF DESCRIPTION OF FIGURES

FIGS. 1A-1E show topology maps of CompA and circularly permuted versions. FIG. 1A: WT CompA topology. Alpha-helices are indicated by gray bars, labeled H1-H10. Beta-strands are indicated with white arrows, labeled E1-E8. FIG. 1B: Topology map of CompA with a de novo helical extension, H11, indicated with a dashed bar. FIG. 1C: Circular permutation of CompA with a cut-point between E3 and H4 (residues 73 and 74). FIG. 1D: Circular permutation of CompA with a cut-point between H5 and E5 (residues 106 and 107). FIG. 1E: Circular permutation of CompA with a cut-point between H6 and E6 (residues 127 and 128).

FIG. 2 shows small-scale immobilized metal affinity chromatography (IMAC) pull-down assay SDS-PAGE. IMAC pull-down flow through (FT), and elution (E) samples for select constructs, showing two bands of the expected size in the elution fraction.

FIG. 3 shows an example negative stain electron microscopy micrograph of a representative construct (CompA.024) with an OspA antigen genetically fused to the carboxy terminus demonstrating assembly into monodisperse VLPs of the expected size.

FIG. 4 shows an illustrative dynamic light scattering size distribution of a representative construct (CompA.024) with an OspA antigen genetically fused to the carboxy terminus demonstrating assembly into monodisperse VLPs of the expected size.

FIGS. 5A-5B show biolayer interferometry of antibody binding to an antigen fused to the carboxy-terminus of a representative construct (CompA.024), or to the amino-terminus of I53-50 CompA. Binding to LA-2 (FIG. 5A), which binds to an epitope on the carboxy-terminal end of the antigen, or binding to 221-7 (FIG. 5B), which binds to an epitope along the central domain of the antigen.

FIGS. 6A-C show structure models of representative designs. Extension of the carboxy terminus with parallel helical segments in grey (FIG. 6A). Extensions with an extended loop between the native carboxy terminus and the termini-extending helical segment (FIG. 6B). A circularly permuted design (FIG. 6C).

FIG. 7 shows representative particle size distributions for twenty-four constructs, designed to have improved assembly characteristics, assembled after IMAC purification. I53-50 is provided as a reference in each plot.

FIG. 8 shows the SEC chromatogram for four representative assemblies from purified components.

FIG. 9 shows particle size distributions of SEC representative purified VLPs compared to purified I53-50 VLP.

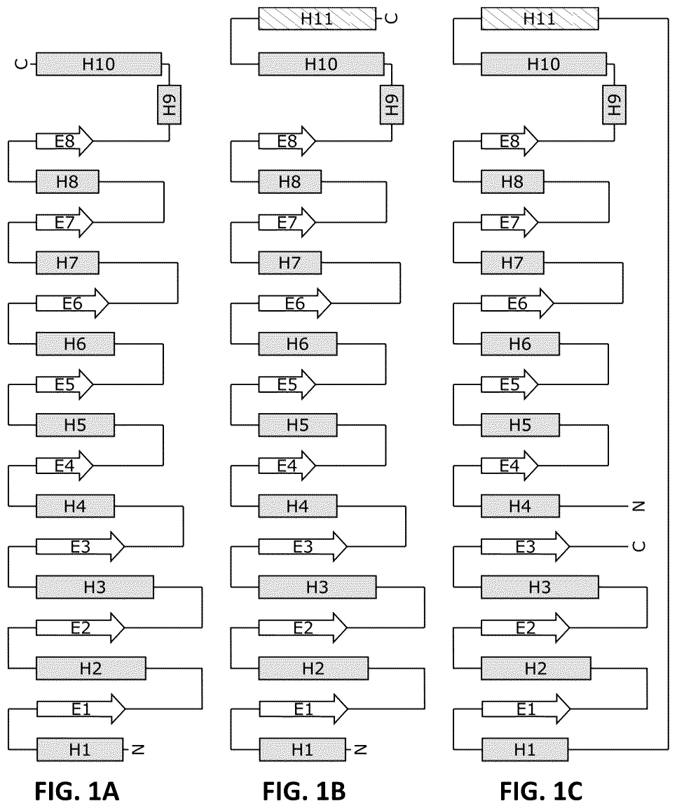

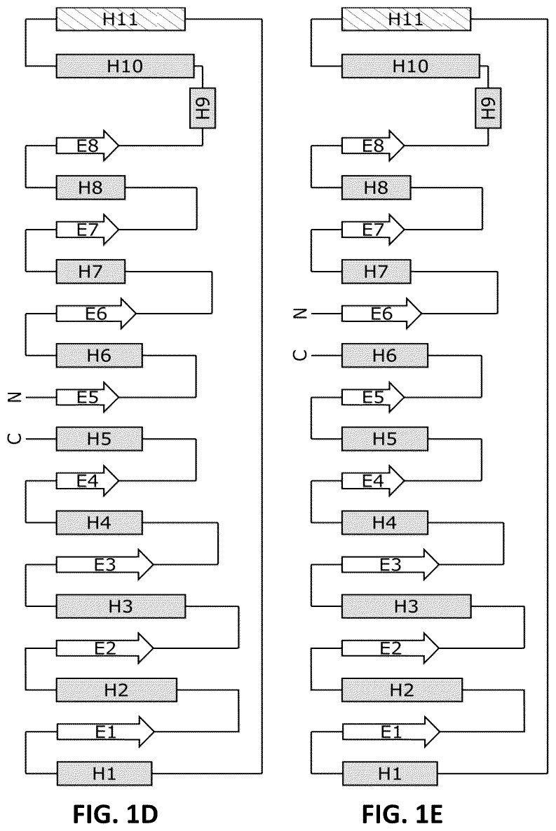

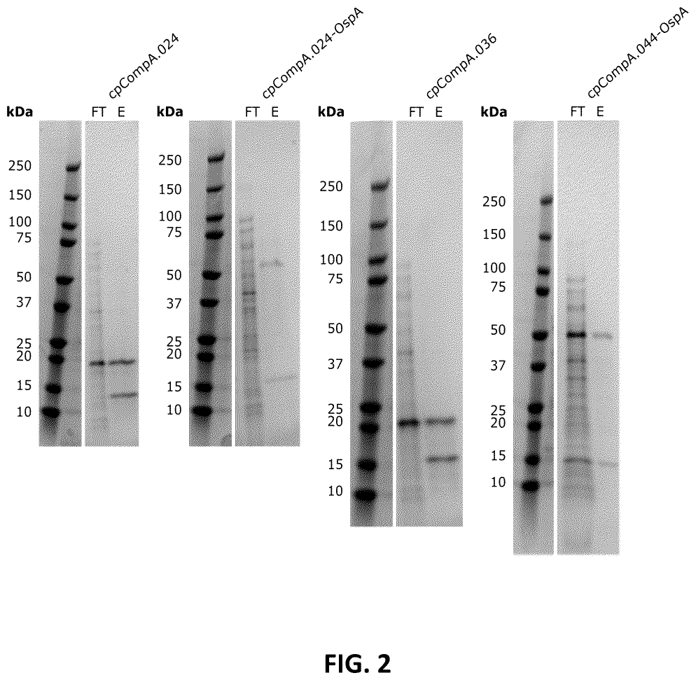

### DETAILED DESCRIPTION

The present disclosure relates generally to polypeptides for forming nanostructures, nanostructures, and uses thereof. In some embodiments, the disclosure provides polypeptides having disclosed sequences. In some embodiments, the polypeptides form nanostructure components in which the C terminus of the polypeptide is accessible on the surface of the nanostructure. In some embodiments, the polypeptide is a fusion protein comprising, in N- to C-terminal order, an assembly domain, optionally a linker, and a heterologous polypeptide, such as an antigen or antigenic fragment thereof.

Computationally designed protein nanomaterials are useful platforms for delivery of macromolecules, and vaccine design. The characteristics that make a particular nanomaterial useful include, but are not limited to, modularity, spontaneous self-assembly across a useful range of concentrations, stability, accessible termini, and particle size. Termini availability is constrained by the components used for designing a particular nanomaterial, and the orientation of the component within the designed architecture. Without wishing to be bound by theory, to ensure that any genetically linked domain is properly oriented with respect to the surface of the nanomaterial, the local structure of the termini is a contributing element. The present disclosure demonstrates that circular permutation can be an effective method for changing the accessibility of termini. In some embodiments, de novo designed termini extensions that are well ordered can also change termini accessibility. In some embodiments, both techniques are used to change the termini availability of a nanostructure (e.g., the protein nanomaterial I53-50). In some embodiments, a nanomaterial designed using circular permutation and/or de novo designed termini extensions may display the Borrelia burgdorferi sensu lato antigen OspA. In some embodiments, the techniques to change the termini availability described herein may be applied to a I3-01 protein nanomaterial.

The nanostructures of the present disclosure provide an antigen fused to the C terminus of a first component such that the antigen is displayed on the surface of the nanostructure. Without wishing to be bound by theory, fusion to the C terminus may increase or alter the immune response to the antigen. In some cases, fusion to the C terminus may promote induction of a protective and/or functional immune response in the subject. In embodiments, the nanostructures comprise a fusion between the C terminus and the N terminus of the first component via a novel linking polypeptide sequences as shown in Table 3. In embodiments, the nanostructures comprise sequence breaks which generate novel N- and C-termini as compared to a reference sequence fused to antigens or antigenic fragments.

The present disclosure provides a polypeptide that is a circular permutation of I53-50A, comprising an assembly domain, comprising, in N- to C-terminal order, a N-terminal polypeptide segment, a linking polypeptide segment, and a C-terminal polypeptide segment, wherein the N-terminal polypeptide segment comprises residues 74-201 of SEQ ID NO: 1 or a variant thereof, and the C-terminal polypeptide segment comprises residues 1-73 of SEQ ID NO: 1 or a variant thereof.

(I53-50A; SEQ ID NO: 1)
MEELFKKHKIVAVLRANSVEEAIEKAVAVFAGGVHLIEITFTVPDADTV
IKALSVLKEKGAIIGAGTVTSVEQCRKAVESGAEFIVSPHLDEEISQFC
KEKGVFYMPGVMTPTELVKAMKLGHTILKLFPGEVVGPQFVKAMKGPFP
NVKFVPTGGVNLDNVCEWFKAGVLAVGVGSALVKGTPDEVREKAKAFVE
KIRGCTE

The present disclosure provides a polypeptide that is a circular permutation of 153-50A, comprising an assembly domain, comprising, in N- to C-terminal order, a N-terminal polypeptide segment, a linking polypeptide segment, and a C-terminal polypeptide segment, wherein the N-terminal polypeptide segment comprises residues 107-201 of SEQ ID NO: 1 or a variant thereof, and the C-terminal polypeptide segment comprises residues 1-106 of SEQ ID NO: 1 or a variant thereof.

The present disclosure provides a polypeptide that is a circular permutation of I53-50A, comprising an assembly domain, comprising, in N- to C-terminal order, a N-terminal polypeptide segment, a linking polypeptide segment, and a C-terminal polypeptide segment, wherein the N-terminal polypeptide segment comprises residues 128-201 of SEQ ID NO: 1 or a variant thereof, and the C-terminal polypeptide segment comprises residues 1-127 of SEQ ID NO: 1 or a variant thereof.

In some embodiments, variants of SEQ ID NO: 1 are at least 50%, at least 60%, at least 70%, at least 75%, at least 80%, at least 85%, at least 90%, at least 95%, at least 96%, at least 97%, at least 98%, or at least 99% identical to SEQ ID NO: 1.

In some embodiments, the N-terminal polypeptide segment and the C-terminal polypeptide segment comprises polypeptide sequences each selected from pairs A, B, or C provided in the Sequence Table, or from variants thereof having at least 50%, at least 60%, at least 70%, at least 75%, at least 80%, at least 85%, at least 90%, at least 95%, at least 96%, at least 97%, at least 98%, at least 99% identical thereto.

In some embodiments, the N-terminal polypeptide segment and the C-terminal polypeptide segment comprises polypeptide sequences each selected from pairs A, B, or C, or from variants thereof at least 50%, at least 60%, at least 70%, at least 75%, at least 80%, at least 85%, at least 90%, at least 95%, at least 96%, at least 97%, at least 98%, at least 99% identical thereto:

| | N-terminal polypeptide segment | C-terminal polypeptide segment |
|---|---|---|
| A | MKMEELFKKHKIVAVLRANSVEE | EQCRKAVESGAEFIVSPHLDEEISQ |
| | AIEKAVAVFAGGVHLIEITFTVPD | FCKEKGVFYMPGVMTPTELVKA |
| | ADTVIKALSVLKEKGAIIGAGTVT | MKLGHDILKLFPGEVVGPQFVKA |
| | SV (SEQ ID NO: 2) | MKGPFPNVKFVPTGGVNLDNVCK |
| | | WFKAGVLAVGVGKALVKGKPDE |
| | | VREKAKKFVKKIR (SEQ ID NO: 5) |
| B | MKMEELFKKHKIVAVLRANSVEE | YMPGVMTPTELVKAMKLGHDILK |
| | AIEKAVAVFAGGVHLIEITFTVPD | LFPGEVVGPQFVKAMKGPFPNVK |
| | ADTVIKALSVLKEKGAIIGAGTVT | FVPTGGVNLDNVCKWFKAGVLA |
| | SVEQCRKAVESGAEFIVSPHLDEEI | VGVGKALVKGKPDEVREKAKKF |
| | SQFCKEKGVF (SEQ ID NO: 3) | VKKIR (SEQ ID NO: 6) |
| C | MKMEELFKKHKIVAVLRANSVEE | LKLFPGEVVGPQFVKAMKGPFPN |
| | AIEKAVAVFAGGVHLIEITFTVPD | VKFVPTGGVNLDNVCKWFKAGV |
| | ADTVIKALSVLKEKGAIIGAGTVT | LAVGVGKALVKGKPDEVREKAK |
| | SVEQCRKAVESGAEFIVSPHLDEEI | KFVKKIR (SEQ ID NO: 7) |
| | SQFCKEKGVFYMPGVMTPTELVK | |
| | AMKLGHDI (SEQ ID NO: 4) | |

In some embodiments, the linking polypeptide segment comprises a polypeptide sequence at least 50%, at least 60%, at least 70%, at least 75%, at least 80%, at least 85%, at least 90%, at least 95%, at least 96%, at least 97%, at least 98%, at least 99%, or 100% identical to any of one of SEQ ID NOs: 8-21.

In some embodiments, the assembly domain comprises a polypeptide sequence at least 50%, at least 60%, at least 70%, at least 75%, at least 80%, at least 85%, at least 90%, at least 95%, at least 96%, at least 97%, at least 98%, at least 99%, or 100% identical to any one of SEQ ID NOs: 22-24.

In some embodiments, the assembly domain comprises a polypeptide sequence at least 50%, at least 60%, at least 70%, at least 75%, at least 80%, at least 85%, at least 90%, at least 95%, at least 96%, at least 97%, at least 98%, at least 99%, or 100% identical to a first polypeptide sequence in Table 2 or Table 4.

The present disclosure provides a polypeptide that extends the C terminus of I53-50A, comprising an assembly domain, the assembly domain comprising, in N- to C-terminal order, a N-terminal polypeptide segment and an extending polypeptide segment, wherein the N-terminal polypeptide segment comprises a polypeptide sequence at least 50%, at least 60%, at least 70%, at least 75%, at least 80%, at least 85%, at least 90%, at least 95%, at least 96%, at least 97%, at least 98%, at least 99% identical to residues 1-201 of SEQ ID NO: 1.

In some embodiments, the extending polypeptide segment comprises a polypeptide sequence at least 50%, at least 60%, at least 70%, at least 75%, at least 80%, at least 85%, at least 90%, at least 95%, at least 96%, at least 97%, at least 98%, at least 99%, or 100% identical to a polypeptide sequence in Table 1.

As used herein, the term “linking polypeptide segment” refers to a polypeptide that connects the C terminus of a C-terminal polypeptide segment (e.g., a C-terminal polypeptide segment of I53-50A or a variant thereof) to an N-terminal polypeptide segment, to computationally generate a circular polypeptide chain in the process of circular permutation. After a circular polypeptide is computationally generated, breakpoints between the secondary structure elements are identified to create an N terminus for the designed polypeptide, which then may be expressed.

As used herein, the term “extending polypeptide segment” refers to a polypeptide that extends the C terminus of a base polypeptide segment (e.g., a polypeptide segment I53-50A or a variant thereof. In embodiments, the extending polypeptide segment may extend the C terminus to near to the N terminus of the base polypeptide segment without connecting the C terminus to the N terminus.

In some embodiments, the assembly domain comprises a polypeptide sequence at least 50%, at least 60%, at least 70%, at least 75%, at least 80%, at least 85%, at least 90%, at least 95%, at least 96%, at least 97%, at least 98%, at least 99%, or 100% identical to any one of SEQ ID NOs: 22-25.

In some embodiments, the assembly domain comprises a polypeptide sequence at least 50%, at least 60%, at least 70%, at least 75%, at least 80%, at least 85%, at least 90%, at least 95%, at least 96%, at least 97%, at least 98%, at least 99%, or 100% identical to a first polypeptide sequence in Table 2 or Table 4.

In some embodiments, the polypeptide comprises one or more amino acid residues at interface positions such that the polypeptide is capable of self-assemble to form a trimeric component of one-component nanostructure.

In some embodiments, the polypeptide comprises one or more amino acid residues at interface positions such that the polypeptide is capable of self-assemble to form a trimeric component of two-component nanostructure.

In some embodiments, the polypeptide self-assembles to form a trimeric component of a nanostructure, the C terminus of the assembly domain is accessible on the surface of the nanostructure.

In some embodiments, the polypeptide self-assembles to form a trimeric component, optionally wherein the distance from the C terminus of the assembly domain to the three-fold axis is less than 30 Å, less than 25 Å, or less than 20 Å, or between 10 Å and 30 Å, between 15 Å and 30 Å, between 15 Å and 25 Å, or between 20 Å and 25 Å.

In some embodiments, the polypeptide self-assembles to form a soluble trimer, the C terminus of the assembly domain is accessible on the surface of the soluble trimer.

In some embodiments, the polypeptide self-assembles to form a soluble trimer, the C terminus of the assembly domain is proximal to the three-fold axis of the soluble trimer, optionally wherein the distance from the C terminus to the three-fold axis is less than 30 Å, less than 25 Å, or less than 20 Å, or between 10 Å and 30 Å, between 15 Å and 30 Å, between 15 Å and 25 Å, or between 20 Å and 25 Å.

In some embodiments, the polypeptide is a fusion protein comprising, in N- to C-terminal order, the assembly domain, optionally a polypeptide linker, and a heterologous polypeptide.

In some embodiments, the heterologous polypeptide is an antigen.

In some embodiments, the antigen is an ectodomain of a surface protein of a pathogenic organism, optionally a virus, or an antigenic fragment thereof.

In some embodiments, the antigen is an OspA or antigenic fragment thereof, preferably an OspA of Borrelia burgdorferi sensu lato.

In some embodiments, the antigen is an ectodomain of viral glycoprotein, or an antigenic fragment thereof.

In some embodiments, the antigen is an ectodomain of bacterial protein, or an antigenic fragment thereof.

The present disclosure provides a protein nanostructure, comprising a first component comprising a first polypeptide, and optionally a second component comprising a second polypeptide, wherein the first polypeptide is a polypeptide according to the present disclosure.

In some embodiments, the first component is a trimeric component comprising three copies of the first polypeptide.

In some embodiments, the nanostructure comprises the second component, and the second component comprises a second assembly domain comprising a polypeptide sequence at least 50%, at least 60%, at least 70%, at least 75%, at least 80%, at least 85%, at least 90%, at least 95%, at least 96%, at least 97%, at least 98%, at least 99%, or 100% identical to: SEQ ID NO: 26 or 27.

In some embodiments, the second component is a pentamer comprising five copies of the second polypeptide.

In some embodiments, the nanostructure comprises 20 copies of the first component.

In some embodiments, the nanostructure further comprises 12 copies of the second component.

In some embodiments, the C terminus of the first polypeptide is accessible on the surface of the nanostructure.

In some embodiments, the first polypeptide is a fusion protein comprising, in N- to C-terminal order, the first assembly domain, optionally a polypeptide linker, and a heterologous polypeptide.

In some embodiments, the heterologous polypeptide is an antigen.

In some embodiments, the antigen is an ectodomain of a surface protein of a pathogenic organism, optionally a virus, or an antigenic fragment thereof.

In some embodiments, the antigen is an OspA or antigenic fragment thereof, preferably an OspA of Borrelia burgdorferi sensu lato.

In some embodiments, the antigen is an ectodomain of viral glycoprotein, or an antigenic fragment thereof.

In some embodiments, the antigen is an ectodomain of bacterial protein, or an antigenic fragment thereof.

In some embodiments, the nanostructure has an 153 architecture and/or a quaternary structure substantially similar to I53-50.

The present disclosure provides a polynucleotide encoding a nanostructure disclosed herein or a polypeptide disclosed herein.

The present disclosure provides a delivery vehicle, comprising a polynucleotide disclosed herein, optionally a viral vector or a lipid nanoparticle.

The present disclosure provides a pharmaceutical composition, comprising a nanostructure disclosed herein, a polynucleotide disclosed herein, or a delivery vehicle disclosed herein, and a pharmaceutically acceptable carrier.

The present disclosure provides a vaccine, comprising a nanostructure disclosed herein, a polynucleotide disclosed herein, or a delivery vehicle disclosed herein, a pharmaceutically acceptable carrier, and optionally an adjuvant.

The present disclosure provides a host cell suitable for expression of a nanostructure disclosed herein or a polypeptide disclosed herein; and/or comprising a polynucleotide disclosed herein.

The present disclosure provides a method of making a polypeptide or nanostructure, comprising culturing a host cell disclosed herein under conditions suitable for expression of the polypeptide or nanostructure.

The present disclosure provides a method of generating an immune response to an antigen or to a pathogenic organism in a subject in need thereof, comprising administering to the subject a vaccine as disclosed herein, optionally via intramuscular injection or inhalation.

The present disclosure provides a method of immunizing a subject against infection by a pathogen, comprising administering to the subject a vaccine as disclosed herein, optionally via intramuscular injection or inhalation.

The present disclosure provides a composition or method as described herein.

### Polypeptides

Patent Pub No. US 2015/0356240 A1 describes various methods for designing protein assemblies. As described in US Patent Pub No. US 2016/0122392 A1 and in International Patent Pub. No. WO 2014/124301 A1, isolated polypeptides were designed for their ability to self-assemble in pairs to form protein nanostructures, such as icosahedral particles. The design involved design of suitable interface residues for each member of the polypeptide pair that can be assembled to form the protein nanostructures. The protein nanostructures so formed include symmetrically repeated, non-natural, non-covalent polypeptide-polypeptide interfaces that orient a first assembly domain and a second assembly domain into protein nanostructures, such as one with an icosahedral symmetry.

Thus, in one embodiment a first assembly domain and second assembly domain of the component are selected from the Sequence Table. In each case, an N-terminal methionine residue present in the full-length protein is included, but may be removed to make a fusion that is not included in the sequence. The identified residues in the Sequence Table are numbered beginning with an N-terminal methionine. In various embodiments, one or more additional residues are deleted from the N terminus and/or additional residues are added to the N terminus. In some embodiments, the interface residues of I53-50A (SEQ ID NO: 1) first assembly domain are 25, 29, 33, 54, and 57. In some embodiments, the interface residues of I53-50B (SEQ ID NO: 27) or I53-50B.4PosT1 (SEQ ID NO: 26) second assembly domain are 24, 28, 36, 124, 125, 127, 128, 129, 131, 132, 133, 135, and 139.

The pair of sequences together form an 153 multimer with icosahedral symmetry. The interface residues identified are residue numbers in each illustrative polypeptide that were identified as present at the interface of resulting assembled protein nanostructures (i.e., “identified interface residues”). As can be seen, the number of interface residues for the illustrative polypeptides of SEQ ID NOs: 1 and (26 or 27) range from 4-13. In various embodiments, a first assembly domain and second assembly domain comprise an amino acid sequence that is at least 70%, 75%, 80%, 85%, 90%, 91%, 92%, 93%, 94%, 95%, 96%, 97%, 98%, 99%, or 100% identical over its length, and identical at least at 1, 2, 3, 4, 5, 6, 7, 8, 9, 10, 11, 12, or 13 identified interface positions (depending on the number of interface residues for a given polypeptide), to the amino acid sequence of a polypeptide selected from the group consisting of SEQ ID NOs: 1 and (26 or 27).

In some embodiments, a polypeptide for forming a nanostructure comprises a first assembly domain. In some embodiments, a polypeptide for forming a nanostructure comprises a first assembly domain comprising a polypeptide sequence at least 50%, at least 60%, at least 70%, at least 75%, at least 80%, at least 85%, at least 90%, at least 95%, at least 96%, at least 97%, at least 98%, at least 99%, or 100% identical to a first polypeptide sequence in Table 2 or Table 4.

In some embodiments, a protein nanostructure comprises a first component and optionally, a second component. In some embodiments, the first component comprises a first polypeptide comprising a first assembly domain. In some embodiments, a protein nanostructure, comprises a first component, and optionally a second component, wherein the first component comprises a first polypeptide comprising a first assembly domain comprising a polypeptide sequence at least 50%, at least 60%, at least 70%, at least 75%, at least 80%, at least 85%, at least 90%, at least 95%, at least 96%, at least 97%, at least 98%, at least 99%, or 100% identical to a polypeptide sequence in Table 2 or Table 4.

In some embodiments, the first component is a trimeric component comprising three copies of the first polypeptide.

In some embodiments, the nanostructure comprises the second component. In some embodiments, the second component comprises a second polypeptide comprising a second assembly domain. In some embodiments, the nanostructure comprises the second component comprising a second polypeptide comprising a second assembly domain comprising a polypeptide sequence at least 50%, at least 60%, at least 70%, at least 75%, at least 80%, at least 85%, at least 90% at least 95% at least 96% at least 97% at least 98% at least 99% or 100% identical to:

(I53-50B.4PosT1; SEQ ID NO: 26)
NQHSHKDHETVRIAVVRARWHAEIVDACVSAFEAAMRDIGGDRFAVDVF
DVPGAYEIPLHARTLAETGRYGAVLGTAFVVNGGIYRHEFVASAVINGM
MNVQLNTGVPVLSAVLTPHNYDKSKAHTLLFLALFAVKGMEAARACVEI
LAAREKIAA;
or
(I53-50B; SEQ ID NO: 27)
NQHSHKDYETVRIAVVRARWHAEIVDACVSAFEAAMADIGGDRFAVDVF
DVPGAYEIPLHARTLAETGRYGAVLGTAFVVNGGIYRHEFVASAVIDGM
MNVQLSTGVPVLSAVLTPHRYRDSDAHTLLFLALFAVKGMEAARACVEI
LAAREKIAA.

In some embodiments, the second component is a pentamer comprising five copies of the second polypeptide.

In some embodiments, the first component comprises a first polypeptide comprising a first assembly domain comprising a polypeptide sequence at least 50%, at least 60%, at least 70%, at least 75%, at least 80%, at least 85%, at least 90%, at least 95%, at least 96%, at least 97%, at least 98%, at least 99%, or 100% identical to a polypeptide sequence in Table 2 or Table 4 and the second component comprising a second polypeptide comprising a second assembly domain comprising a polypeptide sequence at least 50%, at least 60%, at least 70%, at least 75%, at least 80%, at least 85%, at least 90%, at least 95%, at least 96%, at least 97%, at least 98%, at least 99%, or 100% identical to:

(I53-50B.4PosT1; SEQ ID NO: 26)
NQHSHKDHETVRIAVVRARWHAEIVDACVSAFEAAMRDIGGDRFAVDVF
DVPGAYEIPLHARTLAETGRYGAVLGTAFVVNGGIYRHEFVASAVINGM
MNVQLNTGVPVLSAVLTPHNYDKSKAHTLLFLALFAVKGMEAARACVEI
LAAREKIAA;
or
(I53-50B; SEQ ID NO: 27)
NQHSHKDYETVRIAVVRARWHAEIVDACVSAFEAAMADIGGDRFAVDVF
DVPGAYEIPLHARTLAETGRYGAVLGTAFVVNGGIYRHEFVASAVIDGM
MNVQLSTGVPVLSAVLTPHRYRDSDAHTLLFLALFAVKGMEAARACVEI
LAAREKIAA.

In some embodiments, the nanostructure comprises 20 copies of the first component.

In some embodiments, the nanostructure further comprises 12 copies of the second component.

In some embodiments, the C terminus of the first polypeptide is accessible on the surface of the nanostructure.

In some embodiments, the first polypeptide comprises one or more amino acid residues at interface positions such that the polypeptide is capable of self-assembling to form a trimeric component of a one-component nanostructure.

In some embodiments, the first polypeptide comprises one or more amino acid residues at interface positions such that the polypeptide is capable of self-assembling to form a trimeric component of a two-component nanostructure.

In some embodiments, when the first polypeptide self-assembles to form a trimeric component of a nanostructure, the C terminus of the assembly domain is accessible on the surface of the nanostructure.

In some embodiments, when the first polypeptide self-assembles to form a trimeric component, the C terminus of the assembly domain is proximal to the three-fold axis of the trimeric component. In some embodiments, the distance from the C terminus to the three-fold axis is less than 30 Å, less than 25 Å, or less than 20 Å, or between 10 Å and 30 Å, between 15 Å and 30 Å, between 15 Å and 25 Å, or between 20 Å and 25 Å.

### Antigens

In some embodiments, a protein nanostructure comprises a first component and optionally, a second component. In such embodiment, the first component comprises a first polypeptide comprising a first assembly domain and optionally, a second component comprising a second polypeptide comprising a second assembly domain.

In some embodiments, the first polypeptide is a fusion protein comprising, in N- to C-terminal order, the first assembly domain, optionally a linker, and a heterologous polypeptide sequence, preferably an antigen.

In some embodiments, the antigen is an ectodomain of a surface protein of a pathogenic organism, or an antigenic fragment thereof. In some embodiments, the antigen is an ectodomain of a surface protein of a virus, or an antigenic fragment thereof.

Without wishing to be bound by theory, antigens may be natively anchored at or near their N terminus.

Antigens that have N-terminal attachments in their native orientations may exhibit improved stability or antigenicity when fused to the C terminus of a first assembly domain as compared to an antigen fused to the N terminus of a first assembly domain.

In some embodiments, the antigen is fused to the C terminus of the first assembly domain.

Illustrative bacterial surface proteins that are natively anchored at their N-termini include, but are not limited to, OspA of Borrelia burgdorferi sensu lato, OspB of Borrelia burgdorferi sensu lato, fHbp of N. Meningitidis, bacterial type II membrane proteins that are asymmetric or comprise a C3-symmetric oligomer, and bacterial proteins with lipidation sites towards the N-terminal side of the protein that are asymmetric or comprise a C3-symmetric oligomer. Illustrative non-bacterial surface proteins that are natively anchored at their N-termini include, but are not limited to, paramyxovirus and/or pneumovirus G proteins.

In some embodiments, the antigen is an OspA or antigenic fragment thereof. In some embodiments, the antigen is an OspA of Borrelia burgdorferi sensu lato (SEQ ID NO: 28). In some embodiments, the antigen is an OspB or antigenic fragment thereof. In some embodiments, the antigen is an OspB of Borrelia burgdorferi sensu lato (SEQ ID NO: 29).

In some embodiments, the antigen is fHbp of N. Meningitidis (SEQ ID NO: 30) or an antigenic fragment thereof.

In some embodiments, the antigen is an antigen derived from a bacterial pathogen that exhibits asymmetric type II membrane geometry.

In some embodiments, the antigen is an antigen derived from a bacterial pathogen that exhibits C3-symmetric oligomer geometry.

In some embodiments, the antigen is an antigen derived from a bacterial pathogen that comprises lipidation sites on the N-terminal portion of the protein and exhibits asymmetric type II membrane geometry.

In some embodiments, the antigen is an antigen derived from a bacterial pathogen that comprises lipidation sites on the N-terminal portion of the protein and exhibits C3-symmetric oligomer geometry.

In some embodiments, the antigen is an RSV G protein (SEQ ID NO: 31) or an antigenic fragment thereof. In some embodiments, the antigen is an hMPV G protein (SEQ ID NO: 32) or an antigenic fragment thereof.

In some embodiments, the antigen is a paramyxovirus and/or pneumovirus G protein or an antigenic fragment thereof.

In some embodiments, the antigen is a S1 C-terminal domains of coronavirus, a RBD of paramyxovirus G, H or HN proteins (e.g., Nipah/Hendra G, PIV3 HN, Measles H, Mumps HN), an HA head domain of influenza, a fusion domain of a class III fusion protein (e.g., CMV, EBV, HSV, VZV, Rabies), gp120 of HIV Env, an engineered antigen (e.g., eOD), rotavirus VP8 domain, a segment/domain of P. Falciparum CSP, a segment/domain of B. Burgdorferi sensu lato OspA (e.g., the C-terminal domain).

In some embodiments, the antigen is an ectodomain of a viral glycoprotein, or an antigenic fragment thereof.

In some embodiments, the antigen is an ectodomain of a parasitic protein, or an antigenic fragment thereof. In some embodiments, the parasitic protein is from a plasmodium parasite.

In some embodiments, the antigen is an ectodomain of bacterial protein, or an antigenic fragment thereof.

### Polynucleotides, Vectors, and Host Cells

The present disclosure provides a polynucleotide encoding a nanostructure of any of the embodiments herein or a polypeptide of any of the embodiments herein.

The present disclosure provides a vector comprising a polynucleotide encoding a nanostructure of any of the embodiments herein or a polypeptide of any of the embodiments herein.

The present disclosure provides a host cell suitable for expression of a nanostructure of any of the embodiments herein or a polypeptide of any of the embodiments herein; and/or comprising a polynucleotide of any of the embodiments herein.

The present disclosure provides a method of making a polypeptide or nanostructure, comprising culturing a host cell of any of the embodiments herein under conditions suitable for expression of a polypeptide or nanostructure of any of the embodiments herein.

In another aspect, the disclosure provides a polynucleotide encoding any of the foregoing polypeptides. The polynucleotide may be an mRNA, such as a modified mRNA. The disclosure further provides vectors that include any of these polynucleotides. The vector may be a viral vector, such as an adenovirus vector, or a non-viral vector, such as a lipid nanoparticle (LNP). The disclosure further provides host cells that are transfected or transformed with any of the foregoing polynucleotides.

In an aspect, the disclosure provides a method of making a protein nanostructure involving culturing a host cell under conditions suitable to cause the expression of one or more components of a nanostructure, alone or separately; purifying the components, alone or separately; contacting solutions of the purified components; and/or incubating the components under condition suitable for self-assembly of the components to form a nanostructure.

### Pharmaceutical Compositions and Vaccines

The disclosure also provides pharmaceutical compositions. Such pharmaceutical compositions can be used for generating an immune response against an infectious disease in a subject. The pharmaceutical compositions of the disclosure may include a pharmaceutically acceptable carrier. A thorough discussion of such carriers is available in Chapter 30 of Remington: The Science and Practice of Pharmacy (23^{rd} ed., 2021).

In some embodiments, the pharmaceutical composition can also include excipients and/or additives. Examples of these are surfactants, stabilizers, complexing agents, antioxidants, or preservatives which prolong the duration of use of the finished pharmaceutical formulation, flavorings, vitamins, or other additives known in the art. Complexing agents include, but are not limited to, ethylenediaminetetraacetic acid (EDTA) or a salt thereof, such as the disodium salt, citric acid, nitrilotriacetic acid and the salts thereof. In some embodiments, preservatives include, but are not limited to, those that protect the solution from contamination with pathogenic particles, including benzalkonium chloride or benzoic acid, or benzoates such as sodium benzoate. Antioxidants include, but are not limited to, vitamins, provitamins, ascorbic acid, vitamin E, salts or esters thereof.

Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethyl cellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

In some embodiments, one or more tonicity agents may be added to provide the desired ionic strength. Tonicity agents for use herein include those which display no or only negligible pharmacological activity after administration. Both inorganic and organic tonicity adjusting agents may be used.

In another aspect, the disclosure provides a pharmaceutical composition comprising a polypeptide, a protein complex, or a nanostructure of the disclosure.

The present disclosure provides a pharmaceutical composition comprising a nanostructure of any of the embodiments herein.

The present disclosure provides a vaccine comprising a nanostructure of any of the embodiments herein.

In another aspect, the disclosure provides pharmaceutical composition or vaccines. In embodiments, pharmaceutical composition includes a nanostructure as described herein in therapeutically effective amount. In embodiments, the vaccine includes a nanostructure in an amount effective to generate an immune response in a subject. Nanostructures used in vaccines may be complexed with, conjugated to, or fused to an antigen. The antigen may be a polypeptide derived from a pathogenic organism, or an antigenic fragment thereof.

In other embodiments, the pharmaceutical composition or vaccines includes a polynucleotide encoding a nanostructure as disclosed herein, such as an mRNA, or vector as disclosed herein, such an LNP—in each case in a therapeutically effective amount or in an amount effective to generate an immune response.

In another aspect, the disclosure provides methods of treating and/or preventing a disease or disorder in a subject in need thereof, as well as methods of generating an immune response to a pathogenic organism in a subject. Such methods may comprise administering a nanostructure, pharmaceutical composition, or vaccine according to the disclosure to the subject by intramuscular, intravenous, or intranasal.

Further provided are kits and pre-filled syringes that include any of the foregoing compositions.

### Vaccines

In another aspect, the disclosure provides a vaccine comprising a polypeptide, a protein complex, or a nanostructure as disclosed herein.

In some embodiments, the vaccine comprises an adjuvant.

### Adjuvants

Adjuvants or immune potentiators may also be administered with or in combination with lipid nanoparticle composition. Advantages of adjuvants include, but are not limited to, the enhancement of the immunogenicity of antigens, modification of the nature of the immune response, the reduction of the antigen amount needed for a successful immunization, the reduction of the frequency of booster immunizations needed and an improved immune response in elderly and immunocompromised vaccinees. These may be co-administered by any route, e.g., intramuscular, subcutaneous, intravenous, or intradermal injections.

Adjuvants may include, but are not limited to, a natural or a synthetic adjuvant. Adjuvants may be organic or inorganic.

Adjuvants may be selected from any of the classes (1) mineral salts, e.g., aluminum hydroxide and aluminum or calcium phosphate gels; (2) emulsions including: oil emulsions and surfactant based formulations, e.g., microfluidised detergent stabilized oil-in-water emulsion, purified saponin, oil-in-water emulsion, stabilized water-in-oil emulsion; (3) particulate adjuvants, e.g., virosomes (unilamellar liposomal vehicles incorporating influenza hemagglutinin), structured complex of saponins and lipids, polylactide co-glycolide (PLG); (4) microbial derivatives; (5) endogenous human immunomodulators; (6) inert vehicles, such as gold particles; (7) microorganism derived adjuvants; (8) tensoactive compounds; (9) carbohydrates; or combinations thereof.

Adjuvants for nucleic acid vaccines (DNA) have been disclosed in, for example, Kobiyama, et al., Vaccines, 2013, 1(3), 278-292, the contents of which are incorporated herein by reference in their entirety. Any of the adjuvants disclosed by Kobiyama et al., may be used in the vaccines as described herein.

Other adjuvants which may be utilized include any of those listed on the web-based vaccine adjuvant database, on the World Wide Web at violinet.org/vaxjo/and described in for example Sayers, et al., J. Biomedicine and Biotechnology, volume 2012 (2012), Article ID 831486, 13 pages, the contents of which are incorporated herein by reference in their entirety.

Specific adjuvants may include cationic liposome-DNA complex JVRS-100, aluminum hydroxide vaccine adjuvant, aluminum phosphate vaccine adjuvant, aluminum potassium sulfate adjuvant, alhydrogel, ISCOM(s)™, Freund's Complete Adjuvant, Freund's Incomplete Adjuvant, CpG DNA Vaccine Adjuvant, Cholera toxin, Cholera toxin B subunit, Liposomes, Saponin Vaccine Adjuvant, DDA Adjuvant, Squalene-based Adjuvants, Etx B subunit Adjuvant, IL-12 Vaccine Adjuvant, LTK63 Vaccine Mutant Adjuvant, TiterMax Gold Adjuvant, Ribi Vaccine Adjuvant, Montanide ISA 720 Adjuvant, Corynebacterium-derived P40 Vaccine Adjuvant, MPL™ Adjuvant, AS04, AS02, AS01_{E}, Lipopolysaccharide Vaccine Adjuvant, Muramyl Dipeptide Adjuvant, CRL1005, Killed Corynebacterium parvum Vaccine Adjuvant, Montanide ISA 51, Bordetella pertussis component Vaccine Adjuvant, Cationic Liposomal Vaccine Adjuvant, Adamantylamide Dipeptide Vaccine Adjuvant, Arlacel A, VSA-3 Adjuvant, Aluminum vaccine adjuvant, Polygen Vaccine Adjuvant, ADJUMER™, Algal Glucan, Bay R1005, Theramide®, Stearyl Tyrosine, Specol, Algammulin, AVRIDINE®, Calcium Phosphate Gel, CTA1-DD gene fusion protein, DOC/Alum Complex, Gamma Inulin, Gerbu Adjuvant, GM-CSF, GMDP, Recombinant hIFN-gamma/Interferon-g, Interleukin-10, Interleukin-2, Interleukin-7, Sclavo peptide, Rehydragel LV, Rehydragel HPA, Loxoribine, MF59, MTP-PE Liposomes, Murametide, Murapalmitine, D-Murapalmitine, NAGO, Non-Ionic Surfactant Vesicles, PMMA, Protein Cochleates, QS-21, SPT (Antigen Formulation), nanoemulsion vaccine adjuvant, AS03, Quil-A vaccine adjuvant, RC529 vaccine adjuvant, LTR192G Vaccine Adjuvant, E. coli heat-labile toxin, LT, amorphous aluminum hydroxyphosphate sulfate adjuvant, Calcium phosphate vaccine adjuvant, Montanide Incomplete Seppic Adjuvant, Imiquimod, Resiquimod, AF03, Flagellin, Poly(I:C), ISCOMATRIX®, Abisco-100 vaccine adjuvant, Albumin-heparin microparticles vaccine adjuvant, AS-2 vaccine adjuvant, B7-2 vaccine adjuvant, DHEA vaccine adjuvant, Immunoliposomes Containing Antibodies to Costimulatory Molecules, SAF-1, Sendai Proteoliposomes, Sendai-containing Lipid Matrices, Threonyl muramyl dipeptide (TMDP), Ty Particles vaccine adjuvant, Bupivacaine vaccine adjuvant, DL-PGL (Polyester poly (DL-lactide-co-glycolide)) vaccine adjuvant, IL-15 vaccine adjuvant, LTK72 vaccine adjuvant, MPL-SE vaccine adjuvant, non-toxic mutant E112K of Cholera Toxin mCT-E112K, and/or Matrix-S.

In some embodiments, the adjuvant comprises squalene. In some embodiments, the adjuvant comprises aluminum hydroxide. In some embodiments, the adjuvant comprises AS01_{E}.

### Methods of Use

The present disclosure provides a method of making a polypeptide or nanostructure, comprising culturing a host cell disclosed herein under conditions suitable for expression of the polypeptide or nanostructure.

The present disclosure provides a method of generating an immune response to an antigen or to a pathogenic organism in a subject in need thereof, comprising administering to the subject a nanostructure of the present disclosure.

The present disclosure provides a method of generating an immune response to an antigen or to a pathogenic organism in a subject in need thereof, comprising administering to the subject a vaccine as disclosed herein, optionally via intramuscular injection or inhalation.

The present disclosure provides a method of generating high titers of functional antibodies against an antigen or to a pathogenic organism.

The present disclosure provides a method of immunizing a subject against infection by a pathogen, comprising administering to the subject a vaccine as disclosed herein, optionally via intramuscular injection or inhalation.

In another aspect, the disclosure provides methods of administration for the composition, the pharmaceutical composition, or the vaccine described herein.

In another aspect, the disclosure provides a method of vaccinating a subject, comprising administering to the subject a composition described herein. In another aspect, the disclosure provides a method of generating an immune response in a subject, comprising administering to the subject a composition described herein. In another aspect, the disclosure provides a method of treating or preventing disease in a subject, comprising administering to the subject a composition described herein. In another aspect, the disclosure provides a composition of the disclosure for use in vaccinating, generating an immune response, or treating or preventing disease. In another aspect, the disclosure provides a composition, method, or use as described herein. In another aspect, the disclosure provides a method of making a composition, comprising culturing host cells modified to express one or more polypeptides as described herein.

In some embodiments, the method comprising administering the vaccine described herein. In some embodiments, the vaccine is administered by subcutaneous injection. In some embodiments, the vaccine is administered by intramuscular injection. In some embodiments, the vaccine is administered by intradermal injection. In some embodiments, the vaccine is administered intranasally. In one aspect, the disclosure provides a pre-filled syringe comprising the vaccine described herein. In one aspect, the disclosure provides a kit comprising the vaccine described herein or the pre-filled syringe described herein.

In some embodiments, the unit dose of the pharmaceutical composition comprises about 0.5 μg to about 1 μg, about 20 μg to about 25 μg, about 70 μg to about 75 μg, about 100 μg to about 125 μg, about 100 μg to about 150 μg, about 125 μg to about 175 μg, about 200 μg to about 250 μg, about 225 μg to about 300 μg, or about 250 μg to about 350 μg of the protein nanostructures.

In some embodiments, the unit dose of the pharmaceutical composition comprises about 0.5 μg to about 1 μg, about 20 μg to about 25 μg, about 25 μg to about 50 μg, about 50 μg to about 70 μg, about 70 μg to about 75 μg, about 75 μg to about 100 μg, about 100 μg to about 125 μg, about 125 μg to about 150 μg, about 150 μg to about 175 μg, about 175 μg to about 200 μg, about 200 μg to about 250 μg, or about 250 μg to about 300 μg of the protein nanostructures.

### Definitions

The singular forms “a,” “an,” and “the” are intended to include the plural forms as well, unless the context clearly indicates otherwise.

As used herein, the term “about” means a range of values including the specified value, which a person of ordinary skill in the art would consider reasonably similar to the specified value. For example, about means within a standard deviation using measurements generally acceptable in the art. For example, about means a range extending to +/−10%, +/−5%, +/−3%, or +/−1% of the specified value.

The term “at least” followed by a number is used herein to denote the start of a range beginning with that number (which may be a range having an upper limit or no upper limit, depending on the variable being defined). For example, “at least 1” means 1 or more than 1.

The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number)” this means a range whose lower limit is the first number and whose upper limit is the second number. For example, 25 to 100 mm means a range whose lower limit is 25 mm, and whose upper limit is 100 mm.

The term “identical” or percent “identity,” in the context of two or more nucleic acid or polypeptide sequences, refers to two or more sequences or subsequences that are the same or have a specified percentage of amino acid residues or nucleotides that are the same, when compared and aligned for maximum correspondence. Methods of alignment of sequences for comparison are well known in the art. Once aligned, the number of matches is determined by counting the number of positions where an identical nucleotide or amino acid residue is present in both sequences. The percent sequence identity is determined by dividing the number of matches in the alignment by the length of the reference sequence, followed by multiplying the resulting value by 100. For example, a peptide sequence that has 1166 matches when aligned with a reference sequence having 1554 amino acids is 75.0 percent identical to the test sequence (1166÷1554*100=75.0). As the terms are used herein, gaps in the alignment do not decrease the percent sequence identity. Unless otherwise specified, optimal alignment of sequences for comparison is conducted by the global alignment algorithm of Needleman and Wunsch, Mol. Biol. 48:443 (1970) as implemented by EMBOSS Needle (on the World Wide Web at ebi.ac.uk/Tools/psa/emboss_needle/) (Madeira et al. Nucleic Acids Res. 50(W1):W276-W279 (2022)). Other alignment methods may be used, including without limitation those described in Devereux, et al, Nucleic Acids Res. 12:387-95 (1984); Atschul et al. J. Mo. Biol. 215:403-10 (1990) (BLAST); Carrillo and Lipman Siam J. Appl. Math. 48(5) (1988); Computational Molecular Biology (Lesk, AM, ed., 1989); Biocomputing Informatics and Genome Projects, (Smith, DW, ed., 1993); Computer Analysis of Sequence Data, Part I, (Griffin and Griffin, eds., 1994); Sequence Analysis in Molecular Biology (von Heinje, 2012); Sequence Analysis Primer (Gribskov and Devereux, J., eds. 1993). Sequence identity is calculated using the implementation of the Needleman-Wunsch algorithm provided by the National Library of Medicine (on the World Wide Web at blast.ncbi.nlm.nih.gov/Blast.cgi?PAGE_TYPE=BlastSearch&BLAST_SPEC=GlobalAln).

For example, sequence identity can be determined by standard methods that are commonly used to compare the similarity of two polypeptide or two polynucleotide sequences. Using a computer program such as EMBOSS Needle or BLAST, two polypeptide or two polynucleotide sequences are aligned for optimal matching of their respective residues (either along the full length of one or both sequences, or along a pre-determined portion of one or both sequences). The programs provide a default opening penalty and a default gap penalty, and a scoring matrix such as PAM 250 (a standard scoring matrix; see Dayhoff et al., in Atlas of Protein Sequence and Structure, vol. 5, supp. 3 (1978)) that can be used in conjunction with the computer program.

The term “substantially similar” refers to two polypeptides, proteins, assemblies, nanostructures, or other physical embodiments of the present that may differ in architecture, sequence, configuration, associations, and the like yet provide about the same or similar properties, structure, activity, and/or function. For example, a nanostructure having an 153 architecture and/or a quaternary structure provides properties, activity and/or function as nanostructures having about the same or similar to a nanostructure having the I53-50 architecture and/or a quaternary structure. In other words, embodiments of the present disclosure may be exchanged, yet achieve a desired outcome, e.g., in properties, structures, activities, and/or functions.

Throughout this specification and the claims which follow, unless the context requires otherwise, the word “comprise”, and variations such as “comprises” and “comprising,” as well as “has” or “having” and “includes” or “including,” will be understood to imply the inclusion of a stated element or step or group of elements or steps but not the exclusion of any other element or step or group of elements or steps. “Consisting essentially of” or “consists essentially” indicates exclusion of elements or steps that materially affect the basic and novel characteristics of the claimed invention.

Any aspect or embodiment described herein can be combined with any other aspect or embodiment as disclosed herein.

### Examples

Characteristics that make a particular nanomaterial useful include modularity, spontaneous self-assembly across a useful range of concentrations, stability, accessible termini, and particle size. Circular permutation is one method for changing the accessibility of termini. Alternatively, de novo designed termini extensions that are well ordered can also change termini accessibility. Here we have used both techniques to change the termini availability of the protein nanomaterial I53-50 and demonstrated the utility of these new constructs by displaying the Borrelia burgdorferi sensu lato antigen OspA.

### Example 1: Extension of C Terminus of I53-50A to Make C Terminus Surface-Accessible

This Example demonstrates computational design of a I53-50A variant (termed “CompAext”) in which the C terminus of the protein is accessible on the surface of the trimeric component, which enables polypeptide fusion at the N terminus of the protein to be displayed, such as an antigen. After modelling this extension, contacts between the extension and adjacent segments were improved by remodeling the protein. This resulted in extensive remodeling of the N terminus residues of I53-50A, and modification of residues scattered throughout the primary structure (sequence) of I53-50A.

A de novo helical segment was designed off of the C terminus using RFDesign inpainting. Adjacent segment loop lengths were preserved, but still allowed to be designed. This enables optimization of the original loop with any contacting de novo segments. De novo segment lengths between 1 and 30 amino acids were sampled. To build the final helical segment Rfdiffusion was used. The de novo segment and adjacent regions were allowed to diffuse. Where de novo elements were introduced a range of lengths were sampled around the lengths identified by inpainting. The highest scoring designs from RFdiffusion were selected for sequence design with ProteinMPNN. Structures for top sequences were predicted with ColabFold and compared to the design model. The result of the design process was polypeptide sequences as disclosed in Table 1. A flexible linker sequence was included at the C terminus of each design to further facilitate fusion of other polypeptides (e.g., an antigen or purification tag) to the C terminus. This C-terminal linker and N-terminal leader sequences are underlined; the underline sequences are optional as they could be replaced with other linkers and leaders.

The resulting constructs are the sequences in Table 2, below, that have ‘None’ in the permutation column.

The extension for each design is listed in Table 1.

TABLE 1
Extending Polypeptide Segments for C-terminal
Extensions

| | | SEQ |
|---|---|---|
| Name | Extension | ID NO: |
| CompA.005 | ENHARFAALRAELAGT | 33 |
| CompA.006 | ENPELTKEVAAFLAGT | 34 |
| CompA.009 | EYSEQFEARKKKLEGT | 35 |
| CompA.010 | RIDEEYQKRLEKLRGT | 36 |
| CompA.011 | KYKEQLDKQLKLQLGT | 37 |
| CompA.012 | KEKEYFEEQLEKLKGT | 38 |
| CompA.013 | YSKARLAEIKKALAGT | 39 |
| CompA.014 | AADEHMAAIMAALKGT | 40 |
| CompA.015 | VGDKLLAELKAQLAGT | 41 |
| CompA.016 | YLKANAEKLHKLLAGT | 42 |
| CompA.017 | ILAKLKAKILAKLKGT | 43 |
| CompA.018 | YSQATLKEILKALAGT | 44 |
| CompA.025 | FSKEACKKAILETKDGSGSGT | 45 |
| CompA.029 | PEVQAVHKKALAVAPKGT | 46 |
| CompA.030 | EEVKAVQQKALALAPKLTGSGSGT | 47 |
| CompA.031 | AEVAANQAKALSLAPPEAGGT | 48 |
| CompA.032 | AEVEANQAKALSLAPAPAGSGSGT | 49 |
| CompA.033 | EEVEAVQKKALSIAEELEGSGSGT | 50 |
| CompA.034 | EEVAAVQKKALSLAPKEPSGT | 51 |
| CompA.035 | DTLALLKERKGT | 52 |
| CompA.036 | DSMAILEKVKGT | 53 |
| CompA.037 | NQMELVKKVFGT | 54 |
| CompA.038 | NAMELVKKALGT | 55 |
| CompA.039 | NQMELVKKAEGT | 56 |
| CompA.040 | DSMELFKKAEGT | 57 |
| CompA.041 | DAMELLKEAEAIMGT | 58 |
| CompA.042 | DPMKLVEEVEKLLGT | 59 |
| CompA.043 | DPMAEVEKAKALEGT | 60 |
| CompA.044 | DPMALVDKVLALFGT | 61 |

### Example 2: Permutation of CompAext

This Example describes circular permutation of I53-50. Various permutations were modelled computationally. The preferred results involved circular permutation of CompAext. Conceptually, first, the C terminus of I53-50Aext was connected to its N terminus, to generate a circular polypeptide chain using an extending polypeptide segment. Second, breakpoints between the secondary structure elements were identified to create a N terminus. Three preferred breakpoints were identified, at approximately residues 73, 106, and 127. These permutations were termed, respectively, Permutation 1, Permutation 2, and Permutation 3. Lastly, computational modelling was applied to design novel contacts between secondary structure elements within the tertiary structure of the Permutation 1, Permutation 2, and Permutation 3. Resulting sequences are provided in Table 2.

### Permutation of Structural Elements:

I53-50 CompA (or “I53-50A”) is a TIM barrel fold derived from the protein 1WA3. TIM barrels are an approximately circular repeat protein consisting of eight pairs of beta-strands and alpha-helices. The beta-strands are parallel and oriented around a solvated central lumen, with the helices are on the external surface. The lumen is often capped on one or both ends with an additional, terminal helical segment. This is true for CompA, which has an N-terminal capping helix (H1). Therefore, CompA secondary structure elements are, in order from N to C terminus, H1, E1, H2, E2, . . . , H8, E8, H9. Because of the structure of TIM barrels, the protein can be divided into pairs of secondary structure elements and recombined in any order simply by designing new connecting loops. However, some connectivity pairs are more likely to successfully fold into the desired structure than others. 1WA3 is also a C3-symmetric homotrimer and much of the interface is formed by loops between strands and helices which further limits the number of possible connections. Applying those limitations, the order of the helices and strands within the peptide sequence where permuted with the further constraint that helices and strands must alternate, resulting in 3474 possible permutations.

To evaluate these permutations, loops were closed using RFDesign inpainting. When permuted segments remain adjacent to its original neighboring segments, the connecting loop length is preserved, but still allowed to be designed. This enables optimization of the original loop with any contacting de novo loops. Where the order is not the same as the original sequence loop lengths between 1 and 30 amino acids were sampled.

### Selection of Permuted Elements

Most permutations resulted in poor quality loop closures or no viable solution could be found. Of the closed permutations, the simplest permutation, (i.e., connecting the C terminus to the N terminus and then introducing a cut point elsewhere in the sequence) produced the highest scoring results. Permutations with a minimum 1ddt>0.75 were selected. Some of these permutations introduced irreconcilable clashes with either CompB or symmetric copies of CompA in the I53-50 assembly and were discarded.

### Loop and Termini Design

To build the final loops Rfdiffusion was used. Loops and adjacent regions were allowed to diffuse. Where de novo elements were introduced a range of lengths were sampled around the lengths identified by inpainting. The highest scoring designs from RFdiffusion were selected for sequence design with ProteinMPNN. Structures for top sequences were predicted with ColabFold and compared to the design model. The result of the design process was polypeptide sequences as disclosed in Table 2. A linker was included at the C terminus of each design to further facilitate fusion of other polypeptides (e.g., an antigen) to the C terminus. This C-terminal linker and N-terminal leader sequences are underlined; the underline sequences are optional as they could be replaced with other linkers and leaders.

TABLE 2
Circular Permutations and C-terminal Extensions

| Name | Permutation | AA Sequence |
|---|---|---|
| CompA.001 | None | MKMEELFKKHKIVAVLRANSVEEAIEKAVAVFAGGVHLIEITFTVP |
| (I53-50A) | | DADTVIKALSVLKEKGAIIGAGTVTSVEQCRKAVESGAEFIVSPHLD |
| | | EEISQFCKEKGVFYMPGVMTPTELVKAMKLGHDILKLFPGEVVGPQ |
| | | FVKAMKGPFPNVKFVPTGGVNLDNVCKWFKAGVLAVGVGKALVK |
| | | GKPDEVREKAKKFVKKIRGCTEGT (SEQ ID NO: 62) |
| CompA.002 | Permutation 1 | MGSGSVEQCRKAVEAGAEYIVSPHLDEEISQFCKEKGIPYMPGVMT |
| | (74-201:1-73) | PTELVKAMKLGHLLLKLFPGEVVGPQFVKAMKKTFPKARFVPTGG |
| | | VNLDNVCEWFKAGVLAVGVGSALVKGTPDEVREKAKAFVEEAEK |
| | | CSEENKKVGEELIKLVTRPEDREMVEIFYKEKIVAVLRANSVEEAIE |
| | | KAVAVFAGGVTIIEITFTVPDADTVIKALSVLKEKGAVIGAGTVTGS |
| | | GT (SEQ ID NO: 63) |
| CompA.003 | Permutation 2 | MGSIPYMPGVMTPTELVKAMKLGHLLLKLFPGEVVGPQFVKAMKK |
| | (107-201:1-106) | TFPKARFVPTGGVNLDNVCEWFKAGVLAVGVGSALVKGTPDEVRE |
| | | KAKAFVEEAAKCSEENKKVGEELIKLVTRPEDREMVEIFYEKKIVA |
| | | VLRANSVEEAIEKAVAVFAGGVTIIEITFTVPDADTVIKALSVLKEKG |
| | | AVIGAGTVTSVEQCRKAVEAGAEYIVSPHLDEEISQFCKEKGSGSGT |
| | | (SEQ ID NO: 64) |
| CompA.004 | Permutation 3 | MGSGHLLLKLFPGEVVGPQFVKAMKKTFPKARFVPTGGVNLDNVC |
| | (128-201:1-127) | EWFKAGVLAVGVGSALVKGTPDEVREKAKAFVEEAAKCSEENKK |
| | | VGEELIKLVTRPEDREMVEIFYKEKIVAVLRANSVEEAIEKAVAVFA |
| | | GGVTIIEITFTVPDADTVIKALSVLKEKGAVIGAGTVTSVEQCRKAV |
| | | EAGAEYIVSPHLDEEISQFCKEKGIPYMPGVMTPTELVKAMKLGSGS |
| | | GT (SEQ ID NO: 65) |
| CompA.005 | None | MGAPADRELLRKLLENRIVAVLRANSVEEAIEKAVAVFAGGVTIIEI |
| | | TFTVPDADTVIKALSVLKEKGAVIGAGTVTSVEQCRKAVEAGAEYI |
| | | VSPHLDEEISQFCKEKGIPYMPGVMTPTELVKAMKLGHLLLKLFPGE |
| | | VVGPQFVKAMKKTYPEARFVPTGGVNLDNVCEWIKAGAIAVGVGS |
| | | ALVKGTPDEVREKAKAFVEEAAKCAAENHARFAALRAELAGT |
| | | (SEQ ID NO: 66) |
| CompA.006 | None | MGAPEEKKMIALLAENPIVAVLRANSVEEAIEKAVAVFAGGVTIIEI |
| | | TFTVPDADTVIKALSVLKEKGAVIGAGTVTSVEQCRKAVEAGAEYI |
| | | VSPHLDEEISQFCKEKGIPYMPGVMTPTELVKAMKLGHLLLKLFPGE |
| | | VVGPQFVKAMKKTYPEARFVPTGGVNLDNVCEWIKAGALAVGVG |
| | | SALVKGTPDEVREKAKAFVEEALKCRGENPELTKEVAAFLAGT |
| | | (SEQ ID NO: 67) |
| CompA.007 | Permutation 1 | MGSGSVEQCRKAVEAGAEYIVSPHLDEEISQFCKEKGVPYMPGVMT |
| | (74-201:1-73) | PTELVKAMKLGHLLLKLFPGEVVGPQFVKAMKKTFPDAAFVPTGG |
| | | VNLDNVCEWFKAGVLAVGVGSALVKGTPDEVREKAKAFVEKAKK |
| | | CDEVNKEVGKKLLLLVTDPADKKMVERFYKEKIVAVLRANSVEEA |
| | | IEKAVAVFAGGVTIIEITFTVPDADTVIKALSVLKEKGAIIGAGTVTG |
| | | SGT (SEQ ID NO: 68) |
| CompA.008 | Permutation 2 | MGSGVPYMPGVMTPTELVKAMKLGHLLLKLFPGEVVGPQFVKAM |
| | (107-201:1-106) | KKTFPDAAFVPTGGVNLDNVCEWFKAGVLAVGVGSALVKGTPDE |
| | | VREKAKAFVEKAKKCDQVNNEVGKKLLLLVTDPADKKMVERFYE |
| | | EKIVAVLRANSVEEAIEKAVAVFAGGVTIIEITFTVPDADTVIKALSV |
| | | LKEKGAIIGAGTVTSVEQCRKAVEAGAEYIVSPHLDEEISQFCKEKG |
| | | SGSGT (SEQ ID NO: 69) |
| CompA.009 | None | MGDPKELAMLKAFLEEKIVAVLRANSVEEAIEKAVAVFAGGVKIIEI |
| | | TFTVPDADTVIKALSVLKEKGAIIGAGTVTSVEQCRKAVEAGAEYIV |
| | | SPHLDEEISQFCKEKGVPYMPGVMTPTELVKAMKLGHQLLKLFPGE |
| | | VVGPQFVKAMKKTYPDAAFVPTGGVNLDNVCEWFKAGATAVGVG |
| | | SALVKGTPDEVREKAKAFVEKAAKCEGEYSEQFEARKKKLEGT |
| | | (SEQ ID NO: 70) |
| CompA.010 | None | MPEKEREIMIAFLKNRIVAVLRANSVEEAIEKAVAVFAGGVKIIEITF |
| | | TVPDADTVIKALSVLKEKGAIIGAGTVTSVEQCRKAVEAGAEYIVSP |
| | | HLDEEISQFCKEKGVPYMPGVMTPTELVKAMKLGHRLLKLFPGEV |
| | | VGPQFVKAMKKTYPDAAFVPTGGVNLDNVCEWFDAGAVAVGVGS |
| | | ALVKGTPDEVREKAKAFVEKAAKCRARIDEEYQKRLEKLRGT (SEQ |
| | | ID NO: 71) |
| CompA.011 | None | MGSEADLKMLKKLYEEKIVAVLRANSVEEAIEKAVAVFAGGVKIIEI |
| | | TFTVPDADTVIKALSVLKEKGAIIGAGTVTSVEQCRKAVEAGAEYIV |
| | | SPHLDEEISQFCKEKGVPYMPGVMTPTELVKAMKLGHKLLKLFPGE |
| | | VVGPQFVKAMKKTYPEAAFVPTGGVNLDNVCEWIEAGAVAVGVG |
| | | SALVKGTPDEVREKAKAFVEKANECAGKYKEQLDKQLKLQLGT |
| | | (SEQ ID NO: 72) |
| CompA.012 | None | MGLPEVELKMIEKIMEEGIVAVLRANSVEEAIEKAVAVFAGGVKIIEI |
| | | TFTVPDADTVIKALSVLKEKGAIIGAGTVTSVEQCRKAVEAGAEYIV |
| | | SPHLDEEISQFCKEKGVPYMPGVMTPTELVKAMKLGHVLLKLFPGE |
| | | VVGPQFVKAMKKTYPNVAFVPTGGVNLDNVCEWIEAGAAAVGVG |
| | | SALVKGTPDEVREKAKAFVEKANECRAKEKEYFEEQLEKLKGT |
| | | (SEQ ID NO: 73) |
| CompA.013 | None | MGVDEKDLKLLEALAANRIVAVLRANSVEEAIEKAVAVFAGGVTII |
| | | EITFTVPDADTVIKALSVLKEKGAIIGAGTVTSVEQCRKAVEAGAEY |
| | | IVSPHLDEEISQFCKEKGVPYMPGVMTPTELVKAMKLGHLLLKLFP |
| | | GEVVGPQFVKAMKKTYPTAAFVPTGGVNLDNVCEWLKAGAVAVG |
| | | VGSALVKGTPDEVREKAKAFVAKADEYAKYSKARLAEIKKALAGT |
| | | (SEQ ID NO: 74) |
| CompA.014 | None | MGVSEKEIEMLKKFNEARIVAVLRANSVEEAIEKAVAVFAGGVTIIE |
| | | ITFTVPDADTVIKALSVLKEKGAIIGAGTVTSVEQCRKAVEAGAEYI |
| | | VSPHLDEEISQFCKEKGVPYMPGVMTPTELVKAMKLGHLLLKLFPG |
| | | EVVGPQFVKAMKKTYPLAAFVPTGGVNLDNVCEWLEAGCIAVGV |
| | | GSALVKGTPDEVREKAKAFVAKARECAAAADEHMAAIMAALKGT |
| | | (SEQ ID NO: 75) |
| CompA.015 | None | MGLSPAEQAMLLAVVENRIVAVLRANSVEEAIEKAVAVFAGGVTII |
| | | EITFTVPDADTVIKALSVLKEKGAIIGAGTVTSVEQCRKAVEAGAEY |
| | | IVSPHLDEEISQFCKEKGVPYMPGVMTPTELVKAMKLGHLLLKLFP |
| | | GEVVGPQFVKAMKKTYPGVAFVPTGGVNLDNVCEWLEAGAAAVG |
| | | VGSALVKGTPDEVREKAKAFVAKADEMGAVGDKLLAELKAQLAG |
| | | T (SEQ ID NO: 76) |
| CompA.016 | None | MGYPEAQIELLDKVIKEGIVAVLRANSVEEAIEKAVAVFAGGVTIIEI |
| | | TFTVPDADTVIKALSVLKEKGAIIGAGTVTSVEQCRKAVEAGAEYIV |
| | | SPHLDEEISQFCKEKGVPYMPGVMTPTELVKAMKLGHLLLKLFPGE |
| | | VVGPQFVKAMKKTYPGAAFVPTGGVNLDNVCEWLKAGAAAVGV |
| | | GSALVKGTPDEVREKAKAFVAKAKECSKYLKANAEKLHKLLAGT |
| | | (SEQ ID NO: 77) |
| CompA.017 | None | MGLSEKEIAIIEAFLENPIVAVLRANSVEEAIEKAVAVFAGGVTIIEIT |
| | | FTVPDADTVIKALSVLKEKGAIIGAGTVTSVEQCRKAVEAGAEYIVS |
| | | PHLDEEISQFCKEKGVPYMPGVMTPTELVKAMKLGHLLLKLFPGEV |
| | | VGPQFVKAMKKTYPDVAFVPTGGVNLDNVCEWLEAGAVAVGVGS |
| | | ALVKGTPDEVREKAKAFVAKAGEKAAILAKLKAKILAKLKGT (SEQ |
| | | ID NO: 78) |
| CompA.018 | None | MGVSEADLALLKALAENQIVAVLRANSVEEAIEKAVAVFAGGVTII |
| | | EITFTVPDADTVIKALSVLKEKGAIIGAGTVTSVEQCRKAVEAGAEY |
| | | IVSPHLDEEISQFCKEKGVPYMPGVMTPTELVKAMKLGHLLLKLFP |
| | | GEVVGPQFVKAMKKTYPSAAFVPTGGVNLDNVCEWLKAGCIAVG |
| | | VGSALVKGTPDEVREKAKAFVAKADECAKYSQATLKEILKALAGT |
| | | (SEQ ID NO: 79) |
| CompA.019 | Permutation 1 | MGSGSVEQCRKAVEAGAEYIVSPHLDEEISQFCKEKGIPYMPGVMT |
| | (74-201:1-73) | PTELVKAMKLGHRVLKLFPGEVVGPQFVKAMKKTFPDARFVPTGG |
| | | VNLDNVCEWFKAGVLAVGVGSALVKGTPDEVREKAKAFVEKAAE |
| | | CSEENEKEGKKALKLETDPAMKKMVKIFYKEKIVAVLRANSVEEAI |
| | | EKAVAVFAGGVTVIEITFTVPDADTVIKALSVLKEKGAVIGAGTVTG |
| | | SGT (SEQ ID NO: 80) |
| CompA.020 | Permutation 2 | MGSGIPYMPGVMTPTELVKAMKLGHRVLKLFPGEVVGPQFVKAM |
| | (107-201:1-106) | KKTFPDARFVPTGGVNLDNVCEWFKAGVLAVGVGSALVKGTPDEV |
| | | REKAKAFVEKAAECSEENEKEGKKALKLETDPAMKKMVKIFYKEK |
| | | IVAVLRANSVEEAIEKAVAVFAGGVTVIEITFTVPDADTVIKALSVL |
| | | KEKGAVIGAGTVTSVEQCRKAVEAGAEYIVSPHLDEEISQFCKEKGS |
| | | GSGT (SEQ ID NO: 81) |
| CompA.021 | Permutation 3 | MGSGGHRVLKLFPGEVVGPQFVKAMKKTFPDARFVPTGGVNLDNV |
| | (128-201:1-127) | CEWFKAGVLAVGVGSALVKGTPDEVREKAKAFVEKAAECSEENEK |
| | | EGKKALKLETDPAMKKMVKIFYKEKIVAVLRANSVEEAIEKAVAVF |
| | | AGGVTVIEITFTVPDADTVIKALSVLKEKGAVIGAGTVTSVEQCRKA |
| | | VEAGAEYIVSPHLDEEISQFCKEKGIPYMPGVMTPTELVKAMKLGS |
| | | GSGT (SEQ ID NO: 82) |
| CompA.022 | Permutation 1 | MGSGSVEQCRKAVEAGAEYIVSPHLDEEISQFCKEKGIPYMPGVMT |
| | (74-201:1-73) | PTELVKAMKLGHKLLKLFPGEVVGPQFVKAMKKTFPDAAFVPTGG |
| | | VNLDNVCEWFKAGVLAVGVGSALVKGTPDEVREKAKAFVEKAKE |
| | | CSEEADKESEKLIKLETDPAMLRMAKIFGKEKIVAVLRANSVEEAIE |
| | | KAVAVFAGGVTVIEITFTVPDADTVIKALSVLKEKGAVIGAGTVTGS |
| | | GT (SEQ ID NO: 83) |
| CompA.023 | Permutation 2 | MGSGIPYMPGVMTPTELVKAMKLGHKLLKLFPGEVVGPQFVKAM |
| | (107-201:1-106) | KKTFPDAAFVPTGGVNLDNVCEWFKAGVLAVGVGSALVKGTPDE |
| | | VREKAKAFVEKAEECSSEAKKESEKLIKLETDPAMLRMAKIFGKEKI |
| | | VAVLRANSVEEAIEKAVAVFAGGVTVIEITFTVPDADTVIKALSVLK |
| | | EKGAVIGAGTVTSVEQCRKAVEAGAEYIVSPHLDEEISQFCKEKGSG |
| | | SGT (SEQ ID NO: 84) |
| CompA.024 | Permutation 3 | MGSGGHKLLKLFPGEVVGPQFVKAMKKTFPDAAFVPTGGVNLDNV |
| | (128-201:1-127) | CEWFKAGVLAVGVGSALVKGTPDEVREKAKAFVEKAEECSEEAKK |
| | | ESEKLIKLETDPAMLRMAKIFGKEKIVAVLRANSVEEAIEKAVAVFA |
| | | GGVTVIEITFTVPDADTVIKALSVLKEKGAVIGAGTVTSVEQCRKAV |
| | | EAGAEYIVSPHLDEEISQFCKEKGIPYMPGVMTPTELVKAMKLGSGS |
| | | GT (SEQ ID NO: 85) |
| CompA.025 | None | MGDKAMASMAKQFCKNKIVAVLRANSVEEAIEKAVAVFAGGVAII |
| | | EITFTVPDADTVIKALSVLKEKGASIGAGTVTSVEQCRKAVEAGAD |
| | | YIVSPHLDEEISQFCKEKGVPYMPGVMTPTELVKAMKLGHVILKLFP |
| | | GEVVGPQFVKAMKKTFPNAQFVPTGGVNLDNVCEWFKAGVLAVG |
| | | VGSALVKGTPDEVREKAKAFVEKVKECAHFSKEACKKAILETKDGS |
| | | GSGT (SEQ ID NO: 86) |
| CompA.026 | Permutation 1 | MGSGSVEQCRKAVEAGADYIVSPHLDEEISQFCKEKGVAYMPGVM |
| | (74-201:1-73) | TPTELVKAMKLGHLVLKLFPGEVVGPQFVKAMKKTFPDVFFVPTG |
| | | GVNLDNVCEWFKAGVLAVGVGSALVKGTPDEVREKAKAFVDKVI |
| | | ACYGPEVQAVHKKALAVAPKLTEAQALMLKAFVEEKIVAVLRANS |
| | | VEEAIEKAVAVFAGGVNIIEITFTVPDADTVIKALSVLKEKGAIIGAG |
| | | TVTGSGT (SEQ ID NO: 87) |
| CompA.027 | Permutation 2 | MGSGVAYMPGVMTPTELVKAMKLGHLVLKLFPGEVVGPQFVKAM |
| | (107-201:1-106) | KKTFPDVFFVPTGGVNLDNVCEWFKAGVLAVGVGSALVKGTPDEV |
| | | REKAKAFVDKVTACYGPEVEAVHEKALAVAPKLTEAQALMLKAF |
| | | VEEKIVAVLRANSVEEAIEKAVAVFAGGVNIIEITFTVPDADTVIKAL |
| | | SVLKEKGAIIGAGTVTSVEQCRKAVEAGADYIVSPHLDEEISQFCKE |
| | | KGSGSGT (SEQ ID NO: 88) |
| CompA.028 | Permutation 3 | MGSSGHLVLKLFPGEVVGPQFVKAMKKTFPDVFFVPTGGVNLDNV |
| | (128-201:1-127) | CEWFKAGVLAVGVGSALVKGTPDEVREKAKAFVDKVTACYGPEV |
| | | EAVHEKALAVAPKLTEAQALMLKAFVEEKIVAVLRANSVEEAIEKA |
| | | VAVFAGGVNIIEITFTVPDADTVIKALSVLKEKGAIIGAGTVTSVEQC |
| | | RKAVEAGADYIVSPHLDEEISQFCKEVGVAYMPGVMTPTELVKAM |
| | | KLGSGSGT (SEQ ID NO: 89) |
| CompA.029 | None | MTEAQALMLKAFVEEKIVAVLRANSVEEAIEKAVAVFAGGVNIIEIT |
| | | FTVPDADTVIKALSVLKEKGAIIGAGTVTSVEQCRKAVEAGADYIVS |
| | | PHLDEEISQFCKEVGVAYMPGVMTPTELVKAMKLGHLVLKLFPGE |
| | | VVGPQFVKAMKKTFPDVFFVPTGGVNLDNVCEWFKAGVLAVGVG |
| | | SALVKGTPDEVREKAKAFVEKVLACIGPEVQAVHKKALAVAPKGT |
| | | (SEQ ID NO: 90) |
| CompA.030 | None | MGTEANALMLKRFVEEKIVAVLRANSVEEAIEKAVAVFAGGVNIIEI |
| | | TFTVPDADTVIKALSVLKEKGAVIGAGTVTSVEQCRKAVEAGAAYI |
| | | VSPHLDEEISQFCKEVGVAYMPGVMTPTELVKAMKLGHLVLKLFP |
| | | GEVVGPQFVKAMKKTFPDAVFVPTGGVNLDNVCEWFKAGVLAVG |
| | | VGSALVKGTPDEVREKAKAFREKVAACDGEEVKAVQQKALALAP |
| | | KLTGSGSGT (SEQ ID NO: 91) |
| CompA.031 | None | MTPAEALMLKRFVKEKIVAVLRANSVEEAIEKAVAVFAGGVNIIEIT |
| | | FTVPDADTVIKALSVLKEKGAIIGAGTVTSVEQCRKAVEAGADYIVS |
| | | PHLDEEISQFCKEVGVAYMPGVMTPTELVKAMKLGHIILKLFPGEV |
| | | VGPQFVKAMKKTFPDAVFVPTGGVNLDNVCEWFKAGVVAVGVGS |
| | | ALVKGTPDEVREKAKAFREKVATCVGAEVAANQAKALSLAPPEAG |
| | | GT (SEQ ID NO: 92) |
| CompA.032 | None | MTPAEALMLKRFVKEKIVA VLRANSVEEAIEKAVAVFAGGVNIIEIT |
| | | FTVPDADTVIKALSVLKEKGAIIGAGTVTSVEQCRKAVEAGADYIVS |
| | | PHLDEEISQFCKEVGVAYMPGVMTPTELVKAMKLGHIILKLFPGEV |
| | | VGPQFVKAMKKTFPDAVFVPTGGVNLDNVCEWFKAGVVAVGVGS |
| | | ALVKGTPDEVREKAKAFREKVATCVGAEVEANQAKALSLAPAPAG |
| | | SGSGT (SEQ ID NO: 93) |
| CompA.033 | None | MTPAEALMLKRFVEEKIVAVLRANSVEEAIEKAVAVFAGGVNIIEIT |
| | | FTVPDADTVIKALSVLKEKGAVIGAGTVTSVEQCRKAVEAGADYIV |
| | | SPHLDEEISQFCKEVGVAYMPGVMTPTELVKAMKLGHILLKLFPGE |
| | | VVGPQFVKAMKETFPDAFFVPTGGVNLDNVCEWFKAGVVAVGVG |
| | | SALVKGTPDEVREKAKAFVEKVNSCTGEEVEAVQKKALSIAEELEG |
| | | SGSGT (SEQ ID NO: 94) |
| CompA.034 | None | MTPAEALMLKRFVEEKIVAVLRANSVEEAIEKAVAVFAGGVNIIEIT |
| | | FTVPDADTVIKALSVLKEKGAVIGAGTVTSVEQCRKAVEAGADYIV |
| | | SPHLDEEISQFCKEVGVAYMPGVMTPTELVKAMKLGHILLKLFPGE |
| | | VVGPQFVKAMKETFPDAFFVPTGGVNLDNVCEWFKAGVVAVGVG |
| | | SALVKGTPDEVREKAKAFKAKVASCTGEEVAAVQKKALSLAPKEP |
| | | SGT (SEQ ID NO: 95) |
| CompA.035 | None | MGNKEEIEEKFAREKIVAVLRANSVEEAIEKAVAVFAGGVGIIEITFT |
| | | VPDADTVIKALSVLKEKGASIGAGTVTSVEQCRKAVEAGAEYIVSP |
| | | HLDEEISQFCKELGIPYMPGVMTPTELVKAMKLGHKVLKLFPGEVV |
| | | GPQFVKAMKKTYPDVLFVPTGGVNLDNVCEWLKAGALAVGVGSA |
| | | LVKGTPDEVREKAKAFVEKVKACGVDTLALLKERKGT (SEQ ID |
| | | NO: 96) |
| CompA.036 | None | MGNEEIEEKFAKEKIVAVLRANSVEEAIEKAVAVFAGGVGIIEITFTV |
| | | PDADTVIKALSVLKEKGASIGAGTVTSVEQCRKAVEAGAEYIVSPHL |
| | | DEEISQFCKELGIPYMPGVMTPTELVKAMKLGHKVLKLFPGEVVGP |
| | | QFVKAMKKTYPEALFVPTGGVNLDNVCEWLKAGAIAVGVGSALV |
| | | KGTPDEVREKAKAFVEKVKACGVDSMAILEKVKGT (SEQ ID NO: |
| | | 97) |
| CompA.037 | None | MGNKEIIEKFAKEKIVAVLRANSVEEAIEKAVAVFAGGVGIIEITFTV |
| | | PDADTVIKALSVLKEKGASIGAGTVTSVEQCRKAVEAGAEYIVSPHL |
| | | DEEISQFCKELGIPYMPGVMTPTELVKAMKLGHKVLKLFPGEVVGP |
| | | QFVKAMKKTYPDALFVPTGGVNLDNVCEWLKAGALAVGVGSALV |
| | | KGTPDEVREKAKAFVEKVKACGVNQMELVKKVFGT (SEQ ID NO: |
| | | 98) |
| CompA.038 | None | MGNVEIIEKFAKEKIVAVLRANSVEEAIEKAVAVFAGGVGIIEITFTV |
| | | PDADTVIKALSVLKEKGASIGAGTVTSVEQCRKAVEAGAEYIVSPHL |
| | | DEEISQFCKELGIPYMPGVMTPTELVKAMKLGHKVLKLFPGEVVGP |
| | | QFVKAMKKTYPDALFVPTGGVNLDNVCEWLKAGALAVGVGSALV |
| | | KGTPDEVREKAKAFVEKVRASGVNAMELVKKALGT (SEQ ID NO: |
| | | 99) |
| CompA.039 | None | MGNPKEIIEKFAKEKIVAVLRANSVEEAIEKAVAVFAGGVGIIEITFT |
| | | VPDADTVIKALSVLKEKGASIGAGTVTSVEQCRKAVEAGAEYIVSP |
| | | HLDEEISQFCKELGIPYMPGVMTPTELVKAMKLGHKVLKLFPGEVV |
| | | GPQFVKAMKKTYPEALFVPTGGVNLDNVCEWFKAGALAVGVGSA |
| | | LVKGTPDEVREKAKAFVEKVKACGVNQMELVKKAEGT (SEQ ID |
| | | NO: 100) |
| CompA.040 | None | MGNKEIGEKFAREKIVAVLRANSVEEAIEKAVAVFAGGVGIIEITFT |
| | | VPDADTVIKALSVLKEKGASIGAGTVTSVEQCRKAVEAGAEYIVSP |
| | | HLDEEISQFCKELGIPYMPGVMTPTELVKAMKLGHKVLKLFPGEVV |
| | | GPQFVKAMKKTYPEALFVPTGGVNLDNVCEWLEAGALAVGVGSA |
| | | LVKGTPDEVREKAKAFVEKVKACPFDSMELFKKAEGT (SEQ ID NO: |
| | | 101) |
| CompA.041 | None | MGDLKMAKAFAREKIVAVLRANSVEEAIEKAVAVFAGGVGIIEITFT |
| | | VPDADTVIKALSVLKEKGAKIGAGTVTSVEQCRKAVEAGADYIVSP |
| | | HLDEEISQFCKELGIPYMPGVMTPTELVKAMKLGHTILKLFPGEVVG |
| | | PQFVKAMKKTYPEALFVPTGGVNLDNVCEWLEAGALAVGVGSAL |
| | | VKGTPDEVREKAKAFVAKVAACPVDAMELLKEAEAIMGT (SEQ ID |
| | | NO: 102) |
| CompA.042 | None | MGDKKMAKAFAKEKIVAVLRANSVEEAIEKAVAVFAGGVGIIEITF |
| | | TVPDADTVIKALSVLKEKGAKIGAGTVTSVEQCRKAVEAGADYIVS |
| | | PHLDEEISQFCKELGIPYMPGVMTPTELVKAMKLGHTILKLFPGEVV |
| | | GPQFVKAMKKTYPQALFVPTGGVNLDNVCEWLEAGALAVGVGSA |
| | | LVKGTPDEVREKAKAFVAKVAACPYDPMKLVEEVEKLLGT (SEQ |
| | | ID NO: 103) |
| CompA.043 | None | MGTDEKMAKAFAREKIVAVLRANSVEEAIEKAVAVFAGGVGIIEITF |
| | | TVPDADTVIKALSVLKEKGAKIGAGTVTSVEQCRKAVEAGADYIVS |
| | | PHLDEEISQFCKELGIPYMPGVMTPTELVKAMKLGHTILKLFPGEVV |
| | | GPQFVKAMKKTYPQALFVPTGGVNLDNVCEWLKAGAIAVGVGSA |
| | | LVKGTPDEVREKAKAFVAKVAACGVDPMAEVEKAKALEGT (SEQ |
| | | ID NO: 104) |
| CompA.0 | None | MGDEKMAKAFAREKIVAVLRANSVEEAIEKAVAVFAGGVGIIEITFT |
| 44 | | VPDADTVIKALSVLKEKGAKIGAGTVTSVEQCRKAVEAGADYIVSP |
| | | HLDEEISQFCKELGIPYMPGVMTPTELVKAMKLGHTILKLFPGEVVG |
| | | PQFVKAMKKTYPEALFVPTGGVNLDNVCEWLDAGALAVGVGSAL |
| | | VKGTPDEVREKAKAFVAKVAACPFDPMALVDKVLALFGT (SEQ ID |
| | | NO: 105) |
| CompB | N/A | NQHSHKDYETVRIAVVRARWHAEIVDACVSAFEAAMADIGGDRFA |
| (I53-50B) | | VDVFDVPGAYEIPLHARTLAETGRYGAVLGTAFVVNGGIYRHEFVA |
| | | SAVIDGMMNVQLSTGVPVLSAVLTPHRYRDSDAHTLLFLALFAVK |
| | | GMEAARACVEILAAREKIAA (SEQ ID NO: 27) |
| CompB | N/A | NQHSHKDHETVRIAVVRARWHAEIVDACVSAFEAAMRDIGGDRFA |
| (I53- | | VDVFDVPGAYEIPLHARTLAETGRYGAVLGTAFVVNGGIYRHEFVA |
| 50B.4PosT1) | | SAVINGMMNVQLNTGVPVLSAVLTPHNYDKSKAHTLLFLALFAVK |
| | | GMEAARACVEILAAREKIAA (SEQ ID NO: 26) |

The linking polypeptide segment for each of the permuted designs is listed in Table 3.

TABLE 3
Linking Polypeptide Segments for Circular Permutations
Permutation 1 (74-201:1-73)

| | |
|---|---|
| CompA.002 | KKVGEELIKLVTRPEDR (SEQ ID NO: 8) |
| CompA.007 | KEVGKKLLLLVTDPADK (SEQ ID NO: 106) |
| CompA.022 | DKESEKLIKLETDPAML (SEQ ID NO: 107) |
| CompA.019 | EKEGKKALKLETDPAMK (SEQ ID NO: 108) |
| CompA.026 | VQAVHKKALAVAPKLTEAQA (SEQ ID NO: 109) |

Permutation 2 (107-201:1-106)

| | |
|---|---|
| CompA.003 | ENKKVGEELIKLVTRPEDR (SEQ ID NO: 110) |
| CompA.008 | VNNEVGKKLLLLVTDPADK (SEQ ID NO: 111) |
| CompA.020 | ENEKEGKKALKLETDPAMK (SEQ ID NO: 112) |
| CompA.023 | EAKKESEKLIKLETDPAML (SEQ ID NO: 113) |
| CompA.027 | PEVEAVHEKALAVAPKLTEAQA (SEQ ID NO: 114) |

Permutation 3 (128-201:1-127)

| | |
|---|---|
| CompA.004 | ENKKVGEELIKLVTRPEDR (SEQ ID NO: 110) |
| CompA.021 | ENEKEGKKALKLETDPAMK (SEQ ID NO: 112) |
| CompA.024 | EAKKESEKLIKLETDPAML (SEQ ID NO: 113) |
| CompA.028 | PEVEAVHEKALAVAPKLTEAQA (SEQ ID NO: 114) |

### Example 3: Experimental Verification

Designs with pLDDT>0.90 were ordered as bicistronic plasmids for cytosolic expression in E. coli. In one set of constructs, one open reading frame encoded a CompB (153-50B); the other encoded each of the CompA (153-50A variants) in Table 2, above, with a 6×His tag on the C terminus of CompA. In another set of experiment, one open reading frame encoded a full-length wild-type OspA fused the C terminus of each the CompA (I53-50A variants) in Table 2 (in 5′ to 3′ order, an I53-50A variant, a polypeptide linker, and OspA); and the other open reading frame encoded CompB (I53-50B.4PosT1) having a 6×His tag on the C terminus. Successful designs are expected to assemble into 153-50-derived nanostructures when expressed in E. coli cytosols. Table 2 lists the selected designs. In each sequence in Table 2, the start codon at the beginning of each sequence and the polypeptide linker sequence at the C terminus, used to connect to OspA, is underlined.

### Screening and Characterization

Constructs were screened by expressing in E. coli at 2 ml scale and lysed using sonication. Clarified lysates were purified by Ni-NTA MagBeads. Purification was characterized by SDS-PAGE. Constructs where both components were in the eluate fraction were considered passing, failing where only CompB is in the eluate fraction, and ambiguous where only CompA or not components were observed in the eluate.

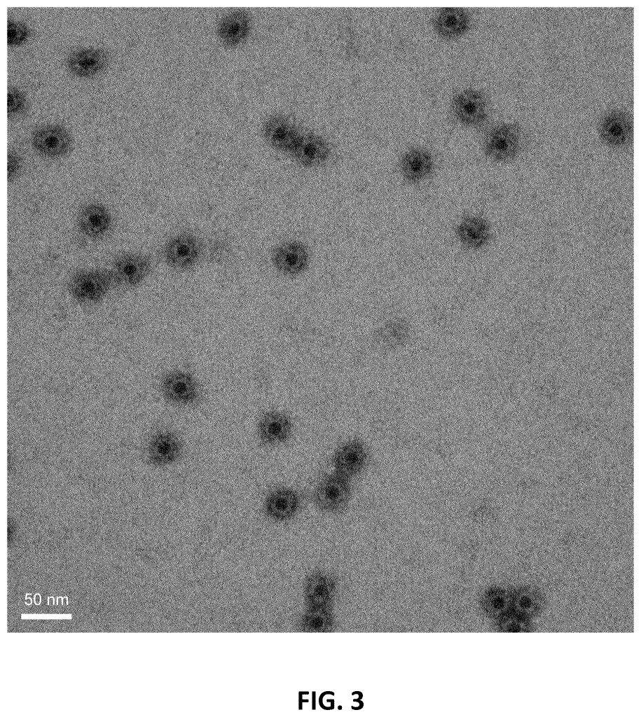

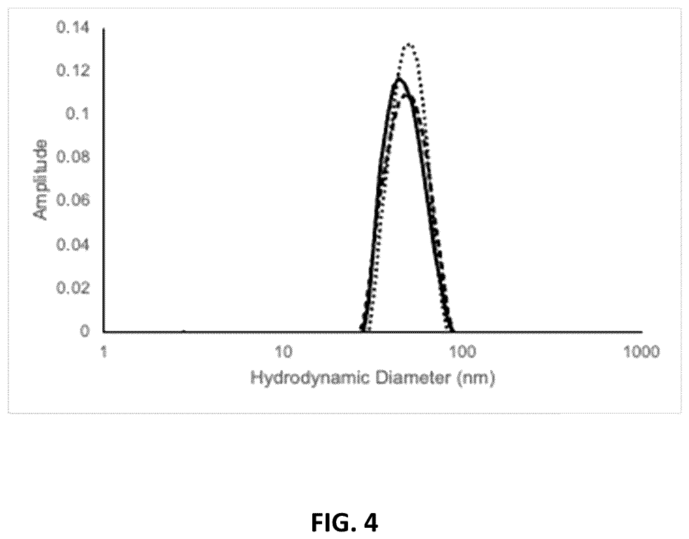

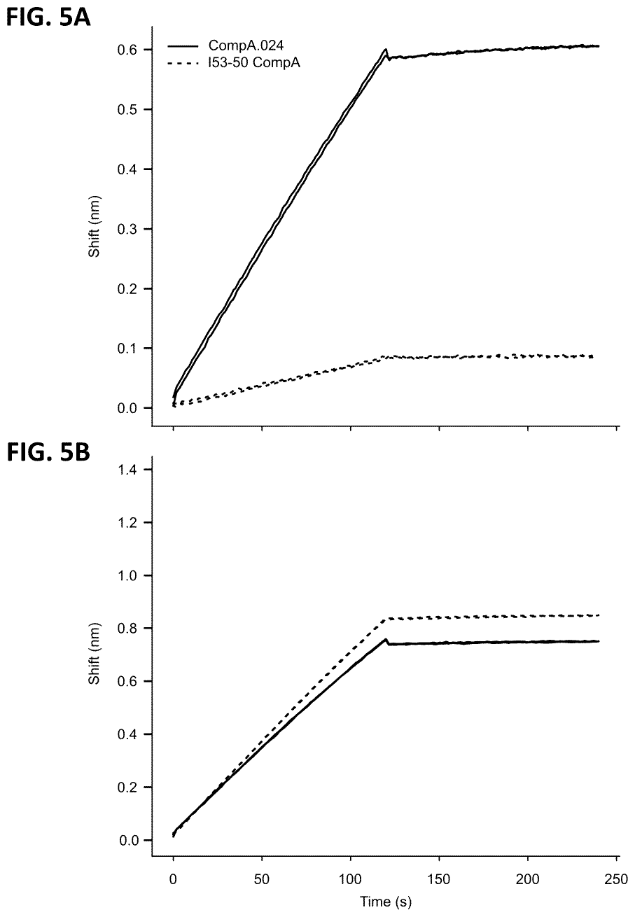

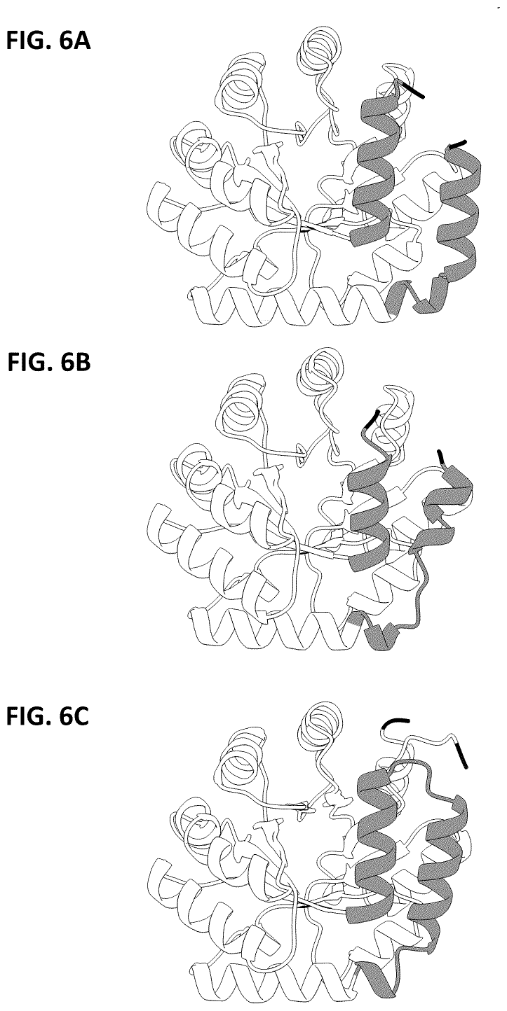

Constructs were further characterized by expression in E. coli at 500 mL scale and lysed using sonication or microfluidization, as bicistronic constructs with CompB, or as monocistronic constructs where the open reading frame only encoded CompA. Clarified lysates were purified over a Ni-NTA gravity column, and further purified by SEC. Purified VLPs were characterized by DLS and negative-stain EM (FIG. 3). Purified CompA was mixed with purified CompB, further purified by SEC to remove excess component, and characterized by DLS (FIG. 4). The impact of circular permutation on antigen accessibility was evaluated by BLI (FIG. 5). An antibody that binds to the C-terminal end of the antigen is occluded when displayed on I53-50 CompA through an antigen C-terminus to CompA N-terminus genetic fusion. When displayed on CompA.024 by an antigen N-terminus to CompA C-terminus genetic fusion, significantly higher antibody binding is observed (FIG. 5A), demonstrating improved epitope accessibility. An antibody that binds an epitope on the middle of the antigen binds equally well to the antigen when displayed on I53-50 CompA or CompA.024 (FIG. 5B). Structural models of representative termini extensions (FIG. 6A, B) and circular permutation (FIG. 6C) are provided.

### In Vitro Assembly Enhancement

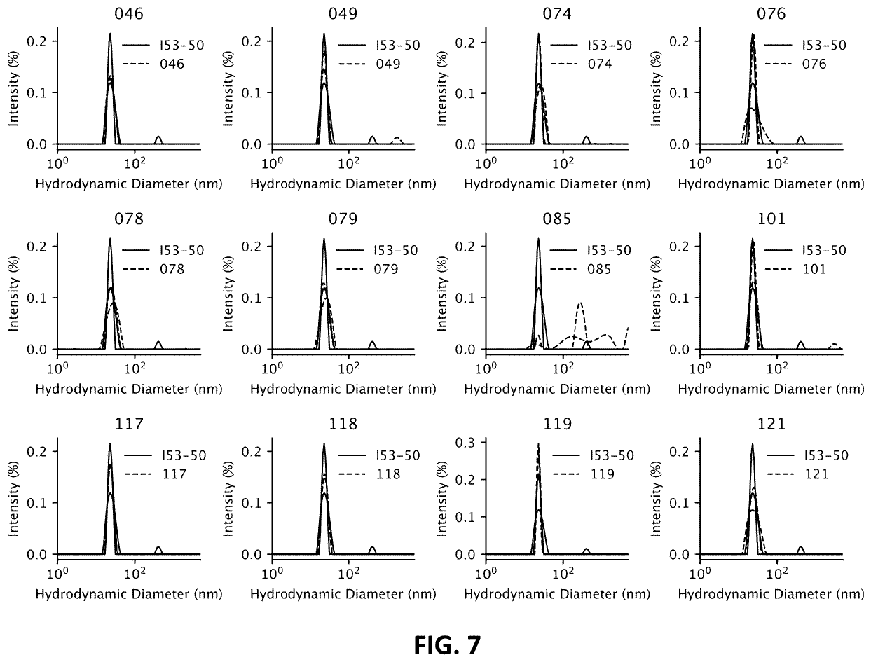

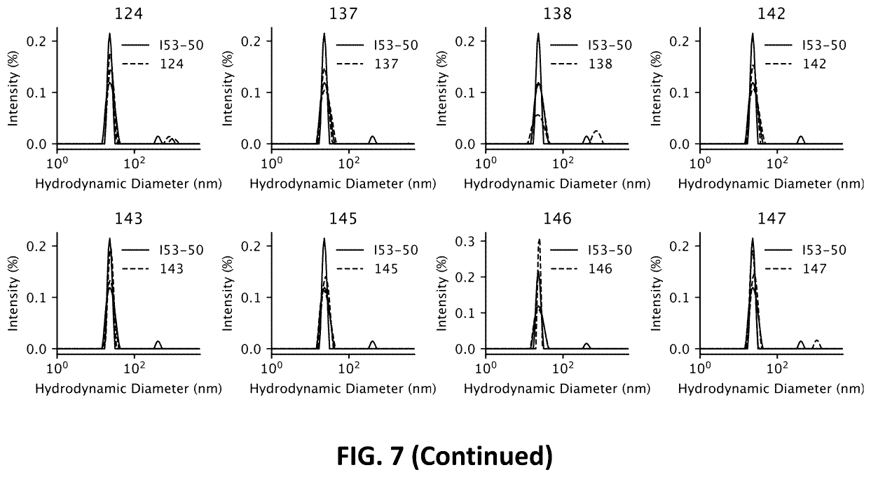

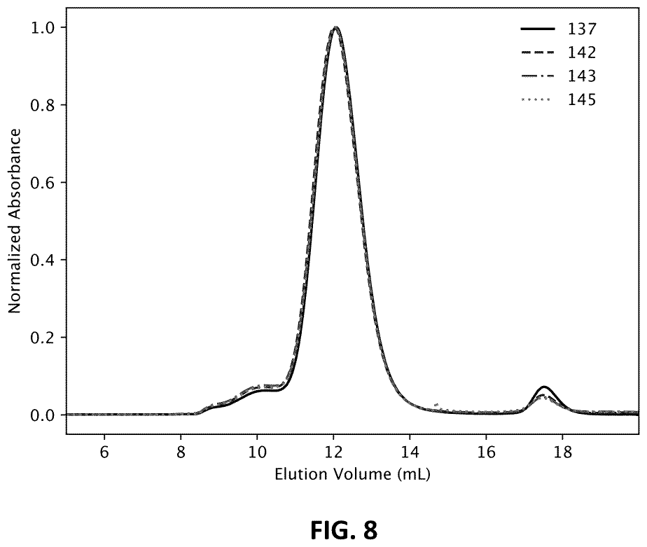

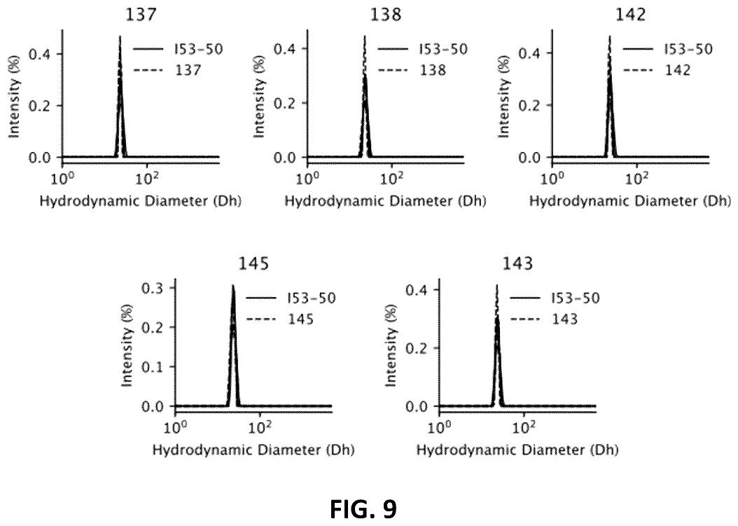

To improve in vitro assembly a second set of designs obtained using multiple approaches was ordered (TABLE 4). These designs were expressed in E. coli as a monocistronic construct and purified by IMAC. CompB was mixed with IMAC eluate and VLP assembly measured by DLS (FIG. 7). A selection of constructs with a size distribution consistent with I53-50 assembly were further purified by SEC. SEC purified constructs were mixed in excess with CompB, and the resulting assemblies purified by SEC using a Superose 6 column. A small excess CompA peak, eluting at ˜17.5 ml, but no excess CompB, was observed in the SEC chromatogram (FIG. 8). Major fractions from the VLP peak (˜12 ml elution volume) were pooled and analyzed by DLS (FIG. 9).

TABLE 4
Additional Circular Permutations and C-terminal Extensions

| Name | Permutation | Sequence | SEQ ID NO |
|---|---|---|---|
| CompA.045 | None | MGSSHHHHHHGSGSEADLKMLKKLYEERIVAVLRANSVE | 115 |
| | | EAIEKAVAVFAGGVKIIEITFTVPDADTVIKALSVLKEKGAII | |
| | | GAGTVTSVEQCRKAVEAGAEYIVSPHLDEEISQFCKEKGVP | |
| | | YMPGVMTPTELVKAMKLGHKLLKLFPGEVVGPQFVKAM | |
| | | KKTYPEAAFVPTGGVNLDNVCEWIEAGAVAVGVGSALVK | |
| | | GTPDEVREKAKAFVEKARLCAGKYKEQLDKQLALQLGT | |
| CompA.046 | None | MGSSHHHHHHGSGSEADLKMLKKLYQERIVAVLRANSVE | 116 |
| | | EAIEKAVAVFAGGVKIIEITFTVPDADTVIKALSVLKEKGAII | |
| | | GAGTVTSVEQCRKAVEAGAEYIVSPHLDEEISQFCKEKGVP | |
| | | YMPGVMTPTELVKAMKLGHKLLKLFPGEVVGPQFVKAM | |
| | | KKTYPEAAFVPTGGVNLDNVCEWIEAGAVAVGVGSALVK | |
| | | GTPDEVREKAKAFVEKANLCAGKYKKQLDKQLALQLGT | |
| CompA.047 | None | MGSSHHHHHHGSGSEADLKMLKKLYTEKIVAVLRANSVE | 117 |
| | | EAIEKAVAVFAGGVKIIEITFTVPDADTVIKALSVLKEKGAII | |
| | | GAGTVTSVEQCRKAVEAGAEYIVSPHLDEEISQFCKEKGVP | |
| | | YMPGVMTPTELVKAMKLGHKLLKLFPGEVVGPQFVKAM | |
| | | KKTYPEAAFVPTGGVNLDNVCEWIEAGAVAVGVGSALVK | |
| | | GTPDEVREKAKAFVQKANECAGKYSDQLDKQLKLQLGT | |
| CompA.048 | None | MGSSHHHHHHGSGSEADLKMLKKLYTEKIVAVLRANSVE | 118 |
| | | EAIEKAVAVFAGGVKIIEITFTVPDADTVIKALSVLKEKGAII | |
| | | GAGTVTSVEQCRKAVEAGAEYIVSPHLDEEISQFCKEKGVP | |
| | | YMPGVMTPTELVKAMKLGHKLLKLFPGEVVGPQFVKAM | |
| | | KKTYPEAAFVPTGGVNLDNVCEWIEAGAVAVGVGSALVK | |
| | | GTPDEVREKAKAFVQKANECAGKYSSQLDKQLSLQLGT | |
| CompA.052 | Permutation | MGSSHHHHHHGSGGHKLLKLFPGEVVGPQFVKAMKKTFP | 119 |
| | 3 | DAAFVPTGGVNLDNVCEWFKAGVLAVGVGSALVKGTPDE | |
| | (128-201:1-127) | VREKAKAFVEKANRCSEEAEKESEKLIKLETDPAMLRMAK | |
| | | IFGKEKIVAVLRANSVEEAIEKAVAVFAGGVTVIEITFTVPD | |
| | | ADTVIKALSVLKEKGAVIGAGTVTSVEQCRKAVEAGAEYI | |
| | | VSPHLDEEISQFCKEKGIPYMPGVMTPTELVKAMKLGSGSG | |
| | | T | |
| CompA.053 | None | MGSSHHHHHHGSTEAQALMLKAFVEEKIVAVLRANSVEE | 120 |
| | | AIEKAVAVFAGGVNIIEITFTVPDADTVIKALSVLKEKGAIIG | |
| | | AGTVTSVEQCRKAVEAGADYIVSPHLDEEISQFCKEVGVA | |
| | | YMPGVMTPTELVKAMKLGHLVLKLFPGEVVGPQFVKAM | |
| | | KKTFPDVFFVPTGGVNLDNVCEWFKAGVLAVGVGSALVK | |
| | | GTPDEVREKAKAFVDKVTACIDAEVNAVHKKALAVAPKG | |
| | | T | |
| CompA.058 | None | MGSSHHHHHHGSGSGNEEIEEKFASEKIVAVLRANSVEEAI | 121 |
| | | EKAVAVFAGGVGIIEITFTVPDADTVIKALSVLKEKGASIGA | |
| | | GTVTSVEQCRKAVEAGAEYIVSPHLDEEISQFCKELGIPYM | |
| | | PGVMTPTELVKAMKLGHKVLKLFPGEVVGPQFVKAMKKT | |
| | | YPEALFVPTGGVNLDNVCEWLKAGAIAVGVGSALVKGTP | |
| | | DEVREKAKAFVEKVSACGVDSMAILSKVKGT | |
| CompA.059 | None | MGSSHHHHHHGSGSGNEEIEEKFAKEKIVAVLRANSVEEAI | 122 |
| | | EKAVAVFAGGVGIIEITFTVPDADTVIKALSVLKEKGASIGA | |
| | | GTVTSVEQCRKAVEAGAEYIVSPHLDEEISQFCKELGIPYM | |
| | | PGVMTPTELVKAMKLGHKVLKLFPGEVVGPQFVKAMKKT | |
| | | YPEALFVPTGGVNLDNVCEWLKAGAIAVGVGSALVKGTP | |
| | | DEVREKAKAFVEKVSACGVDSMAILEKVKGT | |
| CompA.060 | None | MGSSHHHHHHGSGNVEIIEKFAKEKIVAVLRANSVEEAIEK | 123 |
| | | AVAVFAGGVGIIEITFTVPDADTVIKALSVLKEKGASIGAGT | |
| | | VTSVEQCRKAVEAGAEYIVSPHLDEEISQFCKELGIPYMPG | |
| | | VMTPTELVKAMKLGHKVLKLFPGEVVGPQFVKAMKKTYP | |
| | | DALFVPTGGVNLDNVAEWLKAGALAVGVGSALVKGTPDE | |
| | | VREKAKAFVEKVNASGVNAMKLVEKALGT | |
| CompA.061 | None | MGSSHHHHHHGSGNVEIIEKFASEKIVAVLRANSVEEAIEK | 124 |
| | | AVAVFAGGVGIIEITFTVPDADTVIKALSVLKEKGASIGAGT | |
| | | VTSVEQCRKAVEAGAEYIVSPHLDEEISQFCKELGIPYMPG | |
| | | VMTPTELVKAMKLGHKVLKLFPGEVVGPQFVKAMKKTYP | |
| | | DALFVPTGGVNLDNVAEWLKAGALAVGVGSALVKGTPDE | |
| | | VREKAKAFVEKVSASGVNSMELVKKALGT | |
| CompA.062 | None | MGSSHHHHHHGSGNVEIIEKFASEKIVAVLRANSVEEAIEK | 125 |
| | | AVAVFAGGVGIIEITFTVPDADTVIKALSVLKEKGASIGAGT | |
| | | VTSVEQCRKAVEAGAEYIVSPHLDEEISQFCKELGIPYMPG | |
| | | VMTPTELVKAMKLGHKVLKLFPGEVVGPQFVKAMKKTYP | |
| | | DALFVPTGGVNLDNVAEWLKAGALAVGVGSALVKGTPDE | |
| | | VREKAKAFVEKVSASGVNAMELVKKALGT | |
| CompA.065 | None | MGSSHHHHHHGSGNPKEIIEKFASEKIVAVLRANSVEEAIE | 126 |
| | | KAVAVFAGGVGIIEITFTVPDADTVIKALSVLKEKGASIGAG | |
| | | TVTSVEQCRKAVEAGAEYIVSPHLDEEISQFCKELGIPYMP | |
| | | GVMTPTELVKAMKLGHKVLKLFPGEVVGPQFVKAMKKT | |
| | | YPEALFVPTGGVNLDNVCEWFKAGALAVGVGSALVKGTP | |
| | | DEVREKAKAFVEKVSACGVNQMELVKKAEGT | |
| CompA.066 | None | MGSSHHHHHHGSGNPKEIIEKFASEKIVAVLRANSVEEAIE | 127 |
| | | KAVAVFAGGVGIIEITFTVPDADTVIKALSVLKEKGASIGAG | |
| | | TVTSVEQCRKAVEAGAEYIVSPHLDEEISQFCKELGIPYMP | |
| | | GVMTPTELVKAMKLGHKVLKLFPGEVVGPQFVKAMKKT | |
| | | YPEALFVPTGGVNLDNVCEWFKAGALAVGVGSALVKGTP | |
| | | DEVREKAKAFVEKVSACGVNSMELVKKAEGT | |
| CompA.067 | None | MGSSHHHHHHGSGSGNKEIGEKFAEEKIVAVLRANSVEEAI | 128 |
| | | EKAVAVFAGGVGIIEITFTVPDADTVIKALSVLKEKGASIGA | |
| | | GTVTSVEQCRKAVEAGAEYIVSPHLDEEISQFCKELGIPYM | |
| | | PGVMTPTELVKAMKLGHKVLKLFPGEVVGPQFVKAMKKT | |
| | | YPEALFVPTGGVNLDNVCEWLEAGALAVGVGSALVKGTP | |
| | | DEVREKAKAFVEKVNACPFNSMELFRKAEGT | |
| CompA.068 | None | MGSSHHHHHHGSGSGNKEIGEKFATEKIVAVLRANSVEEAI | 129 |
| | | EKAVAVFAGGVGIIEITFTVPDADTVIKALSVLKEKGASIGA | |
| | | GTVTSVEQCRKAVEAGAEYIVSPHLDEEISQFCKELGIPYM | |
| | | PGVMTPTELVKAMKLGHKVLKLFPGEVVGPQFVKAMKKT | |
| | | YPEALFVPTGGVNLDNVCEWLEAGALAVGVGSALVKGTP | |
| | | DEVREKAKAFVEKVRRCPFDSQELFKKAEGT | |
| CompA.069 | None | MGSSHHHHHHGSGSGNKEIGEKFATEKIVAVLRANSVEEAI | 130 |
| | | EKAVAVFAGGVGIIEITFTVPDADTVIKALSVLKEKGASIGA | |
| | | GTVTSVEQCRKAVEAGAEYIVSPHLDEEISQFCKELGIPYM | |
| | | PGVMTPTELVKAMKLGHKVLKLFPGEVVGPQFVKAMKKT | |
| | | YPEALFVPTGGVNLDNVCEWLEAGALAVGVGSALVKGTP | |
| | | DEVREKAKAFVEKVKACPFDSMELFKKAEGT | |
| CompA.070 | None | MGSSHHHHHHGSGSGNKEIGEKFASEKIVAVLRANSVEEAI | 131 |
| | | EKAVAVFAGGVGIIEITFTVPDADTVIKALSVLKEKGASIGA | |
| | | GTVTSVEQCRKAVEAGAEYIVSPHLDEEISQFCKELGIPYM | |
| | | PGVMTPTELVKAMKLGHKVLKLFPGEVVGPQFVKAMKKT | |
| | | YPEALFVPTGGVNLDNVCEWLEAGALAVGVGSALVKGTP | |
| | | DEVREKAKAFVEKVSACPFDSMELFKKAEGT | |
| CompA.074 | None | MGSSHHHHHHGSGSGNEEMEELFASHKIVAVLRANSVEEA | 132 |
| | | IEKAVAVFAGGVHLIEITFTVPDADTVIKALSVLKEKGAIIG | |
| | | AGTVTSVEQCRKAVESGAEFIVSPHLDEEISQFCKEKGVFY | |
| | | MPGVMTPTELVKAMKLGHKVLKLFPGEVVGPQFVKAMK | |
| | | GPFPEVLFVPTGGVNLDNVCEWLKAGVIAVGVGSALVKGT | |
| | | PDEVREKAKAFVEKVSACGVDSMAILSKVKGT | |
| CompA.075 | None | MGSSHHHHHHGSGSGNEEMEELFAKHKIVAVLRANSVEE | 133 |
| | | AIEKAVAVFAGGVHLIEITFTVPDADTVIKALSVLKEKGAII | |
| | | GAGTVTSVEQCRKAVESGAEFIVSPHLDEEISQFCKEKGVF | |
| | | YMPGVMTPTELVKAMKLGHKVLKLFPGEVVGPQFVKAM | |
| | | KGPFPEVLFVPTGGVNLDNVCEWLKAGVIAVGVGSALVK | |
| | | GTPDEVREKAKAFVEKVSACGVDSMAILEKVKGT | |
| CompA.076 | None | MGSSHHHHHHGSGSGNEEMEELFASHKIVAVLRANSVEEA | 134 |
| | | IEKAVAVFAGGVHLIEITFTVPDADTVIKALSVLKEKGAIIG | |
| | | AGTVTSVEQCRKAVESGAEFIVSPHLDEEISQFCKEKGVFY | |
| | | MPGVMTPTELVKAMKLGHKVLKLFPGEVVGPQFVKAMK | |
| | | GPFPEVLFVPTGGVNLDNVCEWFKAGVIAVGVGSALVKGT | |
| | | PDEVREKAKAFVEKISACGVDSMAILSKVKGT | |
| CompA.077 | None | MGSSHHHHHHGSGSGNEEMEELFAKHKIVAVLRANSVEE | 135 |
| | | AIEKAVAVFAGGVHLIEITFTVPDADTVIKALSVLKEKGAII | |
| | | GAGTVTSVEQCRKAVESGAEFIVSPHLDEEISQFCKEKGVF | |
| | | YMPGVMTPTELVKAMKLGHKVLKLFPGEVVGPQFVKAM | |
| | | KGPFPEVLFVPTGGVNLDNVCEWFKAGVIAVGVGSALVK | |
| | | GTPDEVREKAKAFVEKISACGVDSMAILEKVKGT | |
| CompA.078 | None | MGSSHHHHHHGSGSEADLKMLKLFYQHRIVAVLRANSVE | 136 |
| | | EAIEKAVAVFAGGVHLIEITFTVPDADTVIKALSVLKEKGAI | |
| | | IGAGTVTSVEQCRKAVESGAEFIVSPHLDEEISQFCKEKGVF | |
| | | YMPGVMTPTELVKAMKLGHKILKLFPGEVVGPQFVKAMK | |
| | | GPFPEVAFVPTGGVNLDNVCEWIEAGAVAVGVGSALVKG | |
| | | TPDEVREKAKAFVEKANLCAGKYKKQLDKQLALQLGT | |
| CompA.079 | None | MGSSHHHHHHGSGSEADLKMLKLFYQHRIVAVLRANSVE | 137 |
| | | EAIEKAVAVFAGGVHLIEITFTVPDADTVIKALSVLKEKGAI | |
| | | IGAGTVTSVEQCRKAVESGAEFIVSPHLDEEISQFCKEKGVF | |
| | | YMPGVMTPTELVKAMKLGHKILKLFPGEVVGPQFVKAMK | |
| | | GPFPEVAFVPTGGVNLDNVCEWFEAGVVAVGVGSALVKG | |
| | | TPDEVREKAKAFVEKINLCAGKYKKQLDKQLALQLGT | |
| CompA.082 | Permutation | MGSSHHHHHHGSGGHKILKLFPGEVVGPQFVKAMKGPFP | 138 |
| | 3 | DVAFVPTGGVNLDNVCEWFKAGVLAVGVGSALVKGTPDE | |
| | (128-201:1-127) | VREKAKAFVEKINRCSEEAEKESEKIIKLETDPAALRMAKL | |
| | | FGKHKIVAVLRANSVEEAIEKAVAVFAGGVHLIEITFTVPD | |
| | | ADTVIKALSVLKEKGAIIGAGTVTSVEQCRKAVESGAEFIV | |
| | | SPHLDEEISQFCKEKGVFYMPGVMTPTELVKAMKLGSGSG | |
| | | T | |
| CompA.083 | None | MGSSHHHHHHGSGSEADLKMLKKFYTHKIVAVLRANSVE | 139 |
| | | EAIEKAVAVFAGGVHLIEITFTVPDADTVIKALSVLKEKGAI | |
| | | IGAGTVTSVEQCRKAVESGAEFIVSPHLDEEISQFCKEKGVP | |
| | | YMPGVMTPTELVKAMKLGHKLLKLFPGEVVGPQFVKAM | |
| | | KGPFPEVAFVPTGGVNLDNVCEWIEAGVVAVGVGSALVK | |
| | | GTPDEVREKAKAFVQKANECAGKYSDQLDKQLKLQLGT | |
| CompA.085 | None | MGSSHHHHHHGSGSEADLKMLKKFYTHKIVAVLRANSVE | 140 |
| | | EAIEKAVAVFAGGVHLIEITFTVPDADTVIKALSVLKEKGAI | |
| | | IGAGTVTSVEQCRKAVESGAEFIVSPHLDEEISQFCKEKGVP | |
| | | YMPGVMTPTELVKAMKLGHKLLKLFPGEVVGPQFVKAM | |
| | | KGPFPEVAFVPTGGVNLDNVCEWIEAGVVAVGVGSALVK | |
| | | GTPDEVREKAKAFVQKANECAGKYSSQLDKQLSLQLGT | |
| CompA.101 | None | MGSSHHHHHHGSTEAQALMLKLFVEHKIVAVLRANSVEE | 141 |
| | | AIEKAVAVFAGGVHLIEITFTVPDADTVIKALSVLKEKGAII | |
| | | GAGTVTSVEQCRKAVESGAEFIVSPHLDEEISQFCKEKGVF | |
| | | YMPGVMTPTELVKAMKLGHLVLKLFPGEVVGPQFVKAM | |
| | | KGPFPDVFFVPTGGVNLDNVCEWFKAGVLAVGVGSALVK | |
| | | GTPDEVREKAKAFVDKVTACIDAEVNAVHKKALAVAPKG | |
| | | T | |
| CompA.117 | None | MGSSHHHHHHGSGNPKEMIELFASHKIVAVLRANSVEEAIE | 142 |
| | | KAVAVFAGGVHLIEITFTVPDADTVIKALSVLKEKGAIIGA | |
| | | GTVTSVEQCRKAVESGAEFIVSPHLDEEISQFCKEKGVFYM | |
| | | PGVMTPTELVKAMKLGHKVLKLFPGEVVGPQFVKAMKGP | |
| | | FPEVLFVPTGGVNLDNVCEWFKAGVLAVGVGSALVKGTP | |
| | | DEVREKAKAFVEKISACGVNSMELVKKAEGT | |
| CompA.118 | None | MGSSHHHHHHGSGSGNKEMGELFATHKIVAVLRANSVEE | 143 |
| | | AIEKAVAVFAGGVHLIEITFTVPDADTVIKALSVLKEKGAII | |
| | | GAGTVTSVEQCRKAVESGAEFIVSPHLDEEISQFCKEKGVF | |
| | | YMPGVMTPTELVKAMKLGHKILKLFPGEVVGPQFVKAMK | |
| | | GPFPEVLFVPTGGVNLDNVCEWLEAGVLAVGVGSALVKG | |
| | | TPDEVREKAKAFVEKVKACPFDSMELFKKAEGT | |
| CompA.119 | None | MGSSHHHHHHGSGSGNKEMGELFASHKIVAVLRANSVEE | 144 |
| | | AIEKAVAVFAGGVHLIEITFTVPDADTVIKALSVLKEKGAII | |
| | | GAGTVTSVEQCRKAVESGAEFIVSPHLDEEISQFCKEKGVF | |
| | | YMPGVMTPTELVKAMKLGHKILKLFPGEVVGPQFVKAMK | |
| | | GPFPEVLFVPTGGVNLDNVCEWLEAGVLAVGVGSALVKG | |
| | | TPDEVREKAKAFVEKVSACPFDSMELFKKAEGT | |
| CompA.120 | None | MGSSHHHHHHGSGSGNKEMGELFATHKIVAVLRANSVEE | 145 |
| | | AIEKAVAVFAGGVHLIEITFTVPDADTVIKALSVLKEKGAII | |
| | | GAGTVTSVEQCRKAVESGAEFIVSPHLDEEISQFCKEKGVF | |
| | | YMPGVMTPTELVKAMKLGHKILKLFPGEVVGPQFVKAMK | |
| | | GPFPEVLFVPTGGVNLDNVCEWFEAGVLAVGVGSALVKG | |
| | | TPDEVREKAKAFVEKIKACPFDSMELFKKAEGT | |
| CompA.121 | None | MGSSHHHHHHGSGSGNKEMGELFASHKIVAVLRANSVEE | 146 |
| | | AIEKAVAVFAGGVHLIEITFTVPDADTVIKALSVLKEKGAII | |
| | | GAGTVTSVEQCRKAVESGAEFIVSPHLDEEISQFCKEKGVF | |
| | | YMPGVMTPTELVKAMKLGHKILKLFPGEVVGPQFVKAMK | |
| | | GPFPEVLFVPTGGVNLDNVCEWFEAGVLAVGVGSALVKG | |
| | | TPDEVREKAKAFVEKISACPFDSMELFKKAEGT | |
| CompA.123 | None | MGSSHHHHHHGSGSEADLKMLKKLYQERIVAVLRANSVE | 147 |
| | | EAIEKAVAVFAGGVKIIEITFTVPDADTVIKALSVLKEKGAII | |
| | | GAGTVTSVEQCRKAVESGAEFIVSPHLDEEISQFCKEKGVP | |
| | | YMPGVMTPTELVKAMKLGHKLLKLFPGEVVGPQFVKAM | |
| | | KGPFPEVAFVPTGGVNLDNVCEWIEAGVVAVGVGSALVK | |
| | | GTPDEVREKAKAFVEKANLCAGKYKKQLDKQLALQLGT | |
| CompA.124 | None | MGSSHHHHHHGSGSEADLKMLKKLYTEKIVAVLRANSVE | 148 |
| | | EAIEKAVAVFAGGVKIIEITFTVPDADTVIKALSVLKEKGAII | |
| | | GAGTVTSVEQCRKAVESGAEFIVSPHLDEEISQFCKEKGVP | |
| | | YMPGVMTPTELVKAMKLGHKLLKLFPGEVVGPQFVKAM | |
| | | KGPFPEVAFVPTGGVNLDNVCEWIEAGVVAVGVGSALVK | |
| | | GTPDEVREKAKAFVQKANECAGKYSDQLDKQLKLQLGT | |
| CompA.135 | None | MGSSHHHHHHGSGSGNEEIEEKFASEKIVAVLRANSVEEAI | 149 |
| | | EKAVAVFAGGVGIIEITFTVPDADTVIKALSVLKEKGASIGA | |
| | | GTVTSVEQCRKAVESGAEFIVSPHLDEEISQFCKEKGVPYM | |
| | | PGVMTPTELVKAMKLGHKVLKLFPGEVVGPQFVKAMKGP | |
| | | FPEVLFVPTGGVNLDNVCEWLKAGVIAVGVGSALVKGTPD | |
| | | EVREKAKAFVEKVSACGVDSMAILSKVKGT | |
| CompA.137 | None | MGSSHHHHHHGSGNVEIIEKFAKEKIVAVLRANSVEEAIEK | 150 |
| | | AVAVFAGGVGIIEITFTVPDADTVIKALSVLKEKGASIGAGT | |
| | | VTSVEQCRKAVESGAEFIVSPHLDEEISQFCKEKGVPYMPG | |
| | | VMTPTELVKAMKLGHKVLKLFPGEVVGPQFVKAMKGPFP | |
| | | DVLFVPTGGVNLDNVAEWLKAGVLAVGVGSALVKGTPDE | |
| | | VREKAKAFVEKVNASGVNAMKLVEKALGT | |
| CompA.138 | None | MGSSHHHHHHGSGNVEIIEKFASEKIVAVLRANSVEEAIEK | 151 |
| | | AVAVFAGGVGIIEITFTVPDADTVIKALSVLKEKGASIGAGT | |
| | | VTSVEQCRKAVESGAEFIVSPHLDEEISQFCKEKGVPYMPG | |
| | | VMTPTELVKAMKLGHKVLKLFPGEVVGPQFVKAMKGPFP | |
| | | DVLFVPTGGVNLDNVAEWLKAGVLAVGVGSALVKGTPDE | |
| | | VREKAKAFVEKVSASGVNSMELVKKALGT | |
| CompA.139 | None | MGSSHHHHHHGSGNVEIIEKFASEKIVAVLRANSVEEAIEK | 152 |
| | | AVAVFAGGVGIIEITFTVPDADTVIKALSVLKEKGASIGAGT | |
| | | VTSVEQCRKAVESGAEFIVSPHLDEEISQFCKEKGVPYMPG | |
| | | VMTPTELVKAMKLGHKVLKLFPGEVVGPQFVKAMKGPFP | |
| | | DVLFVPTGGVNLDNVAEWLKAGVLAVGVGSALVKGTPDE | |
| | | VREKAKAFVEKVSASGVNAMELVKKALGT | |
| CompA.142 | None | MGSSHHHHHHGSGNPKEIIEKFASEKIVAVLRANSVEEAIE | 153 |
| | | KAVAVFAGGVGIIEITFTVPDADTVIKALSVLKEKGASIGAG | |
| | | TVTSVEQCRKAVESGAEFIVSPHLDEEISQFCKEKGVPYMP | |
| | | GVMTPTELVKAMKLGHKVLKLFPGEVVGPQFVKAMKGPF | |
| | | PEVLFVPTGGVNLDNVCEWFKAGVLAVGVGSALVKGTPD | |
| | | EVREKAKAFVEKVSACGVNQMELVKKAEGT | |
| CompA.143 | None | MGSSHHHHHHGSGNPKEIIEKFASEKIVAVLRANSVEEAIE | 154 |
| | | KAVAVFAGGVGIIEITFTVPDADTVIKALSVLKEKGASIGAG | |
| | | TVTSVEQCRKAVESGAEFIVSPHLDEEISQFCKEKGVPYMP | |
| | | GVMTPTELVKAMKLGHKVLKLFPGEVVGPQFVKAMKGPF | |
| | | PEVLFVPTGGVNLDNVCEWFKAGVLAVGVGSALVKGTPD | |
| | | EVREKAKAFVEKVSACGVNSMELVKKAEGT | |
| CompA.144 | None | MGSSHHHHHHGSGSGNKEIGEKFAKEKIVAVLRANSVEEA | 155 |
| | | IEKAVAVFAGGVGIIEITFTVPDADTVIKALSVLKEKGASIG | |
| | | AGTVTSVEQCRKAVESGAEFIVSPHLDEEISQFCKEKGVPY | |
| | | MPGVMTPTELVKAMKLGHKVLKLFPGEVVGPQFVKAMK | |
| | | GPFPEVLFVPTGGVNLDNVCEWLEAGVLAVGVGSALVKG | |
| | | TPDEVREKAKAFVEKVNACPFNSMELFRKAEGT | |
| CompA.145 | None | MGSSHHHHHHGSGSGNKEIGEKFATEKIVAVLRANSVEEAI | 156 |
| | | EKAVAVFAGGVGIIEITFTVPDADTVIKALSVLKEKGASIGA | |
| | | GTVTSVEQCRKAVESGAEFIVSPHLDEEISQFCKEKGVPYM | |
| | | PGVMTPTELVKAMKLGHKVLKLFPGEVVGPQFVKAMKGP | |
| | | FPEVLFVPTGGVNLDNVCEWLEAGVLAVGVGSALVKGTP | |
| | | DEVREKAKAFVEKVRRCPFDSQELFKKAEGT | |
| CompA.146 | None | MGSSHHHHHHGSGSGNKEIGEKFATEKIVAVLRANSVEEAI | 157 |
| | | EKAVAVFAGGVGIIEITFTVPDADTVIKALSVLKEKGASIGA | |
| | | GTVTSVEQCRKAVESGAEFIVSPHLDEEISQFCKEKGVPYM | |
| | | PGVMTPTELVKAMKLGHKVLKLFPGEVVGPQFVKAMKGP | |
| | | FPEVLFVPTGGVNLDNVCEWLEAGVLAVGVGSALVKGTP | |
| | | DEVREKAKAFVEKVKACPFDSMELFKKAEGT | |
| CompA.147 | None | MGSSHHHHHHGSGSGNKEIGEKFASEKIVAVLRANSVEEAI | 158 |
| | | EKAVAVFAGGVGIIEITFTVPDADTVIKALSVLKEKGASIGA | |
| | | GTVTSVEQCRKAVESGAEFIVSPHLDEEISQFCKEKGVPYM | |
| | | PGVMTPTELVKAMKLGHKVLKLFPGEVVGPQFVKAMKGP | |
| | | FPEVLFVPTGGVNLDNVCEWLEAGVLAVGVGSALVKGTP | |
| | | DEVREKAKAFVEKVSACPFDSMELFKKAEGT | |

### Enumerated Embodiments

Clause 1. A polypeptide for forming a nanostructure, comprising an assembly domain comprising a polypeptide sequence at least 50%, at least 60%, at least 70%, at least 75%, at least 80%, at least 85%, at least 90%, at least 95%, at least 96%, at least 97%, at least 98%, at least 99%, or 100% identical to a first polypeptide sequence in Table 2.

Clause 2. A protein nanostructure, comprising a first component, and optionally a second component, wherein the first component comprises a first polypeptide comprising a first assembly domain comprising a polypeptide sequence at least 50%, at least 60%, at least 70%, at least 75%, at least 80%, at least 85%, at least 90%, at least 95%, at least 96%, at least 97%, at least 98%, at least 99%, or 100% identical to a polypeptide sequence in Table 2.

Clause 3. The nanostructure of clause 2, wherein the first component is a trimeric component comprising three copies of the first polypeptide.

Clause 4. The nanostructure of clause 1 or clause 2, wherein the nanostructure comprises the second component and wherein the second component comprises a second assembly domain comprising a polypeptide sequence at least 50%, at least 60%, at least 70%, at least 75%, at least 80%, at least 85%, at least 90%, at least 95%, at least 96%, at least 97%, at least 98%, at least 99%, or 100% identical to:

(SEQ ID NO: 26)
NQHSHKDHETVRIAVVRARWHAEIVDACVSAFEAAMRDIGGDRFAVDVE
DVPGAYEIPLHARTLAETGRYGAVLGTAFVVNGGIYRHEFVASAVINGM
MNVQLNTGVPVLSAVLTPHNYDKSKAHTLLFLALFAVKGMEAARACVEI
LAAREKIAA;
or
(SEQ ID NO: 27)
NQHSHKDYETVRIAVVRARWHAEIVDACVSAFEAAMADIGGDRFAVDVF
DVPGAYEIPLHARTLAETGRYGAVLGTAFVVNGGIYRHEFVASAVIDGM
MNVQLSTGVPVLSAVLTPHRYRDSDAHTLLFLALFAVKGMEAARACVEI
LAAREKIAA.

Clause 5. The nanostructure of clause 4, wherein the second component is a pentamer comprising five copies of the second polypeptide.

Clause 6. The nanostructure of any one of clauses 2-5, wherein the nanostructure comprises 20 copies of the first component.

Clause 7. The nanostructure of any one of clauses 2-6, wherein the nanostructure further comprises 12 copies of the second component.

Clause 8. The nanostructure of any one of clause 2-7, wherein the C terminus of the first polypeptide is accessible on the surface of the nanostructure.

Clause 9. The nanostructure of any one of clauses 2-8, wherein the first polypeptide is a fusion protein comprising, in N- to C-terminal order, the first assembly domain, optionally a linker, and a heterologous polypeptide sequence, preferably an antigen.

Clause 10. The nanostructure of clause 9, wherein the antigen is an ectodomain of a surface protein of a pathogenic organism, optionally a virus, or an antigenic fragment thereof.

Clause 11. The nanostructure of clause 10, wherein the antigen is an OspA or antigenic fragment thereof, preferably an OspA of Borrelia burgdorferi sensu lato.

Clause 12. The nanostructure of clause 9, wherein the antigen is an ectodomain of viral glycoprotein, or an antigenic fragment thereof.

Clause 13. The nanostructure of clause 9, wherein the antigen is an ectodomain of bacterial protein, or an antigenic fragment thereof.

Clause 14. A method of generating an immune response to an antigen or to a pathogenic organism in a subject in need thereof, comprising administering to the subject the nanostructure of any of clauses 2-13.

Clause 15. A pharmaceutical composition comprising the nanostructure of any of clauses 2-13.

Clause 16. A vaccine comprising the nanostructure of any of clauses 2-13.

Clause 17. A polynucleotide encoding the nanostructure of any of clauses 2-13 or the polypeptide of clause 1.

Clause 18. A host cell suitable for expression of the nanostructure of any of clauses 2-13 or the polypeptide of clause 1; and/or comprising the polynucleotide of clause 17.

Clause 19. A method of making a polypeptide or nanostructure, comprising culturing the host cell of clause 18 under conditions suitable for expression of the polypeptide or nanostructure.

SEQUENCE TABLE

| | | SEQ ID |
|---|---|---|
| Description | Sequence | NO: |
| I53-50A | MEELFKKHKIVAVLRANSVEEAIEKAVAVFAGGVHLIEITFTVPDADTV | 1 |
| | IKALSVLKEKGAIIGAGTVTSVEQCRKAVESGAEFIVSPHLDEEISQFCK | |
| | EKGVFYMPGVMTPTELVKAMKLGHTILKLFPGEVVGPQFVKAMKGP | |
| | FPNVKFVPTGGVNLDNVCEWFKAGVLAVGVGSALVKGTPDEVREKA | |
| | KAFVEKIRGCTE | |
| N-terminal polypeptide | MKMEELFKKHKIVAVLRANSVEEAIEKAVAVFAGGVHLIEITFTVPDA | 2 |
| segment-A | DTVIKALSVLKEKGAIIGAGTVTSV | |
| N-terminal polypeptide | MKMEELFKKHKIVAVLRANSVEEAIEKAVAVFAGGVHLIEITFTVPDA | 3 |
| segment-B | DTVIKALSVLKEKGAIIGAGTVTSVEQCRKAVESGAEFIVSPHLDEEIS | |
| | QFCKEKGVF | |
| N-terminal polypeptide | MKMEELFKKHKIVAVLRANSVEEAIEKAVAVFAGGVHLIEITFTVPDA | 4 |
| segment-C | DTVIKALSVLKEKGAIIGAGTVTSVEQCRKAVESGAEFIVSPHLDEEIS | |
| | QFCKEKGVFYMPGVMTPTELVKAMKLGHDI | |
| C-terminal polypeptide | EQCRKAVESGAEFIVSPHLDEEISQFCKEKGVFYMPGVMTPTELVKAM | 5 |
| segment-A | KLGHDILKLFPGEVVGPQFVKAMKGPFPNVKFVPTGGVNLDNVCKW | |
| | FKAGVLAVGVGKALVKGKPDEVREKAKKFVKKIR | |
| C-terminal polypeptide | YMPGVMTPTELVKAMKLGHDILKLFPGEVVGPQFVKAMKGPFPNVK | 6 |
| segment-B | FVPTGGVNLDNVCKWFKAGVLAVGVGKALVKGKPDEVREKAKKFV | |
| | KKIR | |
| C-terminal polypeptide | LKLFPGEVVGPQFVKAMKGPFPNVKFVPTGGVNLDNVCKWFKAGVL | 7 |
| segment-C | AVGVGKALVKGKPDEVREKAKKFVKKIR | |
| linking polypeptide | KKVGEELIKLVTRPEDR | 8 |
| segment 1 | | |
| linking polypeptide | KEVGKKLLLLVTDPADKK | 9 |
| segment 2 | | |
| linking polypeptide | DKESEKLIKLETDPAMLRM | 10 |
| segment 3 | | |
| linking polypeptide | EKEGKKALKLETDPAMKKMV | 11 |
| segment 4 | | |
| linking polypeptide | VQAVHKKALAVAPKLTEAQALM | 12 |
| segment 5 | | |
| linking polypeptide | ENKKVGEELIKLVTRPED | 13 |
| segment 6 | | |
| linking polypeptide | VNNEVGKKLLLLVTDPAD | 14 |
| segment 7 | | |
| linking polypeptide | ENEKEGKKALKLETDPAM | 15 |
| segment 8 | | |
| linking polypeptide | EAKKESEKLIKLETDPAM | 16 |
| segment 9 | | |
| linking polypeptide | PEVEAVHEKALAVAPKLTEAQ | 17 |
| segment 10 | | |
| linking polypeptide | ENKKVGEELIKLVTRPED | 18 |
| segment 11 | | |
| linking polypeptide | ENEKEGKKALKLETDPAM | 19 |
| segment 12 | | |
| linking polypeptide | EAKKESEKLIKLETDPAM | 20 |
| segment 13 | | |
| linking polypeptide | PEVEAVHEKALAVAPKLTEAQ | 21 |
| segment 14 | | |
| assembly domain 1 | XXXXXVEQCRKAVESGAEFIVSPHLDEEISQFCKEKGVFYMPGVMTP | 22 |
| | TELVKAMKLGHDILKLFPGEVVGPQFVKAMKGPFPNVKFVPTGGVNL | |
| | DNVCKWFKAGVLAVGVGKALVKGKPDEVREKAKKFVKKIRGCTEG | |
| | TXXXXXXXXXXXXXXXXMKMEELFKKHKIVAVLRANSVEEAIEKAV | |
| | AVFAGGVHLIEITFTVPDADTVIKALSVLKEKGAIIGAGTVTS | |
| assembly domain 2 | XXXXXXYMPGVMTPTELVKAMKLGHDILKLFPGEVVGPQFVKAMK | 23 |
| | GPFPNVKFVPTGGVNLDNVCKWFKAGVLAVGVGKALVKGKPDEVRE | |
| | KAKKFVKKIRGCTEXXXXXXXXXXXXXXXXXXMKMEELFKKHKIV | |
| | AVLRANSVEEAIEKAVAVFAGGVHLIEITFTVPDADTVIKALSVLKEKG | |
| | AIIGAGTVTSVEQCRKAVESGAEFIVSPHLDEEISQFCKEKGVF | |
| assembly domain 3 | XXXXXXXXLKLFPGEVVGPQFVKAMKGPFPNVKFVPTGGVNLDNVC | 24 |
| | KWFKAGVLAVGVGKALVKGKPDEVREKAKKFVKKIRGCTEXXXXX | |
| | XXXXXXXXXXXXXMKMEELFKKHKIVAVLRANSVEEAIEKAVAVFA | |
| | GGVHLIEITFTVPDADTVIKALSVLKEKGAIIGAGTVTSVEQCRKAVES | |
| | GAEFIVSPHLDEEISQFCKEKGVFYMPGVMTPTELVKAMKLGHDI | |
| assembly domain 4 | XXXXXXXMKMEELFKKHKIVAVLRANSVEEAIEKAVAVFAGGVHLIEI | 25 |
| | TFTVPDADTVIKALSVLKEKGAIIGAGTVTSVEQCRKAVESGAEFIVSP | |
| | HLDEEISQFCKEKGVFYMPGVMTPTELVKAMKLGHDILKLFPGEVVG | |
| | PQFVKAMKGPFPNVKFVPTGGVNLDNVCKWFKAGVLAVGVGKALV | |
| | KGKPDEVREKAKKFVKKIRGCTEXXXXXXXXXXXXXXXX | |
| I53-50B.4PosT1 | NQHSHKDHETVRIAVVRARWHAEIVDACVSAFEAAMRDIGGDRFAV | 26 |
| | DVFDVPGAYEIPLHARTLAETGRYGAVLGTAFVVNGGIYRHEFVASAV | |
| | INGMMNVQLNTGVPVLSAVLTPHNYDKSKAHTLLFLALFAVKGMEA | |
| | ARACVEILAAREKIAA | |
| I53-50B | NQHSHKDYETVRIAVVRARWHAEIVDACVSAFEAAMADIGGDRFAV | 27 |
| | DVFDVPGAYEIPLHARTLAETGRYGAVLGTAFVVNGGIYRHEFVASAV | |
| | IDGMMNVQLSTGVPVLSAVLTPHRYRDSDAHTLLFLALFAVKGMEAA | |
| | RACVEILAAREKIAA | |
| OspA of Borrelia | MKKYLLGIGLILALIACKQNVSSLDEKNSVSVDVPGGMKVLVSKEKN | 28 |
| burgdorferi sensu lato | KDGKYDLMATVDNVDLKGTSDKNNGSGILEGVKADKSKVKLTVADD | |
| | LSKTTLEVLKEDGTVVSRKVTSKDKSTTEAKFNEKGELSEKTMTRAN | |
| | GTTLEYSQMTNEDNAAKAVETLKNGIKFEGNLASGKTAVEIKEGTVTL | |
| | KREIDKNGKVTVSLNDTASGSKKTASWQESTSTLTISANSKKTKDLVF | |
| | LTNGTITVQNYDSAGTKLEGSAAEIKKLDELKNALR | |
| OspA of Borrelia | MKQYLLGFTLVFALIACAQKGANPEQKGGDADVLNTSKTEKYLNKN | 29 |
| burgdorferi sensu lato | LPSEAEDLVSLFNDSEIFVSKEKNKDGKYVLRAIVDTVELKGVADKND | |
| | GSEGKLEGLKPDNSKVTMSISKDQNTITIETRDSSNTKVASKVFKKDG | |
| | SLTEESYKAGQLDSKKLTRSNKTTLEYSDMTNAENATTAIETLKNGIEF | |
| | KGSLVGGKATLQIVESTVTLTREIDKDGKLKIYLKDTASSSKKTVSWN | |
| | DTDTLTISAEGKKTKDLVFLTDGTITVQNYDSASGTTLEGTATEIKNLE | |
| | ALKTALK | |
| fHbp of N. Meningitidis | MNRTAFCCLSLTTALILTACSSGGGGVAADIGAGLADALTAPLDHKDK | 30 |
| | GLQSLTLDQSVRKNEKLKLAAQGAEKTYGNGDSLNTGKLKNDKVSR | |
| | FDFIRQIEVDGQLITLESGEFQVYKQSHSALTAFQTEQIQDSEHSGKMV | |
| | AKRQFRIGDIAGEHTSFDKLPEGGRATYRGTAFGSDDAGGKLTYTIDF | |
| | AAKQGNGKIEHLKSPELNVDLAAADIKPDGKRHAVISGSVLYNQAEK | |
| | GSYSLGIFGGKAQEVAGSAEVKTVNGIRHIGLAAKQ | |
| RSV G protein | MSKNKDQRTAKTLERTWDTLNHLLFISSCLYKLNLKSVAQITLSILAMI | 31 |
| | ISTSLIIAAIIFIASANHKVTPTTAIIQDATSQIKNTTPTYLTQNPQLGISPS | |
| | NPSEITSQITTILASTTPGVKSTLQSTTVKTKNTTTTQTQPSKPTTKQRQ | |
| | NKPPSKPNNDFHFEVFNFVPCSICSNNPTCWAICKRIPNKKPGKKTTTK | |
| | PTKKPTLKTTKKDPKPQTTKSKEVPTTKPTEEPTINTTKTNIITTLLTSN | |
| | TTGNPELTSQMETFHSTSSEGNPSPSQVSTTSEYPSQPSSPPNTPRQ | |
| hMPV G protein | MEVKVENIRAIDMLKARVKNRVARSKCFKNASLILIGITTLSIALNIYLII | 32 |
| | NYTIQKTSSESEHHTSSPPTESNKEASTISTDNPDINPNSQHPTQQSTEN | |
| | PTLNPAASVSPSETEPASTPDTTNRLSSVDRSTAQPSESRTKTKPTVHTR | |
| | NNPSTASSTQSPPRATTKAIRRATTFRMSSTGKRPTTTSVQSDSSTTTQ | |
| | NHEETGSANPQASVSTMQN | |
| CompA.005 | ENHARFAALRAELAGT | 33 |
| CompA.006 | ENPELTKEVAAFLAGT | 34 |
| CompA.009 | EYSEQFEARKKKLEGT | 35 |
| CompA.010 | RIDEEYQKRLEKLRGT | 36 |
| CompA.011 | KYKEQLDKQLKLQLGT | 37 |
| CompA.012 | KEKEYFEEQLEKLKGT | 38 |
| CompA.013 | YSKARLAEIKKALAGT | 39 |
| CompA.014 | AADEHMAAIMAALKGT | 40 |
| CompA.015 | VGDKLLAELKAQLAGT | 41 |
| CompA.016 | YLKANAEKLHKLLAGT | 42 |
| CompA.017 | ILAKLKAKILAKLKGT | 43 |
| CompA.018 | YSQATLKEILKALAGT | 44 |
| CompA.025 | FSKEACKKAILETKDGSGSGT | 45 |
| CompA.029 | PEVQAVHKKALAVAPKGT | 46 |
| CompA.030 | EEVKAVQQKALALAPKLTGSGSGT | 47 |
| CompA.031 | AEVAANQAKALSLAPPEAGGT | 48 |
| CompA.032 | AEVEANQAKALSLAPAPAGSGSGT | 49 |
| CompA.033 | EEVEAVQKKALSIAEELEGSGSGT | 50 |
| CompA.034 | EEVAAVQKKALSLAPKEPSGT | 51 |
| CompA.035 | DTLALLKERKGT | 52 |
| CompA.036 | DSMAILEKVKGT | 53 |
| CompA.037 | NQMELVKKVFGT | 54 |
| CompA.038 | NAMELVKKALGT | 55 |
| CompA.039 | NQMELVKKAEGT | 56 |
| CompA.040 | DSMELFKKAEGT | 57 |
| CompA.041 | DAMELLKEAEAIMGT | 58 |
| CompA.042 | DPMKLVEEVEKLLGT | 59 |
| CompA.043 | DPMAEVEKAKALEGT | 60 |
| CompA.044 | DPMALVDKVLALFGT | 61 |
| CompA.001 (I53-50A) | MKMEELFKKHKIVAVLRANSVEEAIEKAVAVFAGGVHLIEITFTVPDA | 62 |
| | DTVIKALSVLKEKGAIIGAGTVTSVEQCRKAVESGAEFIVSPHLDEEIS | |
| | QFCKEKGVFYMPGVMTPTELVKAMKLGHDILKLFPGEVVGPQFVKA | |
| | MKGPFPNVKFVPTGGVNLDNVCKWFKAGVLAVGVGKALVKGKPDE | |
| | VREKAKKFVKKIRGCTEGT | |
| CompA.002 | MGSGSVEQCRKAVEAGAEYIVSPHLDEEISQFCKEKGIPYMPGVMTPT | 63 |
| | ELVKAMKLGHLLLKLFPGEVVGPQFVKAMKKTFPKARFVPTGGVNL | |
| | DNVCEWFKAGVLAVGVGSALVKGTPDEVREKAKAFVEEAEKCSEEN | |
| | KKVGEELIKLVTRPEDREMVEIFYKEKIVAVLRANSVEEAIEKAVAVFA | |
| | GGVTIIEITFTVPDADTVIKALSVLKEKGAVIGAGTVTGSGT | |
| CompA.003 | MGSIPYMPGVMTPTELVKAMKLGHLLLKLFPGEVVGPQFVKAMKKT | 64 |
| | FPKARFVPTGGVNLDNVCEWFKAGVLAVGVGSALVKGTPDEVREKA | |
| | KAFVEEAAKCSEENKKVGEELIKLVTRPEDREMVEIFYEKKIVAVLRA | |
| | NSVEEAIEKAVAVFAGGVTIIEITFTVPDADTVIKALSVLKEKGAVIGAG | |
| | TVTSVEQCRKAVEAGAEYIVSPHLDEEISQFCKEKGSGSGT | |
| CompA.004 | MGSGHLLLKLFPGEVVGPQFVKAMKKTFPKARFVPTGGVNLDNVCE | 65 |
| | WFKAGVLAVGVGSALVKGTPDEVREKAKAFVEEAAKCSEENKKVGE | |
| | ELIKLVTRPEDREMVEIFYKEKIVAVLRANSVEEAIEKAVAVFAGGVTII | |
| | EITFTVPDADTVIKALSVLKEKGAVIGAGTVTSVEQCRKAVEAGAEYI | |
| | VSPHLDEEISQFCKEKGIPYMPGVMTPTELVKAMKLGSGSGT | |
| CompA.005 | MGAPADRELLRKLLENRIVAVLRANSVEEAIEKAVAVFAGGVTIIEITFT | 66 |
| | VPDADTVIKALSVLKEKGAVIGAGTVTSVEQCRKAVEAGAEYIVSPHL | |
| | DEEISQFCKEKGIPYMPGVMTPTELVKAMKLGHLLLKLFPGEVVGPQF | |
| | VKAMKKTYPEARFVPTGGVNLDNVCEWIKAGAIAVGVGSALVKGTP | |
| | DEVREKAKAFVEEAAKCAAENHARFAALRAELAGT | |
| CompA.006 | MGAPEEKKMIALLAENPIVAVLRANSVEEAIEKAVAVFAGGVTIIEITFT | 67 |
| | VPDADTVIKALSVLKEKGAVIGAGTVTSVEQCRKAVEAGAEYIVSPHL | |
| | DEEISQFCKEKGIPYMPGVMTPTELVKAMKLGHLLLKLFPGEVVGPQF | |
| | VKAMKKTYPEARFVPTGGVNLDNVCEWIKAGALAVGVGSALVKGTP | |
| | DEVREKAKAFVEEALKCRGENPELTKEVAAFLAGT | |
| CompA.007 | MGSGSVEQCRKAVEAGAEYIVSPHLDEEISQFCKEKGVPYMPGVMTP | 68 |
| | TELVKAMKLGHLLLKLFPGEVVGPQFVKAMKKTFPDAAFVPTGGVN | |
| | LDNVCEWFKAGVLAVGVGSALVKGTPDEVREKAKAFVEKAKKCDE | |
| | VNKEVGKKLLLLVTDPADKKMVERFYKEKIVAVLRANSVEEAIEKAV | |
| | AVFAGGVTIIEITFTVPDADTVIKALSVLKEKGAIIGAGTVTGSGT | |
| CompA.008 | MGSGVPYMPGVMTPTELVKAMKLGHLLLKLFPGEVVGPQFVKAMK | 69 |
| | KTFPDAAFVPTGGVNLDNVCEWFKAGVLAVGVGSALVKGTPDEVRE | |
| | KAKAFVEKAKKCDQVNNEVGKKLLLLVTDPADKKMVERFYEEKIVA | |
| | VLRANSVEEAIEKAVAVFAGGVTIIEITFTVPDADTVIKALSVLKEKGAI | |
| | IGAGTVTSVEQCRKAVEAGAEYIVSPHLDEEISQFCKEKGSGSGT | |
| CompA.009 | MGDPKELAMLKAFLEEKIVAVLRANSVEEAIEKAVAVFAGGVKIIEITF | 70 |
| | TVPDADTVIKALSVLKEKGAIIGAGTVTSVEQCRKAVEAGAEYIVSPH | |
| | LDEEISQFCKEKGVPYMPGVMTPTELVKAMKLGHQLLKLFPGEVVGP | |
| | QFVKAMKKTYPDAAFVPTGGVNLDNVCEWFKAGATAVGVGSALVK | |
| | GTPDEVREKAKAFVEKAAKCEGEYSEQFEARKKKLEGT | |
| CompA.010 | MPEKEREIMIAFLKNRIVAVLRANSVEEAIEKAVAVFAGGVKIIEITFTV | 71 |
| | PDADTVIKALSVLKEKGAIIGAGTVTSVEQCRKAVEAGAEYIVSPHLD | |
| | EEISQFCKEKGVPYMPGVMTPTELVKAMKLGHRLLKLFPGEVVGPQF | |
| | VKAMKKTYPDAAFVPTGGVNLDNVCEWFDAGAVAVGVGSALVKGT | |
| | PDEVREKAKAFVEKAAKCRARIDEEYQKRLEKLRGT | |
| CompA.011 | MGSEADLKMLKKLYEEKIVAVLRANSVEEAIEKAVAVFAGGVKIIEITF | 72 |
| | TVPDADTVIKALSVLKEKGAIIGAGTVTSVEQCRKAVEAGAEYIVSPH | |
| | LDEEISQFCKEKGVPYMPGVMTPTELVKAMKLGHKLLKLFPGEVVGP | |
| | QFVKAMKKTYPEAAFVPTGGVNLDNVCEWIEAGAVAVGVGSALVKG | |
| | TPDEVREKAKAFVEKANECAGKYKEQLDKQLKLQLGT | |
| CompA.012 | MGLPEVELKMIEKIMEEGIVAVLRANSVEEAIEKAVAVFAGGVKIIEITF | 73 |
| | TVPDADTVIKALSVLKEKGAIIGAGTVTSVEQCRKAVEAGAEYIVSPH | |
| | LDEEISQFCKEKGVPYMPGVMTPTELVKAMKLGHVLLKLFPGEVVGP | |
| | QFVKAMKKTYPNVAFVPTGGVNLDNVCEWIEAGAAAVGVGSALVKG | |
| | TPDEVREKAKAFVEKANECRAKEKEYFEEQLEKLKGT | |
| CompA.013 | MGVDEKDLKLLEALAANRIVAVLRANSVEEAIEKAVAVFAGGVTIIEIT | 74 |
| | FTVPDADTVIKALSVLKEKGAIIGAGTVTSVEQCRKAVEAGAEYIVSP | |
| | HLDEEISQFCKEKGVPYMPGVMTPTELVKAMKLGHLLLKLFPGEVVG | |
| | PQFVKAMKKTYPTAAFVPTGGVNLDNVCEWLKAGAVAVGVGSALVK | |
| | GTPDEVREKAKAFVAKADEYAKYSKARLAEIKKALAGT | |
| CompA.014 | MGVSEKEIEMLKKFNEARIVAVLRANSVEEAIEKAVAVFAGGVTIIEITF | 75 |
| | TVPDADTVIKALSVLKEKGAIIGAGTVTSVEQCRKAVEAGAEYIVSPH | |
| | LDEEISQFCKEKGVPYMPGVMTPTELVKAMKLGHLLLKLFPGEVVGP | |
| | QFVKAMKKTYPLAAFVPTGGVNLDNVCEWLEAGCIAVGVGSALVKG | |
| | TPDEVREKAKAFVAKARECAAAADEHMAAIMAALKGT | |
| CompA.015 | MGLSPAEQAMLLAVVENRIVAVLRANSVEEAIEKAVAVFAGGVTIIEITF | 76 |
| | TVPDADTVIKALSVLKEKGAIIGAGTVTSVEQCRKAVEAGAEYIVSPH | |
| | LDEEISQFCKEKGVPYMPGVMTPTELVKAMKLGHLLLKLFPGEVVGP | |
| | QFVKAMKKTYPGVAFVPTGGVNLDNVCEWLEAGAAAVGVGSALVK | |
| | GTPDEVREKAKAFVAKADEMGAVGDKLLAELKAQLAGT | |
| CompA.016 | MGYPEAQIELLDKVIKEGIVAVLRANSVEEAIEKAVAVFAGGVTIIEITF | 77 |
| | TVPDADTVIKALSVLKEKGAIIGAGTVTSVEQCRKAVEAGAEYIVSPH | |
| | LDEEISQFCKEKGVPYMPGVMTPTELVKAMKLGHLLLKLFPGEVVGP | |
| | QFVKAMKKTYPGAAFVPTGGVNLDNVCEWLKAGAAAVGVGSALVK | |
| | GTPDEVREKAKAFVAKAKECSKYLKANAEKLHKLLAGT | |
| CompA.017 | MGLSEKEIAIIEAFLENPIVAVLRANSVEEAIEKAVAVFAGGVTIIEITFTV | 78 |
| | PDADTVIKALSVLKEKGAIIGAGTVTSVEQCRKAVEAGAEYIVSPHLD | |
| | EEISQFCKEKGVPYMPGVMTPTELVKAMKLGHLLLKLFPGEVVGPQF | |
| | VKAMKKTYPDVAFVPTGGVNLDNVCEWLEAGAVAVGVGSALVKGTP | |
| | DEVREKAKAFVAKAGEKAAILAKLKAKILAKLKGT | |
| CompA.018 | MGVSEADLALLKALAENQIVAVLRANSVEEAIEKAVAVFAGGVTIIEIT | 79 |
| | FTVPDADTVIKALSVLKEKGAIIGAGTVTSVEQCRKAVEAGAEYIVSP | |
| | HLDEEISQFCKEKGVPYMPGVMTPTELVKAMKLGHLLLKLFPGEVVG | |
| | PQFVKAMKKTYPSAAFVPTGGVNLDNVCEWLKAGCIAVGVGSALVK | |
| | GTPDEVREKAKAFVAKADECAKYSQATLKEILKALAGT | |
| CompA.019 | MGSGSVEQCRKAVEAGAEYIVSPHLDEEISQFCKEKGIPYMPGVMTPT | 80 |
| | ELVKAMKLGHRVLKLFPGEVVGPQFVKAMKKTFPDARFVPTGGVNL | |
| | DNVCEWFKAGVLAVGVGSALVKGTPDEVREKAKAFVEKAAECSEEN | |
| | EKEGKKALKLETDPAMKKMVKIFYKEKIVAVLRANSVEEAIEKAVAVF | |
| | AGGVTVIEITFTVPDADTVIKALSVLKEKGAVIGAGTVTGSGT | |
| CompA.020 | MGSGIPYMPGVMTPTELVKAMKLGHRVLKLFPGEVVGPQFVKAMKK | 81 |
| | TFPDARFVPTGGVNLDNVCEWFKAGVLAVGVGSALVKGTPDEVREK | |
| | AKAFVEKAAECSEENEKEGKKALKLETDPAMKKMVKIFYKEKIVAVL | |
| | RANSVEEAIEKAVAVFAGGVTVIEITFTVPDADTVIKALSVLKEKGAVI | |
| | GAGTVTSVEQCRKAVEAGAEYIVSPHLDEEISQFCKEKGSGSGT | |
| CompA.021 | MGSGGHRVLKLFPGEVVGPQFVKAMKKTFPDARFVPTGGVNLDNVC | 82 |
| | EWFKAGVLAVGVGSALVKGTPDEVREKAKAFVEKAAECSEENEKEG | |
| | KKALKLETDPAMKKMVKIFYKEKIVAVLRANSVEEAIEKAVAVFAGGV | |
| | TVIEITFTVPDADTVIKALSVLKEKGAVIGAGTVTSVEQCRKAVEAGA | |
| | EYIVSPHLDEEISQFCKEKGIPYMPGVMTPTELVKAMKLGSGSGT | |
| CompA.022 | MGSGSVEQCRKAVEAGAEYIVSPHLDEEISQFCKEKGIPYMPGVMTPT | 83 |
| | ELVKAMKLGHKLLKLFPGEVVGPQFVKAMKKTFPDAAFVPTGGVNL | |
| | DNVCEWFKAGVLAVGVGSALVKGTPDEVREKAKAFVEKAKECSEEA | |
| | DKESEKLIKLETDPAMLRMAKIFGKEKIVAVLRANSVEEAIEKAVAVFA | |
| | GGVTVIEITFTVPDADTVIKALSVLKEKGAVIGAGTVTGSGT | |
| CompA.023 | MGSGIPYMPGVMTPTELVKAMKLGHKLLKLFPGEVVGPQFVKAMKK | 84 |
| | TFPDAAFVPTGGVNLDNVCEWFKAGVLAVGVGSALVKGTPDEVREK | |
| | AKAFVEKAEECSSEAKKESEKLIKLETDPAMLRMAKIFGKEKIVAVLR | |
| | ANSVEEAIEKAVAVFAGGVTVIEITFTVPDADTVIKALSVLKEKGAVIG | |
| | AGTVTSVEQCRKAVEAGAEYIVSPHLDEEISQFCKEKGSGSGT | |
| CompA.024 | MGSGGHKLLKLFPGEVVGPQFVKAMKKTFPDAAFVPTGGVNLDNVC | 85 |
| | EWFKAGVLAVGVGSALVKGTPDEVREKAKAFVEKAEECSEEAKKES | |
| | EKLIKLETDPAMLRMAKIFGKEKIVAVLRANSVEEAIEKAVAVFAGGVT | |
| | VIEITFTVPDADTVIKALSVLKEKGAVIGAGTVTSVEQCRKAVEAGAE | |
| | YIVSPHLDEEISQFCKEKGIPYMPGVMTPTELVKAMKLGSGSGT | |
| CompA.025 | MGDKAMASMAKQFCKNKIVAVLRANSVEEAIEKAVAVFAGGVAIIEIT | 86 |
| | FTVPDADTVIKALSVLKEKGASIGAGTVTSVEQCRKAVEAGADYIVSP | |
| | HLDEEISQFCKEKGVPYMPGVMTPTELVKAMKLGHVILKLFPGEVVG | |
| | PQFVKAMKKTFPNAQFVPTGGVNLDNVCEWFKAGVLAVGVGSALV | |
| | KGTPDEVREKAKAFVEKVKECAHFSKEACKKAILETKDGSGSGT | |
| CompA.026 | MGSGSVEQCRKAVEAGADYIVSPHLDEEISQFCKEKGVAYMPGVMTP | 87 |
| | TELVKAMKLGHLVLKLFPGEVVGPQFVKAMKKTFPDVFFVPTGGVN | |
| | LDNVCEWFKAGVLAVGVGSALVKGTPDEVREKAKAFVDKVIACYGP | |
| | EVQAVHKKALAVAPKLTEAQALMLKAFVEEKIVAVLRANSVEEAIEKA | |
| | VAVFAGGVNIIEITFTVPDADTVIKALSVLKEKGAIIGAGTVTGSGT | |
| CompA.027 | MGSGVAYMPGVMTPTELVKAMKLGHLVLKLFPGEVVGPQFVKAMK | 88 |
| | KTFPDVFFVPTGGVNLDNVCEWFKAGVLAVGVGSALVKGTPDEVRE | |
| | KAKAFVDKVTACYGPEVEAVHEKALAVAPKLTEAQALMLKAFVEEKI | |
| | VAVLRANSVEEAIEKAVAVFAGGVNIIEITFTVPDADTVIKALSVLKEK | |
| | GAIIGAGTVTSVEQCRKAVEAGADYIVSPHLDEEISQFCKEKGSGSGT | |
| CompA.028 | MGSSGHLVLKLFPGEVVGPQFVKAMKKTFPDVFFVPTGGVNLDNVC | 89 |
| | EWFKAGVLAVGVGSALVKGTPDEVREKAKAFVDKVTACYGPEVEAV | |
| | HEKALAVAPKLTEAQALMLKAFVEEKIVAVLRANSVEEAIEKAVAVFA | |
| | GGVNIIEITFTVPDADTVIKALSVLKEKGAIIGAGTVTSVEQCRKAVEA | |
| | GADYIVSPHLDEEISQFCKEVGVAYMPGVMTPTELVKAMKLGSGSGT | |
| CompA.029 | MTEAQALMLKAFVEEKIVAVLRANSVEEAIEKAVAVFAGGVNIIEITFT | 90 |
| | VPDADTVIKALSVLKEKGAIIGAGTVTSVEQCRKAVEAGADYIVSPHL | |
| | DEEISQFCKEVGVAYMPGVMTPTELVKAMKLGHLVLKLFPGEVVGPQ | |
| | FVKAMKKTFPDVFFVPTGGVNLDNVCEWFKAGVLAVGVGSALVKGT | |
| | PDEVREKAKAFVEKVLACIGPEVQAVHKKALAVAPKGT | |
| CompA.030 | MGTEANALMLKRFVEEKIVAVLRANSVEEAIEKAVAVFAGGVNIIEITF | 91 |
| | TVPDADTVIKALSVLKEKGAVIGAGTVTSVEQCRKAVEAGAAYIVSPH | |
| | LDEEISQFCKEVGVAYMPGVMTPTELVKAMKLGHLVLKLFPGEVVGP | |
| | QFVKAMKKTFPDAVFVPTGGVNLDNVCEWFKAGVLAVGVGSALVK | |
| | GTPDEVREKAKAFREKVAACDGEEVKAVQQKALALAPKLTGSGSGT | |
| CompA.031 | MTPAEALMLKRFVKEKIVAVLRANSVEEAIEKAVAVFAGGVNIIEITFT | 92 |
| | VPDADTVIKALSVLKEKGAIIGAGTVTSVEQCRKAVEAGADYIVSPHL | |
| | DEEISQFCKEVGVAYMPGVMTPTELVKAMKLGHIILKLFPGEVVGPQF | |
| | VKAMKKTFPDAVFVPTGGVNLDNVCEWFKAGVVAVGVGSALVKGTP | |
| | DEVREKAKAFREKVATCVGAEVAANQAKALSLAPPEAGGT | |
| CompA.032 | MTPAEALMLKRFVKEKIVAVLRANSVEEAIEKAVAVFAGGVNIIEITFT | 93 |
| | VPDADTVIKALSVLKEKGAIIGAGTVTSVEQCRKAVEAGADYIVSPHL | |
| | DEEISQFCKEVGVAYMPGVMTPTELVKAMKLGHIILKLFPGEVVGPQF | |
| | VKAMKKTFPDAVFVPTGGVNLDNVCEWFKAGVVAVGVGSALVKGTP | |
| | DEVREKAKAFREKVATCVGAEVEANQAKALSLAPAPAGSGSGT | |
| CompA.033 | MTPAEALMLKRFVEEKIVAVLRANSVEEAIEKAVAVFAGGVNIIEITFTV | 94 |
| | PDADTVIKALSVLKEKGAVIGAGTVTSVEQCRKAVEAGADYIVSPHLD | |
| | EEISQFCKEVGVAYMPGVMTPTELVKAMKLGHILLKLFPGEVVGPQF | |
| | VKAMKETFPDAFFVPTGGVNLDNVCEWFKAGVVAVGVGSALVKGTP | |
| | DEVREKAKAFVEKVNSCTGEEVEAVQKKALSIAEELEGSGSGT | |
| CompA.034 | MTPAEALMLKRFVEEKIVAVLRANSVEEAIEKAVAVFAGGVNIIEITFTV | 95 |
| | PDADTVIKALSVLKEKGAVIGAGTVTSVEQCRKAVEAGADYIVSPHLD | |
| | EEISQFCKEVGVAYMPGVMTPTELVKAMKLGHILLKLFPGEVVGPQF | |
| | VKAMKETFPDAFFVPTGGVNLDNVCEWFKAGVVAVGVGSALVKGTP | |
| | DEVREKAKAFKAKVASCTGEEVAAVQKKALSLAPKEPSGT | |
| CompA.035 | MGNKEEIEEKFAREKIVAVLRANSVEEAIEKAVAVFAGGVGIIEITFTVP | 96 |
| | DADTVIKALSVLKEKGASIGAGTVTSVEQCRKAVEAGAEYIVSPHLDE | |
| | EISQFCKELGIPYMPGVMTPTELVKAMKLGHKVLKLFPGEVVGPQFV | |
| | KAMKKTYPDVLFVPTGGVNLDNVCEWLKAGALAVGVGSALVKGTP | |
| | DEVREKAKAFVEKVKACGVDTLALLKERKGT | |
| CompA.036 | MGNEEIEEKFAKEKIVAVLRANSVEEAIEKAVAVFAGGVGIIEITFTVPD | 97 |
| | ADTVIKALSVLKEKGASIGAGTVTSVEQCRKAVEAGAEYIVSPHLDEE | |
| | ISQFCKELGIPYMPGVMTPTELVKAMKLGHKVLKLFPGEVVGPQFVK | |
| | AMKKTYPEALFVPTGGVNLDNVCEWLKAGAIAVGVGSALVKGTPDE | |
| | VREKAKAFVEKVKACGVDSMAILEKVKGT | |
| CompA.037 | MGNKEIIEKFAKEKIVAVLRANSVEEAIEKAVAVFAGGVGIIEITFTVPD | 98 |
| | ADTVIKALSVLKEKGASIGAGTVTSVEQCRKAVEAGAEYIVSPHLDEE | |
| | ISQFCKELGIPYMPGVMTPTELVKAMKLGHKVLKLFPGEVVGPQFVK | |
| | AMKKTYPDALFVPTGGVNLDNVCEWLKAGALAVGVGSALVKGTPD | |
| | EVREKAKAFVEKVKACGVNQMELVKKVFGT | |
| CompA.038 | MGNVEIIEKFAKEKIVAVLRANSVEEAIEKAVAVFAGGVGIIEITFTVPD | 99 |
| | ADTVIKALSVLKEKGASIGAGTVTSVEQCRKAVEAGAEYIVSPHLDEE | |
| | ISQFCKELGIPYMPGVMTPTELVKAMKLGHKVLKLFPGEVVGPQFVK | |
| | AMKKTYPDALFVPTGGVNLDNVCEWLKAGALAVGVGSALVKGTPD | |
| | EVREKAKAFVEKVRASGVNAMELVKKALGT | |
| CompA.039 | MGNPKEIIEKFAKEKIVAVLRANSVEEAIEKAVAVFAGGVGIIEITFTVPD | 100 |
| | ADTVIKALSVLKEKGASIGAGTVTSVEQCRKAVEAGAEYIVSPHLDEE | |
| | ISQFCKELGIPYMPGVMTPTELVKAMKLGHKVLKLFPGEVVGPQFVK | |
| | AMKKTYPEALFVPTGGVNLDNVCEWFKAGALAVGVGSALVKGTPDE | |
| | VREKAKAFVEKVKACGVNQMELVKKAEGT | |
| CompA.040 | MGNKEIGEKFAREKIVAVLRANSVEEAIEKAVAVFAGGVGIIEITFTVPD | 101 |
| | ADTVIKALSVLKEKGASIGAGTVTSVEQCRKAVEAGAEYIVSPHLDEE | |
| | ISQFCKELGIPYMPGVMTPTELVKAMKLGHKVLKLFPGEVVGPQFVK | |
| | AMKKTYPEALFVPTGGVNLDNVCEWLEAGALAVGVGSALVKGTPDE | |
| | VREKAKAFVEKVKACPFDSMELFKKAEGT | |
| CompA.041 | MGDLKMAKAFAREKIVAVLRANSVEEAIEKAVAVFAGGVGIIEITFTVP | 102 |
| | DADTVIKALSVLKEKGAKIGAGTVTSVEQCRKAVEAGADYIVSPHLD | |
| | EEISQFCKELGIPYMPGVMTPTELVKAMKLGHTILKLFPGEVVGPQFV | |
| | KAMKKTYPEALFVPTGGVNLDNVCEWLEAGALAVGVGSALVKGTPD | |
| | EVREKAKAFVAKVAACPVDAMELLKEAEAIMGT | |
| CompA.042 | MGDKKMAKAFAKEKIVAVLRANSVEEAIEKAVAVFAGGVGIIEITFTVP | 103 |
| | DADTVIKALSVLKEKGAKIGAGTVTSVEQCRKAVEAGADYIVSPHLD | |
| | EEISQFCKELGIPYMPGVMTPTELVKAMKLGHTILKLFPGEVVGPQFV | |
| | KAMKKTYPQALFVPTGGVNLDNVCEWLEAGALAVGVGSALVKGTP | |
| | DEVREKAKAFVAKVAACPYDPMKLVEEVEKLLGT | |
| CompA.043 | MGTDEKMAKAFAREKIVAVLRANSVEEAIEKAVAVFAGGVGIIEITFTV | 104 |
| | PDADTVIKALSVLKEKGAKIGAGTVTSVEQCRKAVEAGADYIVSPHL | |
| | DEEISQFCKELGIPYMPGVMTPTELVKAMKLGHTILKLFPGEVVGPQF | |
| | VKAMKKTYPQALFVPTGGVNLDNVCEWLKAGAIAVGVGSALVKGTP | |
| | DEVREKAKAFVAKVAACGVDPMAEVEKAKALEGT | |
| CompA.044 | MGDEKMAKAFAREKIVAVLRANSVEEAIEKAVAVFAGGVGIIEITFTVP | 105 |
| | DADTVIKALSVLKEKGAKIGAGTVTSVEQCRKAVEAGADYIVSPHLD | |
| | EEISQFCKELGIPYMPGVMTPTELVKAMKLGHTILKLFPGEVVGPQFV | |
| | KAMKKTYPEALFVPTGGVNLDNVCEWLDAGALAVGVGSALVKGTP | |
| | DEVREKAKAFVAKVAACPFDPMALVDKVLALFGT | |
| CompA.007 | KEVGKKLLLLVTDPADK | 106 |
| CompA.022 | DKESEKLIKLETDPAML | 107 |
| CompA.019 | EKEGKKALKLETDPAMK | 108 |
| CompA.026 | VQAVHKKALAVAPKLTEAQA | 109 |
| CompA.003 | ENKKVGEELIKLVTRPEDR | 110 |
| CompA.008 | VNNEVGKKLLLLVTDPADK | 111 |
| CompA.021 | ENEKEGKKALKLETDPAMK | 112 |
| CompA.023 | EAKKESEKLIKLETDPAML | 113 |
| CompA.027 | PEVEAVHEKALAVAPKLTEAQA | 114 |
| CompA.045 | MGSSHHHHHHGSGSEADLKMLKKLYEERIVAVLRANSVEEAIEKAVA | 115 |
| | VFAGGVKIIEITFTVPDADTVIKALSVLKEKGAIIGAGTVTSVEQCRKA | |
| | VEAGAEYIVSPHLDEEISQFCKEKGVPYMPGVMTPTELVKAMKLGHK | |
| | LLKLFPGEVVGPQFVKAMKKTYPEAAFVPTGGVNLDNVCEWIEAGA | |
| | VAVGVGSALVKGTPDEVREKAKAFVEKARLCAGKYKEQLDKQLALQ | |
| | LGT | |
| CompA.046 | MGSSHHHHHHGSGSEADLKMLKKLYQERIVAVLRANSVEEAIEKAVA | 116 |
| | VFAGGVKIIEITFTVPDADTVIKALSVLKEKGAIIGAGTVTSVEQCRKA | |
| | VEAGAEYIVSPHLDEEISQFCKEKGVPYMPGVMTPTELVKAMKLGHK | |
| | LLKLFPGEVVGPQFVKAMKKTYPEAAFVPTGGVNLDNVCEWIEAGA | |
| | VAVGVGSALVKGTPDEVREKAKAFVEKANLCAGKYKKQLDKQLALQ | |
| | LGT | |
| CompA.047 | MGSSHHHHHHGSGSEADLKMLKKLYTEKIVAVLRANSVEEAIEKAVA | 117 |
| | VFAGGVKIIEITFTVPDADTVIKALSVLKEKGAIIGAGTVTSVEQCRKA | |
| | VEAGAEYIVSPHLDEEISQFCKEKGVPYMPGVMTPTELVKAMKLGHK | |
| | LLKLFPGEVVGPQFVKAMKKTYPEAAFVPTGGVNLDNVCEWIEAGA | |
| | VAVGVGSALVKGTPDEVREKAKAFVQKANECAGKYSDQLDKQLKLQ | |
| | LGT | |
| CompA.048 | MGSSHHHHHHGSGSEADLKMLKKLYTEKIVAVLRANSVEEAIEKAVA | 118 |
| | VFAGGVKIIEITFTVPDADTVIKALSVLKEKGAIIGAGTVTSVEQCRKA | |
| | VEAGAEYIVSPHLDEEISQFCKEKGVPYMPGVMTPTELVKAMKLGHK | |
| | LLKLFPGEVVGPQFVKAMKKTYPEAAFVPTGGVNLDNVCEWIEAGA | |
| | VAVGVGSALVKGTPDEVREKAKAFVQKANECAGKYSSQLDKQLSLQ | |
| | LGT | |
| CompA.052 | MGSSHHHHHHGSGGHKLLKLFPGEVVGPQFVKAMKKTFPDAAFVPT | 119 |
| | GGVNLDNVCEWFKAGVLAVGVGSALVKGTPDEVREKAKAFVEKAN | |
| | RCSEEAEKESEKLIKLETDPAMLRMAKIFGKEKIVAVLRANSVEEAIEK | |
| | AVAVFAGGVTVIEITFTVPDADTVIKALSVLKEKGAVIGAGTVTSVEQC | |
| | RKAVEAGAEYIVSPHLDEEISQFCKEKGIPYMPGVMTPTELVKAMKLG | |
| | SGSGT | |
| CompA.053 | MGSSHHHHHHGSTEAQALMLKAFVEEKIVAVLRANSVEEAIEKAVAV | 120 |
| | FAGGVNIIEITFTVPDADTVIKALSVLKEKGAIIGAGTVTSVEQCRKAV | |
| | EAGADYIVSPHLDEEISQFCKEVGVAYMPGVMTPTELVKAMKLGHLV | |
| | LKLFPGEVVGPQFVKAMKKTFPDVFFVPTGGVNLDNVCEWFKAGVL | |
| | AVGVGSALVKGTPDEVREKAKAFVDKVTACIDAEVNAVHKKALAVAP | |
| | KGT | |
| CompA.058 | MGSSHHHHHHGSGSGNEEIEEKFASEKIVAVLRANSVEEAIEKAVAVFA | 121 |
| | GGVGIIEITFTVPDADTVIKALSVLKEKGASIGAGTVTSVEQCRKAVEA | |
| | GAEYIVSPHLDEEISQFCKELGIPYMPGVMTPTELVKAMKLGHKVLKL | |
| | FPGEVVGPQFVKAMKKTYPEALFVPTGGVNLDNVCEWLKAGAIAVG | |
| | VGSALVKGTPDEVREKAKAFVEKVSACGVDSMAILSKVKGT | |
| CompA.059 | MGSSHHHHHHGSGSGNEEIEEKFAKEKIVAVLRANSVEEAIEKAVAVF | 122 |
| | AGGVGIIEITFTVPDADTVIKALSVLKEKGASIGAGTVTSVEQCRKAVE | |
| | AGAEYIVSPHLDEEISQFCKELGIPYMPGVMTPTELVKAMKLGHKVL | |
| | KLFPGEVVGPQFVKAMKKTYPEALFVPTGGVNLDNVCEWLKAGAIA | |
| | VGVGSALVKGTPDEVREKAKAFVEKVSACGVDSMAILEKVKGT | |
| CompA.060 | MGSSHHHHHHGSGNVEIIEKFAKEKIVAVLRANSVEEAIEKAVAVFAG | 123 |
| | GVGIIEITFTVPDADTVIKALSVLKEKGASIGAGTVTSVEQCRKAVEAG | |
| | AEYIVSPHLDEEISQFCKELGIPYMPGVMTPTELVKAMKLGHKVLKLF | |
| | PGEVVGPQFVKAMKKTYPDALFVPTGGVNLDNVAEWLKAGALAVG | |
| | VGSALVKGTPDEVREKAKAFVEKVNASGVNAMKLVEKALGT | |
| CompA.061 | MGSSHHHHHHGSGNVEIIEKFASEKIVAVLRANSVEEAIEKAVAVFAGG | 124 |
| | VGIIEITFTVPDADTVIKALSVLKEKGASIGAGTVTSVEQCRKAVEAGA | |
| | EYIVSPHLDEEISQFCKELGIPYMPGVMTPTELVKAMKLGHKVLKLFP | |
| | GEVVGPQFVKAMKKTYPDALFVPTGGVNLDNVAEWLKAGALAVGV | |
| | GSALVKGTPDEVREKAKAFVEKVSASGVNSMELVKKALGT | |
| CompA.062 | MGSSHHHHHHGSGNVEIIEKFASEKIVAVLRANSVEEAIEKAVAVFAGG | 125 |
| | VGIIEITFTVPDADTVIKALSVLKEKGASIGAGTVTSVEQCRKAVEAGA | |
| | EYIVSPHLDEEISQFCKELGIPYMPGVMTPTELVKAMKLGHKVLKLFP | |
| | GEVVGPQFVKAMKKTYPDALFVPTGGVNLDNVAEWLKAGALAVGV | |
| | GSALVKGTPDEVREKAKAFVEKVSASGVNAMELVKKALGT | |
| CompA.065 | MGSSHHHHHHGSGNPKEIIEKFASEKIVAVLRANSVEEAIEKAVAVFAG | 126 |
| | GVGIIEITFTVPDADTVIKALSVLKEKGASIGAGTVTSVEQCRKAVEAG | |
| | AEYIVSPHLDEEISQFCKELGIPYMPGVMTPTELVKAMKLGHKVLKLF | |
| | PGEVVGPQFVKAMKKTYPEALFVPTGGVNLDNVCEWFKAGALAVG | |
| | VGSALVKGTPDEVREKAKAFVEKVSACGVNQMELVKKAEGT | |
| CompA.066 | MGSSHHHHHHGSGNPKEIIEKFASEKIVAVLRANSVEEAIEKAVAVFAG | 127 |
| | GVGIIEITFTVPDADTVIKALSVLKEKGASIGAGTVTSVEQCRKAVEAG | |
| | AEYIVSPHLDEEISQFCKELGIPYMPGVMTPTELVKAMKLGHKVLKLF | |
| | PGEVVGPQFVKAMKKTYPEALFVPTGGVNLDNVCEWFKAGALAVG | |
| | VGSALVKGTPDEVREKAKAFVEKVSACGVNSMELVKKAEGT | |
| CompA.067 | MGSSHHHHHHGSGSGNKEIGEKFAEEKIVAVLRANSVEEAIEKAVAVF | 128 |
| | AGGVGIIEITFTVPDADTVIKALSVLKEKGASIGAGTVTSVEQCRKAVE | |
| | AGAEYIVSPHLDEEISQFCKELGIPYMPGVMTPTELVKAMKLGHKVL | |
| | KLFPGEVVGPQFVKAMKKTYPEALFVPTGGVNLDNVCEWLEAGALA | |
| | VGVGSALVKGTPDEVREKAKAFVEKVNACPFNSMELFRKAEGT | |
| CompA.068 | MGSSHHHHHHGSGSGNKEIGEKFATEKIVAVLRANSVEEAIEKAVAVF | 129 |
| | AGGVGIIEITFTVPDADTVIKALSVLKEKGASIGAGTVTSVEQCRKAVE | |
| | AGAEYIVSPHLDEEISQFCKELGIPYMPGVMTPTELVKAMKLGHKVL | |
| | KLFPGEVVGPQFVKAMKKTYPEALFVPTGGVNLDNVCEWLEAGALA | |
| | VGVGSALVKGTPDEVREKAKAFVEKVRRCPFDSQELFKKAEGT | |
| CompA.069 | MGSSHHHHHHGSGSGNKEIGEKFATEKIVAVLRANSVEEAIEKAVAVF | 130 |
| | AGGVGIIEITFTVPDADTVIKALSVLKEKGASIGAGTVTSVEQCRKAVE | |
| | AGAEYIVSPHLDEEISQFCKELGIPYMPGVMTPTELVKAMKLGHKVL | |
| | KLFPGEVVGPQFVKAMKKTYPEALFVPTGGVNLDNVCEWLEAGALA | |
| | VGVGSALVKGTPDEVREKAKAFVEKVKACPFDSMELFKKAEGT | |
| CompA.070 | MGSSHHHHHHGSGSGNKEIGEKFASEKIVAVLRANSVEEAIEKAVAVF | 131 |
| | AGGVGIIEITFTVPDADTVIKALSVLKEKGASIGAGTVTSVEQCRKAVE | |
| | AGAEYIVSPHLDEEISQFCKELGIPYMPGVMTPTELVKAMKLGHKVL | |
| | KLFPGEVVGPQFVKAMKKTYPEALFVPTGGVNLDNVCEWLEAGALA | |
| | VGVGSALVKGTPDEVREKAKAFVEKVSACPFDSMELFKKAEGT | |
| CompA.074 | MGSSHHHHHHGSGSGNEEMEELFASHKIVAVLRANSVEEAIEKAVAVF | 132 |
| | AGGVHLIEITFTVPDADTVIKALSVLKEKGAIIGAGTVTSVEQCRKAVE | |
| | SGAEFIVSPHLDEEISQFCKEKGVFYMPGVMTPTELVKAMKLGHKVL | |
| | KLFPGEVVGPQFVKAMKGPFPEVLFVPTGGVNLDNVCEWLKAGVIA | |
| | VGVGSALVKGTPDEVREKAKAFVEKVSACGVDSMAILSKVKGT | |
| CompA.075 | MGSSHHHHHHGSGSGNEEMEELFAKHKIVAVLRANSVEEAIEKAVAVF | 133 |
| | AGGVHLIEITFTVPDADTVIKALSVLKEKGAIIGAGTVTSVEQCRKAVE | |
| | SGAEFIVSPHLDEEISQFCKEKGVFYMPGVMTPTELVKAMKLGHKVL | |
| | KLFPGEVVGPQFVKAMKGPFPEVLFVPTGGVNLDNVCEWLKAGVIA | |
| | VGVGSALVKGTPDEVREKAKAFVEKVSACGVDSMAILEKVKGT | |
| CompA.076 | MGSSHHHHHHGSGSGNEEMEELFASHKIVAVLRANSVEEAIEKAVAVF | 134 |
| | AGGVHLIEITFTVPDADTVIKALSVLKEKGAIIGAGTVTSVEQCRKAVE | |
| | SGAEFIVSPHLDEEISQFCKEKGVFYMPGVMTPTELVKAMKLGHKVL | |
| | KLFPGEVVGPQFVKAMKGPFPEVLFVPTGGVNLDNVCEWFKAGVIAV | |
| | GVGSALVKGTPDEVREKAKAFVEKISACGVDSMAILSKVKGT | |
| CompA.077 | MGSSHHHHHHGSGSGNEEMEELFAKHKIVAVLRANSVEEAIEKAVAVF | 135 |
| | AGGVHLIEITFTVPDADTVIKALSVLKEKGAIIGAGTVTSVEQCRKAVE | |
| | SGAEFIVSPHLDEEISQFCKEKGVFYMPGVMTPTELVKAMKLGHKVL | |
| | KLFPGEVVGPQFVKAMKGPFPEVLFVPTGGVNLDNVCEWFKAGVIAV | |
| | GVGSALVKGTPDEVREKAKAFVEKISACGVDSMAILEKVKGT | |
| CompA.078 | MGSSHHHHHHGSGSEADLKMLKLFYQHRIVAVLRANSVEEAIEKAVA | 136 |
| | VFAGGVHLIEITFTVPDADTVIKALSVLKEKGAIIGAGTVTSVEQCRKA | |
| | VESGAEFIVSPHLDEEISQFCKEKGVFYMPGVMTPTELVKAMKLGHKI | |
| | LKLFPGEVVGPQFVKAMKGPFPEVAFVPTGGVNLDNVCEWIEAGAVA | |
| | VGVGSALVKGTPDEVREKAKAFVEKANLCAGKYKKQLDKQLALQL | |
| | GT | |
| CompA.079 | MGSSHHHHHHGSGSEADLKMLKLFYQHRIVAVLRANSVEEAIEKAVA | 137 |
| | VFAGGVHLIEITFTVPDADTVIKALSVLKEKGAIIGAGTVTSVEQCRKA | |
| | VESGAEFIVSPHLDEEISQFCKEKGVFYMPGVMTPTELVKAMKLGHKI | |
| | LKLFPGEVVGPQFVKAMKGPFPEVAFVPTGGVNLDNVCEWFEAGVV | |
| | AVGVGSALVKGTPDEVREKAKAFVEKINLCAGKYKKQLDKQLALQL | |
| | GT | |
| CompA.082 | MGSSHHHHHHGSGGHKILKLFPGEVVGPQFVKAMKGPFPDVAFVPTG | 138 |
| | GVNLDNVCEWFKAGVLAVGVGSALVKGTPDEVREKAKAFVEKINRC | |
| | SEEAEKESEKIIKLETDPAALRMAKLFGKHKIVAVLRANSVEEAIEKAV | |
| | AVFAGGVHLIEITFTVPDADTVIKALSVLKEKGAIIGAGTVTSVEQCRK | |
| | AVESGAEFIVSPHLDEEISQFCKEKGVFYMPGVMTPTELVKAMKLGSG | |
| | SGT | |
| CompA.083 | MGSSHHHHHHGSGSEADLKMLKKFYTHKIVAVLRANSVEEAIEKAVA | 139 |
| | VFAGGVHLIEITFTVPDADTVIKALSVLKEKGAIIGAGTVTSVEQCRKA | |
| | VESGAEFIVSPHLDEEISQFCKEKGVPYMPGVMTPTELVKAMKLGHK | |
| | LLKLFPGEVVGPQFVKAMKGPFPEVAFVPTGGVNLDNVCEWIEAGVV | |
| | AVGVGSALVKGTPDEVREKAKAFVQKANECAGKYSDQLDKQLKLQL | |
| | GT | |
| CompA.085 | MGSSHHHHHHGSGSEADLKMLKKFYTHKIVAVLRANSVEEAIEKAVA | 140 |
| | VFAGGVHLIEITFTVPDADTVIKALSVLKEKGAIIGAGTVTSVEQCRKA | |
| | VESGAEFIVSPHLDEEISQFCKEKGVPYMPGVMTPTELVKAMKLGHK | |
| | LLKLFPGEVVGPQFVKAMKGPFPEVAFVPTGGVNLDNVCEWIEAGVV | |
| | AVGVGSALVKGTPDEVREKAKAFVQKANECAGKYSSQLDKQLSLQL | |
| | GT | |
| CompA.101 | MGSSHHHHHHGSTEAQALMLKLFVEHKIVAVLRANSVEEAIEKAVAV | 141 |
| | FAGGVHLIEITFTVPDADTVIKALSVLKEKGAIIGAGTVTSVEQCRKAV | |
| | ESGAEFIVSPHLDEEISQFCKEKGVFYMPGVMTPTELVKAMKLGHLVL | |
| | KLFPGEVVGPQFVKAMKGPFPDVFFVPTGGVNLDNVCEWFKAGVLA | |
| | VGVGSALVKGTPDEVREKAKAFVDKVTACIDAEVNAVHKKALAVAP | |
| | KGT | |
| CompA.117 | MGSSHHHHHHGSGNPKEMIELFASHKIVAVLRANSVEEAIEKAVAVFA | 142 |
| | GGVHLIEITFTVPDADTVIKALSVLKEKGAIIGAGTVTSVEQCRKAVES | |
| | GAEFIVSPHLDEEISQFCKEKGVFYMPGVMTPTELVKAMKLGHKVLK | |
| | LFPGEVVGPQFVKAMKGPFPEVLFVPTGGVNLDNVCEWFKAGVLAV | |
| | GVGSALVKGTPDEVREKAKAFVEKISACGVNSMELVKKAEGT | |
| CompA.118 | MGSSHHHHHHGSGSGNKEMGELFATHKIVAVLRANSVEEAIEKAVAVF | 143 |
| | AGGVHLIEITFTVPDADTVIKALSVLKEKGAIIGAGTVTSVEQCRKAVE | |
| | SGAEFIVSPHLDEEISQFCKEKGVFYMPGVMTPTELVKAMKLGHKILK | |
| | LFPGEVVGPQFVKAMKGPFPEVLFVPTGGVNLDNVCEWLEAGVLAV | |
| | GVGSALVKGTPDEVREKAKAFVEKVKACPFDSMELFKKAEGT | |
| CompA.119 | MGSSHHHHHHGSGSGNKEMGELFASHKIVAVLRANSVEEAIEKAVAV | 144 |
| | FAGGVHLIEITFTVPDADTVIKALSVLKEKGAIIGAGTVTSVEQCRKAV | |
| | ESGAEFIVSPHLDEEISQFCKEKGVFYMPGVMTPTELVKAMKLGHKIL | |
| | KLFPGEVVGPQFVKAMKGPFPEVLFVPTGGVNLDNVCEWLEAGVLA | |
| | VGVGSALVKGTPDEVREKAKAFVEKVSACPFDSMELFKKAEGT | |
| CompA.120 | MGSSHHHHHHGSGSGNKEMGELFATHKIVAVLRANSVEEAIEKAVAVF | 145 |
| | AGGVHLIEITFTVPDADTVIKALSVLKEKGAIIGAGTVTSVEQCRKAVE | |
| | SGAEFIVSPHLDEEISQFCKEKGVFYMPGVMTPTELVKAMKLGHKILK | |
| | LFPGEVVGPQFVKAMKGPFPEVLFVPTGGVNLDNVCEWFEAGVLAV | |
| | GVGSALVKGTPDEVREKAKAFVEKIKACPFDSMELFKKAEGT | |
| CompA.121 | MGSSHHHHHHGSGSGNKEMGELFASHKIVAVLRANSVEEAIEKAVAV | 146 |
| | FAGGVHLIEITFTVPDADTVIKALSVLKEKGAIIGAGTVTSVEQCRKAV | |
| | ESGAEFIVSPHLDEEISQFCKEKGVFYMPGVMTPTELVKAMKLGHKIL | |
| | KLFPGEVVGPQFVKAMKGPFPEVLFVPTGGVNLDNVCEWFEAGVLA | |
| | VGVGSALVKGTPDEVREKAKAFVEKISACPFDSMELFKKAEGT | |
| CompA.123 | MGSSHHHHHHGSGSEADLKMLKKLYQERIVAVLRANSVEEAIEKAVA | 147 |
| | VFAGGVKIIEITFTVPDADTVIKALSVLKEKGAIIGAGTVTSVEQCRKA | |
| | VESGAEFIVSPHLDEEISQFCKEKGVPYMPGVMTPTELVKAMKLGHK | |
| | LLKLFPGEVVGPQFVKAMKGPFPEVAFVPTGGVNLDNVCEWIEAGVV | |
| | AVGVGSALVKGTPDEVREKAKAFVEKANLCAGKYKKQLDKQLALQL | |
| | GT | |
| CompA.124 | MGSSHHHHHHGSGSEADLKMLKKLYTEKIVAVLRANSVEEAIEKAVA | 148 |
| | VFAGGVKIIEITFTVPDADTVIKALSVLKEKGAIIGAGTVTSVEQCRKA | |
| | VESGAEFIVSPHLDEEISQFCKEKGVPYMPGVMTPTELVKAMKLGHK | |
| | LLKLFPGEVVGPQFVKAMKGPFPEVAFVPTGGVNLDNVCEWIEAGVV | |
| | AVGVGSALVKGTPDEVREKAKAFVQKANECAGKYSDQLDKQLKLQL | |
| | GT | |
| CompA.135 | MGSSHHHHHHGSGSGNEEIEEKFASEKIVAVLRANSVEEAIEKAVAVFA | 149 |
| | GGVGIIEITFTVPDADTVIKALSVLKEKGASIGAGTVTSVEQCRKAVES | |
| | GAEFIVSPHLDEEISQFCKEKGVPYMPGVMTPTELVKAMKLGHKVLK | |
| | LFPGEVVGPQFVKAMKGPFPEVLFVPTGGVNLDNVCEWLKAGVIAV | |
| | GVGSALVKGTPDEVREKAKAFVEKVSACGVDSMAILSKVKGT | |
| CompA.137 | MGSSHHHHHHGSGNVEIIEKFAKEKIVAVLRANSVEEAIEKAVAVFAG | 150 |
| | GVGIIEITFTVPDADTVIKALSVLKEKGASIGAGTVTSVEQCRKAVESG | |
| | AEFIVSPHLDEEISQFCKEKGVPYMPGVMTPTELVKAMKLGHKVLKL | |
| | FPGEVVGPQFVKAMKGPFPDVLFVPTGGVNLDNVAEWLKAGVLAVG | |
| | VGSALVKGTPDEVREKAKAFVEKVNASGVNAMKLVEKALGT | |
| CompA.138 | MGSSHHHHHHGSGNVEIIEKFASEKIVAVLRANSVEEAIEKAVAVFAGG | 151 |
| | VGIIEITFTVPDADTVIKALSVLKEKGASIGAGTVTSVEQCRKAVESGA | |
| | EFIVSPHLDEEISQFCKEKGVPYMPGVMTPTELVKAMKLGHKVLKLFP | |
| | GEVVGPQFVKAMKGPFPDVLFVPTGGVNLDNVAEWLKAGVLAVGV | |
| | GSALVKGTPDEVREKAKAFVEKVSASGVNSMELVKKALGT | |
| CompA.139 | MGSSHHHHHHGSGNVEIIEKFASEKIVAVLRANSVEEAIEKAVAVFAGG | 152 |
| | VGIIEITFTVPDADTVIKALSVLKEKGASIGAGTVTSVEQCRKAVESGA | |
| | EFIVSPHLDEEISQFCKEKGVPYMPGVMTPTELVKAMKLGHKVLKLFP | |
| | GEVVGPQFVKAMKGPFPDVLFVPTGGVNLDNVAEWLKAGVLAVGV | |
| | GSALVKGTPDEVREKAKAFVEKVSASGVNAMELVKKALGT | |
| CompA.142 | MGSSHHHHHHGSGNPKEIIEKFASEKIVAVLRANSVEEAIEKAVAVFAG | 153 |
| | GVGIIEITFTVPDADTVIKALSVLKEKGASIGAGTVTSVEQCRKAVESG | |
| | AEFIVSPHLDEEISQFCKEKGVPYMPGVMTPTELVKAMKLGHKVLKL | |
| | FPGEVVGPQFVKAMKGPFPEVLFVPTGGVNLDNVCEWFKAGVLAVG | |
| | VGSALVKGTPDEVREKAKAFVEKVSACGVNQMELVKKAEGT | |
| CompA.143 | MGSSHHHHHHGSGNPKEIIEKFASEKIVAVLRANSVEEAIEKAVAVFAG | 154 |
| | GVGIIEITFTVPDADTVIKALSVLKEKGASIGAGTVTSVEQCRKAVESG | |
| | AEFIVSPHLDEEISQFCKEKGVPYMPGVMTPTELVKAMKLGHKVLKL | |
| | FPGEVVGPQFVKAMKGPFPEVLFVPTGGVNLDNVCEWFKAGVLAVG | |
| | VGSALVKGTPDEVREKAKAFVEKVSACGVNSMELVKKAEGT | |
| CompA.144 | MGSSHHHHHHGSGSGNKEIGEKFAKEKIVAVLRANSVEEAIEKAVAVF | 155 |
| | AGGVGIIEITFTVPDADTVIKALSVLKEKGASIGAGTVTSVEQCRKAVE | |
| | SGAEFIVSPHLDEEISQFCKEKGVPYMPGVMTPTELVKAMKLGHKVL | |
| | KLFPGEVVGPQFVKAMKGPFPEVLFVPTGGVNLDNVCEWLEAGVLA | |
| | VGVGSALVKGTPDEVREKAKAFVEKVNACPFNSMELFRKAEGT | |
| CompA.145 | MGSSHHHHHHGSGSGNKEIGEKFATEKIVAVLRANSVEEAIEKAVAVF | 156 |
| | AGGVGIIEITFTVPDADTVIKALSVLKEKGASIGAGTVTSVEQCRKAVE | |
| | SGAEFIVSPHLDEEISQFCKEKGVPYMPGVMTPTELVKAMKLGHKVL | |
| | KLFPGEVVGPQFVKAMKGPFPEVLFVPTGGVNLDNVCEWLEAGVLA | |
| | VGVGSALVKGTPDEVREKAKAFVEKVRRCPFDSQELFKKAEGT | |
| CompA.146 | MGSSHHHHHHGSGSGNKEIGEKFATEKIVAVLRANSVEEAIEKAVAVF | 157 |
| | AGGVGIIEITFTVPDADTVIKALSVLKEKGASIGAGTVTSVEQCRKAVE | |
| | SGAEFIVSPHLDEEISQFCKEKGVPYMPGVMTPTELVKAMKLGHKVL | |
| | KLFPGEVVGPQFVKAMKGPFPEVLFVPTGGVNLDNVCEWLEAGVLA | |
| | VGVGSALVKGTPDEVREKAKAFVEKVKACPFDSMELFKKAEGT | |
| CompA.147 | MGSSHHHHHHGSGSGNKEIGEKFASEKIVAVLRANSVEEAIEKAVAVF | 158 |
| | AGGVGIIEITFTVPDADTVIKALSVLKEKGASIGAGTVTSVEQCRKAVE | |
| | SGAEFIVSPHLDEEISQFCKEKGVPYMPGVMTPTELVKAMKLGHKVL | |
| | KLFPGEVVGPQFVKAMKGPFPEVLFVPTGGVNLDNVCEWLEAGVLA | |
| | VGVGSALVKGTPDEVREKAKAFVEKVSACPFDSMELFKKAEGT | |

### INCORPORATION BY REFERENCE

The entire disclosure of each of the patent and scientific documents referred to herein is incorporated by reference for all purposes.

The reference in this specification to any prior publication (or information derived from it), or to any matter which is known, is not, and should not be taken as an acknowledgment or admission or any form of suggestion that prior publication (or information derived from it) or known matter forms part of the common general knowledge in the field of endeavor to which this specification relates.

### EQUIVALENTS

The invention may be embodied in other specific forms without departing from the spirit or essential characteristics thereof. The foregoing embodiments are therefore to be considered in all respects illustrative rather than limiting on the invention described herein. The scope of the invention is thus indicated by the appended claims rather than by the foregoing description, and all changes that come within the meaning and range of equivalency of the claims are intended to be embraced therein.

1. A polypeptide that is a circular permutation of 153-50A, comprising an assembly domain,
the assembly domain comprising, in N- to C-terminal order, a N-terminal polypeptide segment, a linking polypeptide segment, and a C-terminal polypeptide segment, wherein:
I. a) the N-terminal polypeptide segment comprises residues 74-201 of SEQ ID NO: 1 or a variant thereof, and the C-terminal polypeptide segment comprises residues 1-73 of SEQ ID NO: 1 or a variant thereof;
II. b) the N-terminal polypeptide segment comprises residues 107-201 of SEQ ID NO: 1 or a variant thereof, and the C-terminal polypeptide segment comprises residues 1-106 of SEQ ID NO: 1 or a variant thereof; or
III. c) the N-terminal polypeptide segment comprises residues 128-201 of SEQ ID NO: 1 or a variant thereof, and the C-terminal polypeptide segment comprises residues 1-127 of SEQ ID NO: 1 or a variant thereof;
wherein variants thereof are at least 50%, at least 60%, at least 70%, at least 75%, at least 80%, at least 85%, at least 90%, at least 95%, at least 96%, at least 97%, at least 98%, at least 99% identical to the reference sequence.

2. The polypeptide of claim 1,
wherein the N-terminal polypeptide segment and the C-terminal polypeptide segment comprises polypeptide sequences each selected from pairs A, B, or C, or from variants thereof at least 50%, at least 60%, at least 70%, at least 75%, at least 80%, at least 85%, at least 90%, at least 95%, at least 96%, at least 97%, at least 98%, at least 99% identical thereto:

| | N-terminal polypeptide segment | C-terminal polypeptide segment |
|---|---|---|
| A | MKMEELFKKHKIVAVLRANSVEEAIEKAVAVFAG | EQCRKAVESGAEFIVSPHLDEEISQFCKEKGVFYM |
| | GVHLIEITFTVPDADTVIKALSVLKEKGAIIGAGTV | PGVMTPTELVKAMKLGHDILKLFPGEVVGPQFV |
| | TSV (SEQ ID NO: 2) | KAMKGPFPNVKFVPTGGVNLDNVCKWFKAGVL |
| | | AVGVGKALVKGKPDEVREKAKKFVKKIR (SEQ ID |
| | | NO: 5) |
| B | MKMEELFKKHKIVAVLRANSVEEAIEKAVAVFAG | YMPGVMTPTELVKAMKLGHDILKLFPGEVVGPQ |
| | GVHLIEITFTVPDADTVIKALSVLKEKGAIIGAGTV | FVKAMKGPFPNVKFVPTGGVNLDNVCKWFKAG |
| | TSVEQCRKAVESGAEFIVSPHLDEEISQFCKEKGV | VLAVGVGKALVKGKPDEVREKAKKFVKKIR (SEQ |
| | F (SEQ ID NO: 3) | ID NO: 6) |
| C | MKMEELFKKHKIVAVLRANSVEEAIEKAVAVFAG | LKLFPGEVVGPQFVKAMKGPFPNVKFVPTGGVN |
| | GVHLIEITFTVPDADTVIKALSVLKEKGAIIGAGTV | LDNVCKWFKAGVLAVGVGKALVKGKPDEVREKA |
| | TSVEQCRKAVESGAEFIVSPHLDEEISQFCKEKGV | KKFVKKIR (SEQ ID NO: 7) |
| | FYMPGVMTPTELVKAMKLGHDI (SEQ ID NO: 4) | |

3. The polypeptide of claim 1, wherein the linking polypeptide segment comprises a polypeptide sequence at least 50%, at least 60%, at least 70%, at least 75%, at least 80%, at least 85%, at least 90%, at least 95%, at least 96%, at least 97%, at least 98%, at least 99%, or 100% identical to any of one:
(SEQ ID NO: 8)
KKVGEELIKLVTRPEDR;
(SEQ ID NO: 9)
KEVGKKLLLLVTDPADKK;
(SEQ ID NO: 10)
DKESEKLIKLETDPAMLRM;
(SEQ ID NO: 11)
EKEGKKALKLETDPAMKKMV;
(SEQ ID NO: 12)
VQAVHKKALAVAPKLTEAQALM;
(SEQ ID NO: 13)
ENKKVGEELIKLVTRPED;
(SEQ ID NO: 14)
VNNEVGKKLLLLVTDPAD;
(SEQ ID NO: 15)
ENEKEGKKALKLETDPAM;
(SEQ ID NO: 16)
EAKKESEKLIKLETDPAM;
(SEQ ID NO: 17)
PEVEAVHEKALAVAPKLTEAQ;
(SEQ ID NO: 18)
ENKKVGEELIKLVTRPED;
(SEQ ID NO: 19)
ENEKEGKKALKLETDPAM;
(SEQ ID NO: 20)
EAKKESEKLIKLETDPAM;
or
(SEQ ID NO: 21)
PEVEAVHEKALAVAPKLTEAQ.

4. The polypeptide of claim 1, wherein the assembly domain comprises a polypeptide sequence at least 50%, at least 60%, at least 70%, at least 75%, at least 80%, at least 85%, at least 90%, at least 95%, at least 96%, at least 97%, at least 98%, at least 99%, or 100% identical to an one of:
a)
(SEQ ID NO: 22)
XXXXXVEQCRKAVESGAEFIVSPHLDEEISQFCKEKGVFYMPGVMTPTE
LVKAMKLGHDILKLFPGEVVGPQFVKAMKGPFPNVKFVPTGGVNLDNVC
KWFKAGVLAVGVGKALVKGKPDEVREKAKKFVKKIRGCTEGTXXXXXXX
XXXXXXXXXMKMEELFKKHKIVAVLRANSVEEAIEKAVAVFAGGVHLIE
ITFTVPDADTVIKALSVLKEKGAIIGAGTVTS;
b)
(SEQ ID NO: 23)
XXXXXXYMPGVMTPTELVKAMKLGHDILKLFPGEVVGPQFVKAMKGPFP
NVKFVPTGGVNLDNVCKWFKAGVLAVGVGKALVKGKPDEVREKAKKFVK
KIRGCTEXXXXXXXXXXXXXXXXXXMKMEELFKKHKIVAVLRANSVEEA
IEKAVAVFAGGVHLIEITFTVPDADTVIKALSVLKEKGAIIGAGTVTSV
EQCRKAVESGAEFIVSPHLDEEISQFCKEKGVF;
or
c)
(SEQ ID NO: 24)
XXXXXXXXLKLFPGEVVGPQFVKAMKGPFPNVKFVPTGGVNLDNVCKWF
KAGVLAVGVGKALVKGKPDEVREKAKKFVKKIRGCTEXXXXXXXXXXXX
XXXXXXMKMEELFKKHKIVAVLRANSVEEAIEKAVAVFAGGVHLIEITF
TVPDADTVIKALSVLKEKGAIIGAGTVTSVEQCRKAVESGAEFIVSPHL
DEEISQFCKEKGVFYMPGVMTPTELVKAMKLGHDI
and/or
IV. wherein the assembly domain comprises a polypeptide sequence at least 50%, at least 60%, at least 70%, at least 75%, at least 80%, at least 85%, at least 90%, at least 95%, at least 96%, at least 97%, at least 98%, at least 99%, or 100% identical to a first polypeptide sequence in Table 2 or Table 4.

5. (canceled)

6. A polypeptide that is a variant of I53-50A having a C-terminal extension, comprising an assembly domain, the assembly domain comprising, in N- to C-terminal order, a base polypeptide segment and an extending polypeptide segment,
Wherein the base polypeptide segment comprises a polypeptide sequence at least 50%, at least 60%, at least 70%, at least 75%, at least 80%, at least 85%, at least 90%, at least 95%, at least 96%, at least 97%, at least 98%, at least 99% identical to residues 1-201 of SEQ ID NO: 1.

7. The polypeptide of claim 6, wherein the extending polypeptide segment comprises a polypeptide sequence at least 50%, at least 60%, at least 70%, at least 75%, at least 80%, at least 85%, at least 90%, at least 95%, at least 96%, at least 97%, at least 98%, at least 99%, or 100% identical to any of one:
(SEQ ID NO: 33)
ENHARFAALRAELAGT;
(SEQ ID NO: 34)
ENPELTKEVAAFLAGT;
(SEQ ID NO: 35)
EYSEQFEARKKKLEGT;
(SEQ ID NO: 36)
RIDEEYQKRLEKLRGT;
(SEQ ID NO: 37)
KYKEQLDKQLKLQLGT;
(SEQ ID NO: 38)
KEKEYFEEQLEKLKGT;
(SEQ ID NO: 39)
YSKARLAEIKKALAGT;
(SEQ ID NO: 40)
AADEHMAAIMAALKGT;
(SEQ ID NO: 41)
VGDKLLAELKAQLAGT;
(SEQ ID NO: 42)
YLKANAEKLHKLLAGT;
(SEQ ID NO: 43)
ILAKLKAKILAKLKGT;
(SEQ ID NO: 44)
YSQATLKEILKALAGT;
(SEQ ID NO: 45)
FSKEACKKAILETKDGSGSGT;
(SEQ ID NO: 46)
PEVQAVHKKALAVAPKGT;
(SEQ ID NO: 47)
EEVKAVQQKALALAPKLTGSGSGT;
(SEQ ID NO: 48)
AEVAANQAKALSLAPPEAGGT;
(SEQ ID NO: 49)
AEVEANQAKALSLAPAPAGSGSGT;
(SEQ ID NO: 50)
EEVEAVQKKALSIAEELEGSGSGT;
(SEQ ID NO: 51)
EEVAAVQKKALSLAPKEPSGT;
(SEQ ID NO: 52)
DTLALLKERKGT;
(SEQ ID NO: 53)
DSMAILEKVKGT;
(SEQ ID NO: 54)
NQMELVKKVFGT;
(SEQ ID NO: 55)
NAMELVKKALGT;
(SEQ ID NO: 56)
NQMELVKKAEGT;
(SEQ ID NO: 57)
DSMELFKKAEGT;
(SEQ ID NO: 58)
DAMELLKEAEAIMGT;
(SEQ ID NO: 59)
DPMKLVEEVEKLLGT;
(SEQ ID NO: 60)
DPMAEVEKAKALEGT;
or
(SEQ ID NO: 61)
DPMALVDKVLALFGT.

8. The polypeptide of claim 6, wherein the assembly domain comprises a polypeptide sequence at least 50%, at least 60%, at least 70%, at least 75%, at least 80%, at least 85%, at least 90%, at least 95%, at least 96%, at least 97%, at least 98%, at least 99%, or 100% identical to any one of:
(SEQ ID NO: 25)
XXXXXXXMKMEELFKKHKIVAVLRANSVEEAIEKAVAVFAGGVHLIEIT
FTVPDADTVIKALSVLKEKGAIIGAGTVTSVEQCRKAVESGAEFIVSPH
LDEEISQFCKEKGVFYMPGVMTPTELVKAMKLGHDILKLFPGEVVGPQF
VKAMKGPFPNVKFVPTGGVNLDNVCKWFKAGVLAVGVGKALVKGKPDEV
REKAKKFVKKIRGCTEXXXXXXXXXXXXXXXX,
and/or
wherein the assembly domain comprises a polypeptide sequence at least 50%, at least 60%, at least 70%, at least 75%, at least 80%, at least 85%, at least 90%, at least 95%, at least 96%, at least 97%, at least 98%, at least 99%, or 100% identical to a first polypeptide sequence in Table 2 or Table 4.

9. (canceled)

10. The polypeptide claim 1, wherein the polypeptide comprises one or more amino acid residues at interface positions such that the polypeptide is capable of self-assemble to form a trimeric component of one-component nanostructure or a two-component nanostructure.

11. (canceled)

12. The polypeptide of claim 1, wherein the polypeptide self-assembles to form a trimeric component of a nanostructure, the C terminus of the assembly domain is accessible on the surface of the nanostructure.

13. The polypeptide of claim 1, wherein the polypeptide self-assembles to form a trimeric component, optionally wherein the distance from the C terminus of the assembly domain to the three-fold axis is less than 30 Å, less than 25 Å, or less than 20 Å, or between 10 Å and 30 Å, between 15 Å and 30 Å, between 15 Å and 25 Å, or between 20 Å and 25 Å.

14. (canceled)

15. The polypeptide of claim 1, wherein the polypeptide self-assembles to form a soluble trimer, the C terminus of the assembly domain is accessible on the surface of the soluble trimer and/or is proximal to the three-fold axis of the soluble trimer, optionally wherein the distance from the C terminus to the three-fold axis is less than 30 Å, less than 25 Å, or less than 20 Å, or between 10 Å and 30 Å, between 15 Å and 30 Å, between 15 Å and 25 Å, or between 20 Å and 25 Å.

16. The polypeptide of claim 1, wherein the polypeptide is a fusion protein comprising, in N- to C-terminal order, the assembly domain, optionally a polypeptide linker, and a heterologous polypeptide.

17. (canceled)

18. The polypeptide of claim 16, wherein the heterologous polypeptide is an antigen, wherein the antigen is an ectodomain of a surface protein of a pathogenic organism, optionally a virus or bacterium, or an antigenic fragment thereof.

19. The polypeptide of claim 16, wherein the heterologous polypeptide is an antigen, wherein the antigen is an OspA or antigenic fragment thereof, preferably an OspA of Borrelia burgdorferi sensu lato.

20.-21. (canceled)

22. A protein nanostructure, comprising a first component comprising a first polypeptide, and optionally a second component comprises a second polypeptide, wherein the first polypeptide is a polypeptide according to claim 1.

23. (canceled)

24. The nanostructure of claim 22, wherein the nanostructure comprises the second component and wherein the second component comprises a second assembly domain comprising a polypeptide sequence at least 50%, at least 60%, at least 70%, at least 75%, at least 80%, at least 85%, at least 90%, at least 95%, at least 96%, at least 97%, at least 98%, at least 99%, or 100% identical to:
(SEQ ID NO: 26)
NQHSHKDHETVRIAVVRARWHAEIVDACVSAFEAAMRDIGGDRFAVDVF
DVPGAYEIPLHARTLAETGRYGAVLGTAFVVNGGIYRHEFVASAVINGM
MNVQLNTGVPVLSAVLTPHNYDKSKAHTLLFLALFAVKGMEAARACVEI
LAAREKIAA;
or
(SEQ ID NO: 27)
NQHSHKDYETVRIAVVRARWHAEIVDACVSAFEAAMADIGGDRFAVDVF
DVPGAYEIPLHARTLAETGRYGAVLGTAFVVNGGIYRHEFVASAVIDGM
MNVQLSTGVPVLSAVLTPHRYRDSDAHTLLFLALFAVKGMEAARACVEI
LAAREKIAA

25.-35. (canceled)

36. A polynucleotide encoding the polypeptide of claim 1.

37. (canceled)

38. A pharmaceutical composition, comprising the nanostructure of claim 22, and a pharmaceutically acceptable carrier.

39. (canceled)

40. A host cell suitable comprising the polynucleotide of claim 36.

41. (canceled)

42. A method of generating an immune response to an antigen or to a pathogenic organism in a subject in need thereof or immunizing a subject against infection by a pathogen, comprising administering to the subject the pharmaceutical composition of claim 38, optionally via intramuscular injection or inhalation.

43.-44. (canceled)
